SEQUENCE LISTING (68 sequences)

SEQ ID NO: 1 - protein, 4 aa, Artificial Sequence
  HDEL tag
HDEL

SEQ ID NO: 2 - DNA, 12 bp, Artificial Sequence
  HDEL tag coding sequence
cacgacgagctg

SEQ ID NO: 3 - protein, 4 aa, Artificial Sequence
  KDEL tag
KDEL

SEQ ID NO: 4 - protein, 4 aa, Artificial Sequence
  DDEL tag
DDEL

SEQ ID NO: 5 - protein, 17 aa, Artificial Sequence
  Leader/Signal sequence
MKLSTILFTACATLAAA

SEQ ID NO: 6 - DNA, 51 bp, Artificial Sequence
  Coding sequence for Lip2 Leader/Signal Sequence
atgaagctgtctactattctctttactgcctgcgctactctcgccgctgct

SEQ ID NO: 7 - protein, 6 aa, Artificial Sequence
  His6 tag
HHHHHH

SEQ ID NO: 8 - DNA, 18 bp, Artificial Sequence
  His6 tag coding sequence
caccaccaccaccaccac

SEQ ID NO: 9 - protein, 341 aa, Homo sapiens
  MISC_FEATURE: mature FGE protein
SQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 10 - DNA, 1023 bp, Homo sapiens
  misc_feature: coding sequence of mature FGE protein
tcccaggaagccggcaccggagctggtgctggttctctggctggatcgtgcggatgtggcactcctcagcgacctggagctcatggctcctctgccgctgcccaccgatactctcgagaggctaacgctcctggtcctgtccccggagagcgacagctcgcccattctaagatggtgcctatccccgctggagttttcaccatgggcactgacgatcctcagatcaagcaggacggagaggctcctgctcgacgagtgaccattgacgccttttacatggatgcttacgaggtttcgaacactgagttcgagaagtttgtcaactctaccggatacctgactgaggccgagaagttcggtgactcgttcgtgtttgagggaatgctctccgagcaggtcaagaccaacatccagcaggctgtggctgccgctccttggtggctgcccgttaagggagctaactggcgacaccctgagggacctgactccaccattctgcaccgacctgatcatcccgtcctccacgtgtcttggaacgacgccgttgcttactgtacctgggctggcaagcgactgcctactgaggctgagtgggagtactcctgccgaggcggtctgcataaccgactcttcccttggggcaacaagctccagcccaagggtcagcactacgccaacatctggcagggcgagtttcctgtgaccaacactggagaggacggattccagggcaccgctcctgttgatgcttttccccctaacggttacggactgtacaacattgtcggtaacgcttgggagtggacctctgactggtggactgttcaccattcggtcgaggagaccctcaaccccaagggccctccctctggcaaggatcgagtcaagaagggaggctcctacatgtgccaccgatcttactgttaccgataccgatgcgccgctcgatcccagaacacccccgactcgtccgcctctaacctgggcttccgatgtgccgctgaccgactgcctactatggac

SEQ ID NO: 11 - protein, 314 aa, Streptomyces coelicolor
  MISC_FEATURE: FGE mature protein
MAVAAPSPAAAAEPGPAARPRSTRGQVRLPGGEFAMGDAFGEGYPADGETPVHTVRLRPFHIDETAVTNARFAAFVKATGHVTDAERFGSSAVFHLVVAAPDADVLGSAAGAPWWINVRGAHWRRPEGARSDITGRPNHPVVHVSWNDATAYARWAGKRLPTEAEWEYAARGGLAGRRYAWGDELTPGGRWRCNIWQGRFPHVNTAEDGHLSTAPVKSYRPNGHGLWNTAGNVWEWCSDWFSPTYYAESPTVDPHGPGTGAARVLRGGSYLCHDSYCNRYRVAARSSNTPDSSSGNLGFRCANDADLTSGSAAE

SEQ ID NO: 12 - DNA, 942 bp, Streptomyces coelicolor
  misc_feature: FGE protein coding sequence
atggctgttgctgctccctcgcctgctgctgctgccgagcccggtcctgctgctcgaccccgatctacccgaggacaggtgcgactgcctggcggtgagttcgctatgggcgacgcttttggagagggataccctgccgatggagagacccctgtgcacactgttcgactccgacccttccatatcgacgagaccgctgttactaacgcccgattcgccgcttttgtcaaggctaccggacacgtgactgatgccgagcgattcggctcctctgctgtttttcatctggtcgtggccgctcccgacgctgatgtcctgggctccgctgctggagctccttggtggatcaacgttcgaggtgcccactggcgacgacctgagggagctcgatctgacattaccggtcgacccaaccaccctgttgtccatgtctcctggaacgatgctaccgcttacgctcgatgggctggaaagcgactgcctactgaggctgagtgggagtacgctgctcgaggcggcctggctggtcgacgatacgcttggggagacgagctcacccccggtggacgatggcgatgcaacatttggcagggacgattccctcacgtcaacaccgccgaggacggccatctgtccactgctcccgtgaagtcttaccgacctaacggtcacggactctggaacaccgccggtaacgtctgggagtggtgttctgactggttttcgcccacctactacgccgagtctcctactgtcgacccccacggacctggtactggagctgctcgagttctgcgaggcggttcgtacctctgccatgactcctactgtaaccgataccgagtggccgctcgatcgtccaacacccccgactcttcgtccggcaacctcggtttccgatgcgccaacgatgctgacctgacttctggatctgccgctgag

SEQ ID NO: 13 - protein, 402 aa, Hemicentrotus pulcherrimus
  MISC_FEATURE: FGE mature protein
ENEDINQNISPTQSHTTATTEEELAEARGEEIDSDPTSEGSGAGEGCGCGSSALNRNHDEDALGLALEENLHDHVQEGAALKYSREANDPISMDHPEANVGAFPRTNQMNFIEGGTFRMGTDKAKIYLDGESPSRLVTLDPYYFDVYEVSNSEFELFVNTTSYITEAEKFGDSFVLEARISEEVKKDISQVVAAAPWWLPVKGAEWRHPEGPDSSISSRMDHPVTHISWNDATAYCQWAGKRLPTEAEWENAARGGLNNRLFPWGNKLMPKDHHRVNIWQGEFPKVNTAEDGYEGTCPVTAFEPNGYGLYNTVGNAWEWVADWWTTVHSPESQNNPVGPDEGTDKVKKGGSYMCHISYCYRYRCEARSQNSPDSSACNLGFRCAATNLPEDIPCSNCNDSTP

SEQ ID NO: 14 - DNA, 1206 bp, Hemicentrotus pulcherrimus
  misc_feature: FGE protein coding sequence
gagaacgaggacatcaaccagaacatttcgcctacccagtctcacaccactgccaccactgaggaagagctcgctgaggcccgaggcgaggagatcgactccgatcccacctctgagggctctggtgctggagagggatgcggttgtggctcctctgccctgaaccgaaaccacgacgaggatgctctgggtctcgccctggaggagaacctccacgaccatgttcaggaaggcgccgctctgaagtactcgcgagaggctaacgaccccatttctatggatcatcctgaggctaacgtcggtgccttcccccgaaccaaccagatgaacttcatcgagggcggtacctttcgaatgggaactgacaaggccaagatctacctggatggtgaatctccttcccgactggtgaccctggacccttactactttgatgtttacgaggtctctaactcggagttcgagctctttgttaacaccacttcttacatcaccgaggctgagaagttcggtgactcctttgtgctggaggcccgaatctctgaggaagtcaagaaggatatttctcaggtggtggctgctgctccttggtggctccccgtcaagggtgctgagtggcgacaccctgagggtcctgactcgtccatctcttcgcgaatggatcaccccgtgacccatatttcctggaacgacgctactgcctactgtcagtgggctggaaagcgactccctaccgaggctgagtgggagaacgctgctcgaggcggcctcaacaaccgactgttcccctggggcaacaagctgatgcctaaggaccaccatcgagttaacatttggcagggagagttccccaaggtcaacaccgctgaggacggatacgagggcacctgccccgtgactgcctttgagcctaacggctacggtctgtacaacactgtgggaaacgcttgggagtgggttgccgactggtggaccactgtccactcgcccgagtcccagaacaaccccgtcggtcctgacgagggaaccgataaggtcaagaagggcggctcctacatgtgccatatctcttactgttaccgataccgatgcgaggctcgatctcagaactcgcccgactcctctgcctgtaacctcggcttccgatgcgctgccaccaacctgcctgaggacattccttgttctaactgtaacgattccactccc

SEQ ID NO: 15 - protein, 343 aa, Bos Taurus
  MISC_FEATURE: FGE coding sequence mature sequence
AGGEEAGPEAGAPSLVGSCGCGNPQRPGAQGSSAAAHRYSREANAPGSVPGGRPSPPTKMVPIPAGVFTMGTDDPQIKQDGEAPARRVAIDAFYMDAYEVSNAEFEKFVNSTGYLTEAEKFGDSFVFEGMLSEQVKSDIQQAVAAAPWWLPVKGANWRHPEGPDSTVLHRPDHPVLHVSWNDAVAYCTWAGKRLPTEAEWEYSCRGGLQNRLFPWGNKLQPKGQHYANIWQGEFPVTNTGEDGFRGTAPVDAFPPNGYGLYNIVGNAWEWTSDWWTVHHSAEETINPKGPPSGKDRVKKGGSYMCHKSYCYRYRCAARSQNTPDSSASNLGFRCAADHLPTTG

SEQ ID NO: 16 - DNA, 1029 bp, Bos Taurus
  misc_feature: FGE protein coding sequence
gccggcggcgaggaagccggacctgaggccggcgctccctctctggttggatcgtgtggatgtggaaacccccagcgacctggcgctcagggttcctctgccgctgcccaccgatactctcgagaggctaacgctcctggctctgtccctggaggccgaccctcgccccctaccaagatggttcccatccctgccggcgtcttcaccatgggtactgacgatcctcagatcaagcaggacggagaggctcctgctcgacgagtggctattgacgctttttacatggatgcctacgaggtctctaacgctgagttcgagaagtttgtgaactcgaccggatacctgactgaggccgagaagttcggagactccttcgtttttgagggcatgctctccgagcaggtgaagtctgatattcagcaggctgttgctgccgctccttggtggctgcctgtcaagggagctaactggcgacatcccgagggtcctgactccaccgtgctgcaccgacccgatcatcctgtcctccacgtgtcttggaacgacgccgtcgcttactgtacctgggctggcaagcgactgcctactgaggctgagtgggagtactcttgccgaggtggactgcagaaccgactcttcccttggggtaacaagctccagcccaagggacagcactacgccaacatctggcagggagagtttcctgtgaccaacactggtgaagacggcttccgaggcaccgctcctgttgatgcttttccccctaacggttacggactctacaacatcgttggcaacgcctgggagtggacctccgactggtggactgtccaccattctgctgaggagactattaaccccaagggtcccccttctggaaaggatcgagtgaagaagggcggttcgtacatgtgccacaagtcctactgttaccgataccgatgcgccgctcgatcgcagaacacccccgactcgtccgcctccaacctgggattccgatgtgccgctgaccacctgcctactactgga

SEQ ID NO: 17 - protein, 299 aa, Mycobacterium tuberculosis
  MISC_FEATURE: FGE mature sequence
MLTELVDLPGGSFRMGSTRFYPEEAPIHTVTVRAFAVERHPVTNAQFAEFVSATGYVTVAEQPLDPGLYPGVDAADLCPGAMVFCPTAGPVDLRDWRQWWDWVPGACWRHPFGRDSDIADRAGHPVVQVAYPDAVAYARWAGRRLPTEAEWEYAARGGTTATYAWGDQEKPGGMLMANTWQGRFPYRNDGALGWVGTSPVGRFPANGFGLLDMIGNVWEWTTTEFYPHHRIDPPSTACCAPVKLATAADPTISQTLKGGSHLCAPEYCHRYRPAARSPQSQDTATTHIGFRCVADPVSG

SEQ ID NO: 18 - DNA, 897 bp, Mycobacterium tuberculosis
  misc_feature: FGE protein coding sequence
atgctgactgagctggttgacctccctggtggttccttccgaatgggatctacccgattttaccccgaggaggcccctatccacactgttaccgtccgagccttcgctgtcgagcgacatcccgtgaccaacgctcagttcgccgagtttgtttcggctactggctacgtgaccgttgctgagcagcctctggaccctggactctaccctggagtcgacgctgctgatctgtgccctggcgctatggtcttctgtcctaccgctggtcctgtggacctccgagattggcgacagtggtgggactgggtccctggtgcttgctggcgacacccttttggacgagactccgatattgctgaccgagctggacatcctgtcgtgcaggtggcttaccctgatgccgttgcttacgctcgatgggctggtcgacgactgcctactgaggctgagtgggagtacgctgctcgaggaggtaccactgctacctacgcttggggtgaccaggagaagcctggaggcatgctgatggctaacacctggcagggacgattcccttaccgaaacgatggagccctcggctgggttggtacctcccctgtcggacgattccctgctaacggctttggtctgctcgacatgatcggcaacgtgtgggagtggaccactaccgagttttacccccaccatcgaattgaccccccttctactgcttgctgtgctcctgttaagctcgctaccgctgctgatcctactatctcgcagaccctgaagggtggctcccacctctgcgctcccgagtactgtcatcgataccgacccgccgctcgatcccctcagtctcaggacaccgccactacccacattggttttcgatgtgttgctgaccctgtttcgggc

SEQ ID NO: 19 - protein, 525 aa, Artificial Sequence
  human iduronate sulfatase mature sequence
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 20 - DNA, 1575 bp, Artificial Sequence
  human iduronate sulfatase protein coding sequence
tctgagacccaggctaactcgactactgacgctctgaacgtgctcctgattattgttgacgacctgcgaccctccctcggttgctacggtgacaagctggtgcgatctcccaacatcgaccagctcgcttctcactcgctgctcttccagaacgcctttgctcagcaggccgtctgcgctccttcgcgagtgtccttcctgaccggacgacgacccgacaccactcgactctacgattttaactcctactggcgagtccacgccggtaacttctctaccatccctcagtactttaaggagaacggatacgtgactatgtccgtgggcaaggttttccaccccggtatttcctctaaccataccgacgattctccttactcctggtcttttcccccttaccacccctcgtccgagaagtacgagaacaccaagacttgccgaggccctgacggagagctgcatgctaacctgctctgtcccgtcgacgtgctggatgttcctgagggaaccctccccgataagcagtccactgagcaggccattcagctgctcgagaagatgaagacctcggcctcccccttctttctggctgtcggctaccacaagccccatatccctttccgataccctaaggagtttcagaagctgtaccccctcgagaacattaccctggctcccgaccctgaggttcctgatggtctgcctcccgtggcttacaacccttggatggacatccgacagcgagaggatgtgcaggccctgaacatctccgttccctacggtcccattcctgtcgacttccagcgaaagattcgacagtcttactttgcttctgtgtcgtacctggacacccaggttggtcgactgctctccgccctcgacgatctgcagctcgccaactcgaccatcattgctttcacttccgaccacggatgggccctgggagagcatggcgagtgggctaagtactctaacttcgacgttgccacccacgtccctctgatcttttacgttcctggacgaactgcctccctccctgaggctggtgaaaagctgttcccttacctcgacccctttgattccgcttctcagctgatggagcctggccgacagtctatggacctggtcgagctcgtgtcgctgttccccaccctggctggtctggctggcctgcaggtccctccccgatgccccgtgccttctttccacgttgagctctgtcgagagggaaagaacctgctcaagcatttccgatttcgagacctggaggaagacccctacctccctggcaacccccgagagctgatcgcctactcccagtacccccgaccttctgacattcctcagtggaactctgacaagccctcgctcaaggatatcaagattatgggctactccatccgaaccattgactaccgatacactgtttgggtcggtttcaaccccgacgagttcctggccaacttttcggatattcacgctggagagctgtacttcgtcgactctgatcccctccaggaccataacatgtacaacgactcgcagggcggtgacctcttccagctcctgatgcct

SEQ ID NO: 21 - protein, 487 aa, Artificial Sequence
  human PDI mature sequence
DAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAV

SEQ ID NO: 22 - DNA, 1461 bp, Artificial Sequence
  human PDI protein coding sequence
gacgcccccgaggaagaggaccacgtcctggtcctgcgaaagtctaacttcgccgaggccctggccgcccacaagtacctgctggtcgaattctacgccccctggtgcggccactgcaaggccctcgctcccgagtacgccaaggccgctggcaagctgaaggccgagggctctgagatccgactggccaaggtggacgccaccgaggaatctgacctggcccagcagtacggcgtgcgaggctaccccaccatcaagttcttccgaaacggcgacaccgcctctcccaaggagtacaccgccggacgagaggccgacgacatcgtgaactggctgaagaagcgaaccggacccgccgctactactctgcccgacggcgctgccgccgagtctctggtcgagtcctctgaggtggccgtgatcggcttcttcaaggacgtcgagtctgactctgccaagcagttcctgcaggccgccgaggccatcgacgacattcccttcggcatcacctctaactctgacgtgttctctaagtaccagctggacaaggacggcgtggtgctgttcaagaagttcgacgagggccgaaacaacttcgagggcgaggtgaccaaggaaaacctgctggacttcatcaagcacaaccagctgcccctggtgatcgagttcaccgagcagaccgcccccaagattttcggcggcgagatcaagacccacatcctgctgtttctgcccaagtctgtgtctgactacgacggcaagctgtctaacttcaagaccgccgctgagtctttcaagggcaagatcctgttcatcttcatcgactctgaccacaccgacaaccagcgaatcctcgagttcttcggcctgaagaaagaagaatgtcccgccgtccgactgatcaccctcgaggaagagatgaccaagtacaagcccgagtctgaggaactgaccgccgagcgaatcaccgagttctgccaccgattcctcgagggcaagatcaagccccacctgatgtctcaggaactgcccgaggactgggataagcagcccgtgaaggtgctggtgggcaagaacttcgaggacgtggccttcgacgagaagaagaacgttttcgtcgagttttacgctccttggtgtggacactgtaagcagctggcccccatctgggacaagctgggcgagacttacaaggaccacgagaacatcgtgatcgccaagatggactctaccgccaacgaggtcgaggccgtgaaggtccactcgttccccaccctgaagttctttcccgcctctgccgaccgaaccgtgatcgactacaacggcgagcgaaccctggacggcttcaagaagtttctcgagtctggcggccaggacggcgctggcgacgacgacgacctcgaggatctcgaagaagccgaggaacccgacatggaagaagacgacgaccagaaggccgtc

SEQ ID NO: 23 - protein, 33 aa, Artificial Sequence
  hFGE leader sequence
MAAPALGLVCGRCPELGLVLLLLLLSLLCGAAG

SEQ ID NO: 24 - protein, 480 aa, Homo sapiens
  MISC_FEATURE: sulfamidase protein mature sequence
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHN

SEQ ID NO: 25 - DNA, 1440 bp, Artificial Sequence
  coding sequence of mature sulfamidase (SGSH)
cgaccccgaaacgccctcctcctcctcgctgatgatggcggtttcgagtcgggtgcctacaacaactccgctatcgctacccctcacctcgacgctctggctcgacgatctctgctcttccgaaacgcctttacctccgtgtcctcttgctctccctcgcgagcttctctgctcactggactccctcagcaccagaacggaatgtacggcctgcatcaggacgttcaccatttcaactcttttgataaggtccgatcgctccctctgctcctgtcccaggctggtgttcgaaccggtatcattggaaagaagcacgtcggacccgagaccgtgtaccctttcgactttgcttacactgaggagaacggctccgttctgcaggtcggccgaaacatcacccgaattaagctcctggtccgaaagttcctccagactcaggacgatcgacccttctttctgtacgtggcctttcacgaccctcaccgatgcggacactctcagcctcagtacggtaccttctgtgagaagtttggaaacggcgagtccggtatgggacgaatccccgactggacccctcaggcttacgaccccctcgatgtcctggtgccttacttcgttcccaacacccctgctgctcgagctgacctcgctgctcagtacaccactgtcggccgaatggatcagggcgtgggtctcgttctgcaggagctgcgagacgctggtgtgctcaacgataccctggttatcttcacttctgacaacggtattccctttccttcgggacgaaccaacctgtactggcccggaactgctgagcctctcctggtctcgtcccctgagcaccctaagcgatggggacaggtttcggaggcttacgtctccctcctggacctcacccccactatcctggattggttctctattccctacccttcgtacgccatctttggatctaagaccattcatctgactggacgatccctcctgcctgctctcgaggctgagcctctgtgggctaccgtgttcggctcccagtctcaccatgaggttactatgtcctaccccatgcgatctgtccagcaccgacatttccgactcgtgcacaacctgaacttcaagatgccctttcctatcgaccaggatttctacgtctctcccacctttcaggacctcctgaaccgaaccactgccggccagcctaccggttggtacaaggatctccgacactactactaccgagctcgatgggagctgtacgaccgatcccgagatccccatgagacccagaacctggccactgaccctcgattcgctcagctcctggagatgctccgagaccagctggccaagtggcagtgggagacccacgatccctgggtgtgtgcccccgacggtgtgctcgaggagaagctgtccccccagtgtcagcccctgcataac

SEQ ID NO: 26 - protein, 163 aa, Artificial Sequence
  MNS1 anchorage domain (AA 1-163 of XP_502939.1)
MSFNIPKTTPNFSAKARKLEDQLWQASGLEKSKDSTLPLYKDKPYGEGFVARTTSGRRRRNIIYGVVVGLLFWAIYTFSRSLDGNVSLKDGIKDYEFKGWKGRGKPKTNWVAEQNAVKQAFVDSWNGYHKYAWGKDVYKPQTKTGKNMGPKPLGWFIVDSLDS

SEQ ID NO: 27 - DNA, 489 bp, Artificial Sequence
  Coding sequence for the MNS1 anchorage domain (AA 1-163 of XP_502939.1)
atgtcgttcaacattcccaagaccacccccaacttctcggctaaggctcgaaagctggaggatcagctctggcaggcttctggactcgagaagtccaaggactctaccctgcctctctacaaggataagccctacggagagggcttcgtggctcgaaccacttccggccgacgacgacgaaacatcatctacggcgtcgtggttggtctgctcttctgggccatctacaccttttctcgatcgctggacggtaacgtctctctcaaggacggaattaaggattacgagttcaagggctggaagggtcgaggaaagcccaagactaactgggtggccgagcagaacgctgttaagcaggcctttgtcgactcctggaacggctaccataagtacgcctggggcaaggatgtgtacaagccccagaccaagactggaaagaacatgggccccaagcctctgggatggttcatcgtggactctctggattcc

SEQ ID NO: 28 - protein, 118 aa, Artificial Sequence
  WBP1 anchorage domain (AA 400-505 of XP_502492.1)
DHLPTTGFTMLNPYYRLTLEQTGTTNFSAIYSTTFKIPDQHGVFTFNLDYKRPGYTFIEEKTRATIRHTANDEWPRSWEITNSWVYLTSAVMVVIAWFLFVVFYLFVGKADKEAVHKQ

SEQ ID NO: 29 - DNA, 354 bp, Articicial Sequence
  misc_feature: Coding sequence for the WBP1 anchorage domain (AA 400-505 of XP_502492.1)
gatcacctccccaccactggcttcaccatgctgaacccctactaccgactgaccctcgagcagactggcaccactaacttctccgccatctactctaccacttttaagattcctgaccagcatggcgtgttcacctttaacctcgattacaagcgacccggttacaccttcatcgaggagaagacccgagccactattcgacacaccgctaacgacgagtggccccgatcctgggagatcaccaactcttgggtctacctgacttcggccgtgatggtcgtgattgcttggttcctcttcgtggtgttctacctgtttgtgggaaaggctgataaggaagctgttcataagcag

SEQ ID NO: 30 - protein, 373 aa, Artificial Sequence
  ERp44 mature protein
EITSLDTENIDEILNNADVALVNFYADWCRFSQMLHPIFEEASDVIKEEFPNENQVVFARVDCDQHSDIAQRYRISKYPTLKLFRNGMMMKREYRGQRSVKALADYIRQQKSDPIQEIRDLAEITTLDRSKRNIIGYFEQKDSDNYRVFERVANILHDDCAFLSAFGDVSKPERYSGDNIIYKPPGHSAPDMVYLGAMTNFDVTYNWIQDKCVPLVREITFENGEELTEEGLPFLILFHMKEDTESLEIFQNEVARQLISEKGTINFLHADCDKFRHPLLHIQKTPADCPVIAIDSFRHMYVFGDFKDVLIPGKLKQFVFDLHSGKLHREFHHGPDPTDTAPGEQAQDVASSPPESSFQKLAPSEYRYTLLRD

SEQ ID NO: 31 - DNA, 1119 bp, Artificial Sequence
  Coding sequence for the ERp44 mature protein
gagattacttccctggatactgagaacatcgacgagattctgaacaacgccgacgtggccctggtcaacttctacgccgactggtgccgattttcccagatgctccaccccatcttcgaggaggcttctgatgtgattaaggaggagttccctaacgagaaccaggtcgtgtttgcccgagttgactgtgatcagcattctgacatcgctcagcgataccgaatttcgaagtaccccaccctgaagctcttccgaaacggaatgatgatgaagcgagagtaccgaggccagcgatcggttaaggccctggctgactacatccgacagcagaagtccgaccccatccaggagattcgagatctggccgagattaccactctcgaccgatctaagcgaaacatcattggttacttcgagcagaaggactcggataactaccgagtgtttgagcgagttgctaacatcctgcacgacgattgcgccttcctctctgcttttggagacgtctcgaagcccgagcgatactccggcgacaacatcatctacaagccccctggacattctgcccctgacatggtttacctgggcgctatgaccaacttcgacgtcacttacaactggattcaggataagtgtgttcccctcgtccgagagattacctttgagaacggcgaggagctgactgaggagggtctccctttcctgatcctctttcacatgaaggaggataccgagtccctggagattttccagaacgaggtggcccgacagctgatctccgagaagggaactattaacttcctccacgctgactgcgataagtttcgacaccccctgctccatatccagaagacccccgccgactgtcctgtcatcgctattgattctttccgacacatgtacgtcttcggcgactttaaggatgtgctgattcccggcaagctgaagcagttcgtgtttgacctgcactccggaaagctccatcgagagttccaccatggccccgaccctaccgatactgcccctggagagcaggcccaggacgttgcttcctctccccctgagtcgtccttccagaagctggccccctccgagtaccgatacaccctcctgcgagac

SEQ ID NO: 32 - protein, 378 aa, Artificial Sequence
  Fusion construct LIP2-BtFGE-6xHis-HDEL
MKLSTILFTACATLAAAAGGEEAGPEAGAPSLVGSCGCGNPQRPGAQGSSAAAHRYSREANAPGSVPGGRPSPPTKMVPIPAGVFTMGTDDPQIKQDGEAPARRVAIDAFYMDAYEVSNAEFEKFVNSTGYLTEAEKFGDSFVFEGMLSEQVKSDIQQAVAAAPWWLPVKGANWRHPEGPDSTVLHRPDHPVLHVSWNDAVAYCTWAGKRLPTEAEWEYSCRGGLQNRLFPWGNKLQPKGQHYANIWQGEFPVTNTGEDGFRGTAPVDAFPPNGYGLYNIVGNAWEWTSDWWTVHHSAEETINPKGPPSGKDRVKKGGSYMCHKSYCYRYRCAARSQNTPDSSASNLGFRCAADHLPTTGADHLPTTGHHHHHHHDEL

SEQ ID NO: 33 - protein, 4 aa, Artificial Sequence
  RDEL tag
RDEL

SEQ ID NO: 34 - protein, 12 aa, Artificial Sequence
  Conserved sequence of Iduronate Sulfatase
CAPSRVSFLTGR

SEQ ID NO: 35 - protein, 453 aa, Artificial Sequence
  MNS1-HpFGE- 6xHis fusion construct
MSFNIPKTTPNFSAKARKLEDQLWQASGLEKSKDSTLPLYKDKPYGEGFVARTTSGRRRRNIIYGVVVGLLFWAIYTFSRSLDGNVSLKDGIKDYEFKGWKGRGKPKTNWVAEQNAVKQAFVDSWNGYHKYAWGKDVYKPQTKTGKNMGPKPLGWFIVDSLDSMGTDKAKIYLDGESPSRLVTLDPYYFDVYEVSNSEFELFVNTTSYITEAEKFGDSFVLEARISEEVKKDISQVVAAAPWWLPVKGAEWRHPEGPDSSISSRMDHPVTHISWNDATAYCQWAGKRLPTEAEWENAARGGLNNRLFPWGNKLMPKDHHRVNIWQGEFPKVNTAEDGYEGTCPVTAFEPNGYGLYNTVGNAWEWVADWWTTVHSPESQNNPVGPDEGTDKVKKGGSYMCHISYCYRYRCEARSQNSPDSSACNLGFRCAATNLPEDIPCSNCNDSTPHHHHHH

SEQ ID NO: 36 - DNA, 1362 bp, Artificial Sequence
  Coding sequence for MNS1-HpFGE- 6xHis fusion construct
atgtcgttcaacattcccaagactacccctaacttctcggctaaggctcgaaagctggaggatcagctctggcaggcttctggactggagaagtccaaggactctaccctgcccctctacaaggataagccttacggagagggattcgtggctcgaaccacctccggccgacgacgacgaaacatcatctacggcgtcgtggttggtctgctcttctgggctatctacaccttttcccgatctctggacggcaacgtctccctcaaggacggtattaaggattacgagttcaagggatggaagggccgaggcaagcccaagaccaactgggtggctgagcagaacgccgtgaagcaggcttttgttgactcttggaacggataccacaagtacgcctggggcaaggatgtctacaagccccagaccaagactggaaagaacatgggccccaagcctctgggctggttcatcgtggactcgctcgattccatgggcaccgacaaggccaagatctacctggatggtgagtcgccctcccgactggttactctcgacccttactactttgatgtttacgaggtctctaactcggagttcgagctgtttgtcaacaccacttcttacatcaccgaggccgagaagttcggtgactcctttgtcctcgaggctcgaatctctgaggaagtcaagaaggatatttctcaggtggtggccgctgccccctggtggctccctgttaagggtgctgagtggcgacaccctgagggacctgactcctctatctcgtcccgaatggatcaccccgttacccatatttcctggaacgacgctactgcctactgtcagtgggctggcaagcgactgcctaccgaggctgagtgggagaacgctgctcgaggcggtctgaacaaccgactcttcccctggggaaacaagctcatgcctaaggaccaccatcgagtgaacatctggcagggcgagttccccaaggttaacaccgccgaggacggttacgagggaacctgccccgtgactgcttttgagcctaacggatacggcctgtacaacactgtcggaaacgcctgggagtgggtggctgactggtggaccactgttcactctcccgagtcgcagaacaaccccgttggtcctgacgagggaaccgataaggtcaagaagggaggctcgtacatgtgccatatttcttactgttaccgataccgatgcgaggcccgatcccagaactctcccgactcttcggcttgtaacctgggtttccgatgcgctgccaccaacctccctgaggacattccctgctctaactgtaacgactccactccccaccaccatcaccatcactaa

SEQ ID NO: 37 - protein, 519 aa, Artificial Sequence
  MNS1-BtFGE-6xHis fusion construct
MSFNIPKTTPNFSAKARKLEDQLWQASGLEKSKDSTLPLYKDKPYGEGFVARTTSGRRRRNIIYGVVVGLLFWAIYTFSRSLDGNVSLKDGIKDYEFKGWKGRGKPKTNWVAEQNAVKQAFVDSWNGYHKYAWGKDVYKPQTKTGKNMGPKPLGWFIVDSLDSGGEEAGPEAGAPSLVGSCGCGNPQRPGAQGSSAAAHRYSREANAPGSVPGGRPSPPTKMVPIPAGVFTMGTDDPQIKQDGEAPARRVAIDAFYMDAYEVSNAEFEKFVNSTGYLTEAEKFGDSFVFEGMLSEQVKSDIQQAVAAAPWWLPVKGANWRHPEGPDSTVLHRPDHPVLHVSWNDAVAYCTWAGKRLPTEAEWEYSCRGGLQNRLFPWGNKLQPKGQHYANIWQGEFPVTNTGEDGFRGTAPVDAFPPNGYGLYNIVGNAWEWTSDWWTVHHSAEETINPKGPPSGKDRVKKGGSYMCHKSYCYRYRCAARSQNTPDSSASNLGFRCAADHLPTTGADHLPTTGHHHHHH

SEQ ID NO: 38 - DNA, 1560 bp, Artificial Sequence
  Coding sequence for the MNS1-BtFGE-6xHis fusion construct
atgtcgttcaacattcccaagaccacccccaacttctcggctaaggctcgaaagctggaggatcagctctggcaggcttctggactcgagaagtccaaggactctaccctgcctctctacaaggataagccctacggagagggcttcgtggctcgaaccacttccggccgacgacgacgaaacatcatctacggcgtcgtggttggtctgctcttctgggccatctacaccttttctcgatcgctggacggtaacgtctctctcaaggacggaattaaggattacgagttcaagggctggaagggtcgaggaaagcccaagactaactgggtggccgagcagaacgctgttaagcaggcctttgtcgactcctggaacggctaccataagtacgcctggggcaaggatgtgtacaagccccagaccaagactggaaagaacatgggccccaagcctctgggatggttcatcgtggactctctggattccggcggcgaggaagccggtcctgaggctggagctccttctctggttggctcgtgcggctgtggaaacccccagcgacctggtgctcagggctcctctgccgctgcccaccgatactctcgagaggccaacgctcccggttctgtgcctggaggccgaccttcgccccctaccaagatggtgcccattcctgctggagttttcaccatgggcactgacgatcctcagatcaagcaggacggagaggctcctgctcgacgagttgccattgacgctttttacatggatgcttacgaggtttctaacgccgagttcgagaagtttgtcaactcgaccggatacctgactgaggccgagaagttcggagactccttcgtctttgagggcatgctctccgagcaggtcaagtctgacatccagcaggctgtggctgccgctccttggtggctgcccgttaagggtgctaactggcgacatcctgagggtcctgactccaccgtcctgcaccgacccgatcatcctgtcctccacgtgtcttggaacgacgccgtggcttactgtacctgggctggcaagcgactgcctactgaggctgagtgggagtactcttgccgaggtggactgcagaaccgactcttcccttggggtaacaagctccagcccaagggacagcactacgccaacatttggcagggcgagtttcctgtcaccaacactggcgaggacggtttccgaggaaccgctcccgtggatgcctttccccctaacggatacggcctgtacaacatcgtgggtaacgcttgggagtggacctccgactggtggactgttcaccattctgccgaggagaccattaaccctaagggccctccctctggcaaggaccgagtcaagaagggcggttcgtacatgtgccacaagtcctactgttaccgataccgatgcgccgctcgatcgcagaacacccctgactcttctgcttccaacctcggcttccgatgtgccgctgatcacctccccaccactggcgctgaccacctgcccactactggacaccaccaccaccaccattaa

SEQ ID NO: 39 - protein, 485 aa, Artificial Sequence
  Lip2pre-6xHis-BtFGE-WBP1 fusion construct
MKLSTILFTACATLAAAHHHHHHAGGEEAGPEAGAPSLVGSCGCGNPQRPGAQGSSAAAHRYSREANAPGSVPGGRPSPPTKMVPIPAGVFTMGTDDPQIKQDGEAPARRVAIDAFYMDAYEVSNAEFEKFVNSTGYLTEAEKFGDSFVFEGMLSEQVKSDIQQAVAAAPWWLPVKGANWRHPEGPDSTVLHRPDHPVLHVSWNDAVAYCTWAGKRLPTEAEWEYSCRGGLQNRLFPWGNKLQPKGQHYANIWQGEFPVTNTGEDGFRGTAPVDAFPPNGYGLYNIVGNAWEWTSDWWTVHHSAEETINPKGPPSGKDRVKKGGSYMCHKSYCYRYRCAARSQNTPDSSASNLGFRCAADHLPTTGADHLPTTGFTMLNPYYRLTLEQTGTTNFSAIYSTTFKIPDQHGVFTFNLDYKRPGYTFIEEKTRATIRHTANDEWPRSWEITNSWVYLTSAVMVVIAWFLFVVFYLFVGKADKEAVHKQ

SEQ ID NO: 40 - DNA, 1458 bp, Artificial Sequence
  Coding sequence for the Lip2pre-6xHis-BtFGE- WBP1 fusion construct
atgaagctgtctaccattctgtttactgcttgtgctaccctggctgctgcccaccaccatcaccatcacgctggcggagaagaggctggacccgaggctggagctccttccctggtgggatcgtgtggatgtggaaaccctcagcgacctggagctcagggttcttctgccgctgcccatcgatactcccgagaggctaacgctcctggttctgtgcctggcggacgaccttctcctcccaccaagatggtccccatccctgccggagttttcaccatgggtactgacgatcctcagatcaagcaggacggagaggctcctgctcgacgagttgccattgacgctttttacatggatgcctacgaggtctctaacgctgagttcgagaagtttgttaactccaccggatacctcactgaggccgagaagttcggcgactccttcgtctttgagggaatgctgtcggagcaggttaagtctgatattcagcaggctgtggctgccgctccttggtggctgcccgtcaagggagctaactggcgacatcccgagggtcctgactcgaccgttctgcaccgacccgatcatcctgttctccacgtgtcttggaacgacgctgtggcttactgcacctgggctggaaagcgactccccactgaggctgagtgggagtactcttgtcgaggtggcctgcagaaccgactcttcccttggggtaacaagctgcagcccaagggccagcactacgccaacatctggcagggagagtttcctgttaccaacactggagaggacggattccgaggtaccgctcctgtggatgcttttccccctaacggttacggcctctacaacatcgtgggcaacgcctgggagtggacctcggactggtggactgtccaccattctgctgaggagaccattaaccccaagggtcccccttctggcaaggatcgagtgaagaagggaggttcctacatgtgtcacaagtcgtactgctaccgataccgatgtgccgctcgatcccagaacacccctgactcgtctgcctcgaacctgggattccgatgcgccgctgaccatctgcctaccactggcgctgatcacctccccaccactggcttcaccatgctgaacccctactaccgactgaccctcgagcagactggcaccactaacttctctgccatctactccaccacttttaagattcctgaccagcatggtgtcttcacctttaacctcgattacaagcgacccggctacactttcatcgaggagaagacccgagccactattcgacacaccgctaacgacgagtggccccgatcttgggagatcaccaactcctgggtgtacctgacttcggccgtcatggtggtcattgcttggttcctgttcgtcgtgttttacctgttcgttggcaaggctgacaaggaagctgttcataagcagtaa

SEQ ID NO: 41 - protein, 380 aa, Artificial Sequence
  Chimeric Lip2pre-BtFGE-HpFGE-6xHis-HDEL fusion construct
MKLSTILFTACATLAAAAGGEEAGPEAGAPSLVGSCGCGNPQRPGAQGSSAAAHRYSREANAPGSVPGGRPSPPTKMVPIPAGVFTMGTDKAKIYLDGESPSRLVTLDPYYFDVYEVSNSEFELFVNTTSYITEAEKFGDSFVLEARISEEVKKDISQVVAAAPWWLPVKGAEWRHPEGPDSSISSRMDHPVTHISWNDATAYCQWAGKRLPTEAEWENAARGGLNNRLFPWGNKLMPKDHHRVNIWQGEFPKVNTAEDGYEGTCPVTAFEPNGYGLYNTVGNAWEWVADWWTTVHSPESQNNPVGPDEGTDKVKKGGSYMCHISYCYRYRCEARSQNSPDSSACNLGFRCAATNLPEDIPCSNCNDSTPHHHHHHHDEL

SEQ ID NO: 42 - DNA, 1143 bp, Artificial Sequence
  Coding sequence for Chimeric Lip2pre-BtFGE- HpFGE-6xHis-HDEL fusion construct
atgaagctgtctactattctgtttactgcttgcgctactctggctgccgctgccggaggcgaggaagctggtcccgaggctggtgctccctctctggtgggttcgtgcggctgtggaaacccccagcgacctggtgctcagggctcctctgccgctgcccaccgatactctcgagaggctaacgctcctggatcggtccctggcggtcgaccctctccccctaccaagatggtgcccatccctgccggtgttttcaccatgggaactgacaaggctaagatctacctggatggcgagtcgccttcccgactggtcaccctcgacccctactactttgatgtttacgaggtctctaactcggagttcgagctgtttgtgaacaccacttcttacatcactgaggccgagaagttcggtgactcctttgtcctcgaggctcgaatctctgaggaagtcaagaaggatatttctcaggtggtggctgccgctccttggtggctccccgttaagggtgctgagtggcgacaccctgagggtcctgactcgtccatctcttcgcgaatggatcaccctgtcacccatatttcctggaacgacgccactgcttactgtcagtgggctggcaagcgactgcccaccgaggctgagtgggagaacgctgctcgaggcggcctgaacaaccgactcttcccttggggaaacaagctcatgcccaaggaccaccatcgagtgaacatttggcagggcgagttccccaaggttaacaccgctgaggacggatacgagggtacctgccctgtgactgcttttgagcccaacggatacggcctctacaacactgtcggaaacgcctgggagtgggtggctgactggtggaccactgttcactcccccgagtctcagaacaaccccgttggacctgacgagggcaccgataaggtcaagaagggcggctcctacatgtgccatatctcttactgttaccgataccgatgcgaggcccgatcgcagaactcccctgactcctctgcttgtaacctgggtttccgatgcgccgctaccaacctccccgaggatattccctgttccaactgtaacgattccacccctcaccaccatcaccatcatcacgacgagctgtaa

SEQ ID NO: 43 - protein, 338 aa, Tupaia chinensis
  MISC_FEATURE: FGE
EEARTGAGATSAQGPCGCGTPQRPGSHGSSAAAHRYSREANVPGPVPGERQPEATKMVPIPAGVFTMGTDDPQIKQDGEAPARRVAIDAFYMDAYEVSNAEFEKFVNSTGYLTEAEKFGDSFVFEGMLSEQVKTGIQQAVAAAPWWLPVKGANWRHPEGPDSTILHRADHPVLHVSWNDAVAYCTWAGKRLPTEAEWEYSCRGGLQNRLFPWGNKLQPRGQHYANIWQGEFPVTNTAEDGFQGTAPVDAFPPNGYGLYNIVGNAWEWTSDWWTVYHSVEETLNPKGPPSGKDRVKKGGSYMCHKSYCYRYRCAARSQNTPDSSASNLGFRCAADRLPT

SEQ ID NO: 44 - DNA, 1014 bp, Artificial Sequence
  Coding sequence for Tupaia chinensis FGE
gaggaagcccgaactggtgctggtgctacttctgctcagggaccctgcggttgcggtactcctcagcgacccggttctcacggctcgtctgccgctgcccaccgatactctcgagaggctaacgttcctggacctgtccccggagagcgacagcctgaggccaccaagatggtccctatccccgctggcgtgttcaccatgggtactgacgatcctcagatcaagcaggacggtgaagctcctgctcgacgagttgccattgacgctttttacatggatgcctacgaggtgtccaacgctgagttcgagaagtttgttaactctaccggatacctgactgaggccgagaagttcggagactccttcgtctttgagggcatgctctctgagcaggttaagaccggcatccagcaggctgtggctgccgctccttggtggctgcctgtgaagggagctaactggcgacatcctgagggtcccgactccactattctgcaccgagctgatcatcctgtcctccacgtgtcttggaacgacgccgtcgcttactgtacctgggctggcaagcgactgcctactgaggctgagtgggagtactcctgccgaggcggtctgcagaaccgactcttcccttggggtaacaagctccagccccgaggacagcactacgccaacatctggcagggagagtttcctgtcaccaacactgctgaggacggattccagggcaccgctcctgtggatgcttttccccctaacggttacggactgtacaacattgttggaaacgcctgggagtggacctcggactggtggactgtgtaccattccgttgaggagaccctcaaccccaagggtcccccttctggaaaggatcgagtgaagaagggaggctcgtacatgtgccacaagtcctactgttaccgataccgatgcgccgctcgatctcagaacacccccgactcctctgcctcgaacctcggattccgatgtgctgctgaccgactgcccact

SEQ ID NO: 45 - protein, 341 aa, Monodelphis domestica
  MISC_FEATURE: FGE
AARGLGSEAGSAAADAAHPAGTCGCGSPQRPGTAAHRYSREANVAEPASAERPVLTSQMAHIPAGVFTMGTDEPQIKQDGEGPARRVRINSFYMDLYEVSNAEFERFVNSTGYVTEAEKFGDSFVFDSMLSDQVKSDIHQAVAAAPWWLPVKGANWRHPEGPDSSILHRRDHPVLHVSWNDAVAYCTWAGKRLPTEAEWEYSCRGGLENRLFPWGNKLQPKGQHYANIWQGEFPVSNTGEDGYQGTAPVTAFPPNGYGLYNIVGNAWEWTSDWWTVHHSADETLDPKGPPSGSDRVKKGGSYMCHKSYCYRYRCAARSQNTPDSSASNLGFRCAADRLPDT

SEQ ID NO: 46 - DNA, 1023 bp, Artificial Sequence
  Coding sequence for Monodelphis domestica FGE
gccgcccgaggtctgggttccgaggccggttccgccgccgccgacgccgctcaccctgctggcacttgtggttgtggttcccctcagcgacccggcaccgccgctcaccgatactctcgagaggctaacgtggctgagcctgcttctgccgagcgacctgtgctgacttcgcagatggctcacatccccgccggtgtcttcaccatgggaactgacgagccccagatcaagcaggatggagagggacctgcccgacgagttcgaattaactcgttttacatggacctctacgaggtctccaacgctgagttcgagcgatttgttaactccaccggttacgtcactgaggccgagaagttcggagactctttcgtttttgattccatgctgtctgaccaggtgaagtccgatatccatcaggctgtggccgctgccccctggtggctccctgtcaagggagctaactggcgacaccctgagggacctgactcctctattctgcaccgacgagatcatcccgtcctccacgtgtcttggaacgacgctgtggcctactgtacctgggctggaaagcgactgcctactgaggctgagtgggagtactcctgccgaggcggtctggagaaccgactctttccctggggcaacaagctccagcctaagggtcagcactacgctaacatctggcagggcgagttccccgtctccaacaccggagaggacggctaccagggcaccgctcctgtgactgcctttccccctaacggctacggtctgtacaacattgtgggtaacgcttgggagtggacctccgactggtggactgttcaccattctgccgacgagaccctcgatcccaagggacccccttctggctcggatcgagttaagaagggaggctcgtacatgtgccacaagtcctactgttaccgataccgatgcgctgcccgatctcagaacacccctgactcttccgcctctaacctgggcttccgatgtgctgctgaccgactgcctgacact

SEQ ID NO: 47 - protein, 329 aa, Gallus gallus
  MISC_FEATURE: FGE
GKETAPGGNCGCSASRSRGGEREAVATVRRYSAAANDGRSSGRGPMVAIPGGVFTMGTDEPEIQQDGEWPARRVHVNSFYMDQYEVSNQEFERFVNSTGYLTEAEKFGDSFVFEGMLSEEVKAEIHQAVAAAPWWLPVKGANWRQPEGPGSSILSRMDHPVLHVSWNDAVAFCTWAGKRLPTEAEWEYGCRGGLEKRLFPWGNKLQPKGQHYANIWQGVFPTNNTAEDGYKGTAPVTAFPPNGYGLYNIVGNAWEWTSDWWAVHHSADEAHNPKGPSSGTDRVKKGGSYMCHKSYCYRYRCAARSQNTPDSSASNLGFRCAADALPDPQ

SEQ ID NO: 48 - DNA, 987 bp, Artificial Sequence
  Coding sequence for Gallus gallus FGE
ggcaaggagactgcccctggcggtaactgcggttgttctgcttcccgatcccgaggtggagagcgagaggccgttgctactgtccgacgatactccgccgctgccaacgacggccgatcctctggccgaggtcccatggtggctatccctggcggtgttttcaccatgggaactgacgagcccgagattcagcaggatggcgagtggcctgctcgacgagtccacgtgaactcgttttacatggaccagtacgaggtttctaaccaggagttcgagcgatttgtcaactctaccggatacctgactgaggccgagaagttcggcgactctttcgtttttgagggaatgctctcggaggaagtcaaggccgagatccatcaggctgttgctgccgctccttggtggctgcctgtgaagggtgctaactggcgacagcctgagggacctggctcgtccattctgtcccgaatggaccaccccgttctccatgtctcttggaacgatgccgtcgctttctgtacctgggctggcaagcgactgcctactgaggctgagtgggagtacggatgccgaggcggcctggagaagcgactctttccctggggcaacaagctccagcctaagggtcagcactacgccaacatctggcagggcgtcttccccaccaacaacactgctgaggacggctacaagggcaccgcccctgtgactgcttttccccctaacggttacggactgtacaacattgtgggtaacgcctgggagtggacctctgactggtgggctgttcaccattctgccgatgaggctcacaaccccaagggaccttcttcgggcaccgaccgagtgaagaagggtggatcgtacatgtgccataagtcctactgttaccgataccgatgcgccgctcgatcccagaacacccccgattcctctgcctctaacctcggtttccgatgtgccgccgacgccctccccgaccctcag

SEQ ID NO: 49 - protein, 312 aa, Dendroctonus ponderosa
  MISC_FEATURE: FGE
ICDCGCSLNRDGQCNSEDNEINPSQKYKRDLNENPADNFDKSQMALIGKGIFEMGTNKPVFPSDFEGPARNVTIENSFYLDLYEVSNQQFYDFVRTTNYKTEAEQFGDSFVFEMSLPENQRNEHQDIRAAQAPWWIKLPDAYWKHPEGPKSTIEDRMNHPVAHVSWNDAVAYCEYVGKRLPTEAEWEMACRGGLRQKMYPWGNKLQPKGQHWANIWQGEFPKENTAEDGYIFTCPVDKFPPNQFGLYNMAGNVWEWVQDDWQTDPQNSRVKKGGSFLCHQSYCWRYRCAARSFNTKDSSAANLGFRCAADAR

SEQ ID NO: 50 - DNA, 936 bp, Artificial Sequence
  Coding sequence for the Dendroctonus ponderosa FGE
atttgcgactgcggctgctccctgaaccgagacggccagtgtaactccgaggacaacgagattaacccctcccagaagtacaagcgagacctgaacgagaaccccgccgacaacttcgataagtctcagatggctctcatcggcaagggaatttttgagatgggcaccaacaagcccgttttcccttcggactttgagggtcctgcccgaaacgtcactatcgagaactccttctacctggacctctacgaggtctctaaccagcagttctacgattttgtgcgaaccactaactacaagaccgaggctgagcagttcggtgactcgttcgtctttgagatgtccctgcccgagaaccagcgaaacgagcaccaggacatccgagctgctcaggctccttggtggattaagctccctgatgcttactggaagcatcccgagggacctaagtcgaccattgaggaccgaatgaaccaccccgtcgcccatgtgtcctggaacgatgccgtggcttactgtgagtacgttggcaagcgactgcctactgaggctgagtgggagatggcttgccgaggcggtctgcgacagaagatgtacccctggggaaacaagctccagcctaagggccagcactgggccaacatctggcagggagagttccccaaggagaacaccgctgaggacggatacatttttacttgtcctgtggataagttccctcccaaccagtttggcctctacaacatggccggtaacgtttgggagtgggtccaggacgattggcagaccgacccccagaactcccgagttaagaagggaggctctttcctgtgccatcagtcgtactgttggcgataccgatgcgccgctcgatctttcaacaccaaggactcctctgccgctaacctcggattccgatgtgctgctgacgcccga

SEQ ID NO: 51 - protein, 284 aa, Columba livia
  MISC_FEATURE: FGE
MVVIPGGVFTMGTDEPAIQQDGEWPVRKVHVNSFYMDRYEVSNEDFERFVNSTGYVTEAEKFGDSFVFEGMLSEEVKAEIHQAVAAAPWWLPVKGANWKHPEGPDSNISNRMDHPVLHVSWNDAVAFCTWAGKRLPTEAEWEYSCRGGLENRLFPWGNKLQPKGQHYANIWQGVFPTNNTAEDGYKGTAPVTAFPPNGYGLYNIVGNAWEWTADWWAVHHSTEEVHNPKGPSSGTDRVKKGGSYMCHKSYCYRYRCAARSQNTPDSSASNLGFRCAADASPELP

SEQ ID NO: 52 - DNA, 852 bp, Artificial Sequence
  Coding sequence for Columba livia FGE
atggtcgttattcccggaggagtttttactatgggtactgatgagcccgctatccagcaggacggagagtggcccgtgcgaaaggttcacgttaactctttctacatggaccgatacgaggtctcgaacgaggatttcgagcgatttgttaactccaccggctacgtcactgaggctgagaagtttggtgactcgttcgtctttgagggaatgctgtccgaggaagtcaaggctgagatccaccaggctgtggccgctgccccctggtggctccctgtgaagggagctaactggaagcatcccgagggccctgactctaacatttcgaaccgaatggatcaccccgtcctgcatgtgtcctggaacgatgctgttgccttctgtacctgggctggcaagcgactgcctactgaggccgagtgggagtactcttgccgaggcggtctggagaaccgactctttccctggggcaacaagctgcagcctaagggtcagcactacgctaacatctggcagggtgtgttccccaccaacaacactgccgaggacggctacaagggcaccgctcctgtgactgcctttccccctaacggttacggactctacaacattgttggaaacgcttgggagtggaccgctgactggtgggctgtgcaccattctactgaggaagtccacaaccccaagggaccttcctctggcaccgatcgagtcaagaagggaggctcctacatgtgccataagtcttactgttaccgataccgatgcgctgcccgatcccagaacacccccgactcgtccgcctctaacctgggattccgatgtgctgccgacgcttcgcctgagctgccc

SEQ ID NO: 53 - protein, 365 aa, Artificial Sequence
  Tupaia chinensis Lip2-TupFGE-His6-HDEL fusion construct
MKLSTILFTACATLAAAEEARTGAGATSAQGPCGCGTPQRPGSHGSSAAAHRYSREANVPGPVPGERQPEATKMVPIPAGVFTMGTDDPQIKQDGEAPARRVAIDAFYMDAYEVSNAEFEKFVNSTGYLTEAEKFGDSFVFEGMLSEQVKTGIQQAVAAAPWWLPVKGANWRHPEGPDSTILHRADHPVLHVSWNDAVAYCTWAGKRLPTEAEWEYSCRGGLQNRLFPWGNKLQPRGQHYANIWQGEFPVTNTAEDGFQGTAPVDAFPPNGYGLYNIVGNAWEWTSDWWTVYHSVEETLNPKGPPSGKDRVKKGGSYMCHKSYCYRYRCAARSQNTPDSSASNLGFRCAADRLPTHHHHHHHDEL

SEQ ID NO: 54 - DNA, 1098 bp, Artificial Sequence
  Coding sequence for the Lip2-TupFGE-His6-HDEL fusion protein
atgaagctttccaccatcctcttcacagcctgcgctaccctggctgccgccgaggaagcccgaactggtgctggtgctacttctgctcagggaccctgcggttgcggtactcctcagcgacccggttctcacggctcgtctgccgctgcccaccgatactctcgagaggctaacgttcctggacctgtccccggagagcgacagcctgaggccaccaagatggtccctatccccgctggcgtgttcaccatgggtactgacgatcctcagatcaagcaggacggtgaagctcctgctcgacgagttgccattgacgctttttacatggatgcctacgaggtgtccaacgctgagttcgagaagtttgttaactctaccggatacctgactgaggccgagaagttcggagactccttcgtctttgagggcatgctctctgagcaggttaagaccggcatccagcaggctgtggctgccgctccttggtggctgcctgtgaagggagctaactggcgacatcctgagggtcccgactccactattctgcaccgagctgatcatcctgtcctccacgtgtcttggaacgacgccgtcgcttactgtacctgggctggcaagcgactgcctactgaggctgagtgggagtactcctgccgaggcggtctgcagaaccgactcttcccttggggtaacaagctccagccccgaggacagcactacgccaacatctggcagggagagtttcctgtcaccaacactgctgaggacggattccagggcaccgctcctgtggatgcttttccccctaacggttacggactgtacaacattgttggaaacgcctgggagtggacctcggactggtggactgtgtaccattccgttgaggagaccctcaaccccaagggtcccccttctggaaaggatcgagtgaagaagggaggctcgtacatgtgccacaagtcctactgttaccgataccgatgcgccgctcgatctcagaacacccccgactcctctgcctcgaacctcggattccgatgtgctgctgaccgactgcccactcaccaccaccaccaccaccacgacgagctgtaa

SEQ ID NO: 55 - protein, 368 aa, Artificial Sequence
  Monodelphis domestica Lip2-MdFGE-His6-HDEL fusion construct
MKLSTILFTACATLAAAAARGLGSEAGSAAADAAHPAGTCGCGSPQRPGTAAHRYSREANVAEPASAERPVLTSQMAHIPAGVFTMGTDEPQIKQDGEGPARRVRINSFYMDLYEVSNAEFERFVNSTGYVTEAEKFGDSFVFDSMLSDQVKSDIHQAVAAAPWWLPVKGANWRHPEGPDSSILHRRDHPVLHVSWNDAVAYCTWAGKRLPTEAEWEYSCRGGLENRLFPWGNKLQPKGQHYANIWQGEFPVSNTGEDGYQGTAPVTAFPPNGYGLYNIVGNAWEWTSDWWTVHHSADETLDPKGPPSGSDRVKKGGSYMCHKSYCYRYRCAARSQNTPDSSASNLGFRCAADRLPDTHHHHHHHDEL

SEQ ID NO: 56 - DNA, 1107 bp, Artificial Sequence
  Coding sequence for the Lip2-MdFGE-His6-HDEL fusion protein
atgaagctttccaccatcctcttcacagcctgcgctaccctggctgccgccgccgcccgaggtctgggttccgaggccggttccgccgccgccgacgccgctcaccctgctggcacttgtggttgtggttcccctcagcgacccggcaccgccgctcaccgatactctcgagaggctaacgtggctgagcctgcttctgccgagcgacctgtgctgacttcgcagatggctcacatccccgccggtgtcttcaccatgggaactgacgagccccagatcaagcaggatggagagggacctgcccgacgagttcgaattaactcgttttacatggacctctacgaggtctccaacgctgagttcgagcgatttgttaactccaccggttacgtcactgaggccgagaagttcggagactctttcgtttttgattccatgctgtctgaccaggtgaagtccgatatccatcaggctgtggccgctgccccctggtggctccctgtcaagggagctaactggcgacaccctgagggacctgactcctctattctgcaccgacgagatcatcccgtcctccacgtgtcttggaacgacgctgtggcctactgtacctgggctggaaagcgactgcctactgaggctgagtgggagtactcctgccgaggcggtctggagaaccgactctttccctggggcaacaagctccagcctaagggtcagcactacgctaacatctggcagggcgagttccccgtctccaacaccggagaggacggctaccagggcaccgctcctgtgactgcctttccccctaacggctacggtctgtacaacattgtgggtaacgcttgggagtggacctccgactggtggactgttcaccattctgccgacgagaccctcgatcccaagggacccccttctggctcggatcgagttaagaagggaggctcgtacatgtgccacaagtcctactgttaccgataccgatgcgctgcccgatctcagaacacccctgactcttccgcctctaacctgggcttccgatgtgctgctgaccgactgcctgacactcatcaccatcatcaccaccacgacgagctgtaa

SEQ ID NO: 57 - protein, 356 aa, Artificial Sequence
  Gallus gallus Lip2-GgFGE-His6-HDEL fusion construct
MKLSTILFTACATLAAAGKETAPGGNCGCSASRSRGGEREAVATVRRYSAAANDGRSSGRGPMVAIPGGVFTMGTDEPEIQQDGEWPARRVHVNSFYMDQYEVSNQEFERFVNSTGYLTEAEKFGDSFVFEGMLSEEVKAEIHQAVAAAPWWLPVKGANWRQPEGPGSSILSRMDHPVLHVSWNDAVAFCTWAGKRLPTEAEWEYGCRGGLEKRLFPWGNKLQPKGQHYANIWQGVFPTNNTAEDGYKGTAPVTAFPPNGYGLYNIVGNAWEWTSDWWAVHHSADEAHNPKGPSSGTDRVKKGGSYMCHKSYCYRYRCAARSQNTPDSSASNLGFRCAADALPDPQHHHHHHHDEL

SEQ ID NO: 58 - DNA, 1071 bp, Artificial Sequence
  Coding sequence for the Lip2-GgFGE-His6-HDEL fusion protein
atgaagctttccaccatcctcttcacagcctgcgctaccctggctgccgccggcaaggagactgcccctggcggtaactgcggttgttctgcttcccgatcccgaggtggagagcgagaggccgttgctactgtccgacgatactccgccgctgccaacgacggccgatcctctggccgaggtcccatggtggctatccctggcggtgttttcaccatgggaactgacgagcccgagattcagcaggatggcgagtggcctgctcgacgagtccacgtgaactcgttttacatggaccagtacgaggtttctaaccaggagttcgagcgatttgtcaactctaccggatacctgactgaggccgagaagttcggcgactctttcgtttttgagggaatgctctcggaggaagtcaaggccgagatccatcaggctgttgctgccgctccttggtggctgcctgtgaagggtgctaactggcgacagcctgagggacctggctcgtccattctgtcccgaatggaccaccccgttctccatgtctcttggaacgatgccgtcgctttctgtacctgggctggcaagcgactgcctactgaggctgagtgggagtacggatgccgaggcggcctggagaagcgactctttccctggggcaacaagctccagcctaagggtcagcactacgccaacatctggcagggcgtcttccccaccaacaacactgctgaggacggctacaagggcaccgcccctgtgactgcttttccccctaacggttacggactgtacaacattgtgggtaacgcctgggagtggacctctgactggtgggctgttcaccattctgccgatgaggctcacaaccccaagggaccttcttcgggcaccgaccgagtgaagaagggtggatcgtacatgtgccataagtcctactgttaccgataccgatgcgccgctcgatcccagaacacccccgattcctctgcctctaacctcggtttccgatgtgccgccgacgccctccccgaccctcagcatcaccatcaccatcatcacgacgagctgtag

SEQ ID NO: 59 - protein, 339 aa, Artificial Sequence
  Dendroctonus ponderosa Lip2-DpFGE-His6-HDEL fusion construct
MKLSTILFTACATLAAAICDCGCSLNRDGQCNSEDNEINPSQKYKRDLNENPADNFDKSQMALIGKGIFEMGTNKPVFPSDFEGPARNVTIENSFYLDLYEVSNQQFYDFVRTTNYKTEAEQFGDSFVFEMSLPENQRNEHQDIRAAQAPWWIKLPDAYWKHPEGPKSTIEDRMNHPVAHVSWNDAVAYCEYVGKRLPTEAEWEMACRGGLRQKMYPWGNKLQPKGQHWANIWQGEFPKENTAEDGYIFTCPVDKFPPNQFGLYNMAGNVWEWVQDDWQTDPQNSRVKKGGSFLCHQSYCWRYRCAARSFNTKDSSAANLGFRCAADARHHHHHHHDEL

SEQ ID NO: 60 - DNA, 1020 bp, Artificial Sequence
  Coding sequence for the Lip2-DpFGE-His6-HDEL fusion protein
atgaagctttccaccatcctcttcacagcctgcgctaccctggctgccgccatttgcgactgcggctgctccctgaaccgagacggccagtgtaactccgaggacaacgagattaacccctcccagaagtacaagcgagacctgaacgagaaccccgccgacaacttcgataagtctcagatggctctcatcggcaagggaatttttgagatgggcaccaacaagcccgttttcccttcggactttgagggtcctgcccgaaacgtcactatcgagaactccttctacctggacctctacgaggtctctaaccagcagttctacgattttgtgcgaaccactaactacaagaccgaggctgagcagttcggtgactcgttcgtctttgagatgtccctgcccgagaaccagcgaaacgagcaccaggacatccgagctgctcaggctccttggtggattaagctccctgatgcttactggaagcatcccgagggacctaagtcgaccattgaggaccgaatgaaccaccccgtcgcccatgtgtcctggaacgatgccgtggcttactgtgagtacgttggcaagcgactgcctactgaggctgagtgggagatggcttgccgaggcggtctgcgacagaagatgtacccctggggaaacaagctccagcctaagggccagcactgggccaacatctggcagggagagttccccaaggagaacaccgctgaggacggatacatttttacttgtcctgtggataagttccctcccaaccagtttggcctctacaacatggccggtaacgtttgggagtgggtccaggacgattggcagaccgacccccagaactcccgagttaagaagggaggctctttcctgtgccatcagtcgtactgttggcgataccgatgcgccgctcgatctttcaacaccaaggactcctctgccgctaacctcggattccgatgtgctgctgacgcccgacaccaccaccaccaccaccacgacgagctgtag

SEQ ID NO: 61 - protein, 311 aa, Artificial Sequence
  Columba livia Lip2-ClFGE-His6-HDEL fusion construct
MKLSTILFTACATLAAAMVVIPGGVFTMGTDEPAIQQDGEWPVRKVHVNSFYMDRYEVSNEDFERFVNSTGYVTEAEKFGDSFVFEGMLSEEVKAEIHQAVAAAPWWLPVKGANWKHPEGPDSNISNRMDHPVLHVSWNDAVAFCTWAGKRLPTEAEWEYSCRGGLENRLFPWGNKLQPKGQHYANIWQGVFPTNNTAEDGYKGTAPVTAFPPNGYGLYNIVGNAWEWTADWWAVHHSTEEVHNPKGPSSGTDRVKKGGSYMCHKSYCYRYRCAARSQNTPDSSASNLGFRCAADASPELPHHHHHHHDEL

SEQ ID NO: 62 - DNA, 936 bp, Artificial Sequence
  Coding sequence for the Lip2-ClFGE-His6-HDEL fusion protein
atgaagctttccaccatcctcttcacagcctgcgctaccctggctgccgccatggtcgttattcccggaggagtttttactatgggtactgatgagcccgctatccagcaggacggagagtggcccgtgcgaaaggttcacgttaactctttctacatggaccgatacgaggtctcgaacgaggatttcgagcgatttgttaactccaccggctacgtcactgaggctgagaagtttggtgactcgttcgtctttgagggaatgctgtccgaggaagtcaaggctgagatccaccaggctgtggccgctgccccctggtggctccctgtgaagggagctaactggaagcatcccgagggccctgactctaacatttcgaaccgaatggatcaccccgtcctgcatgtgtcctggaacgatgctgttgccttctgtacctgggctggcaagcgactgcctactgaggccgagtgggagtactcttgccgaggcggtctggagaaccgactctttccctggggcaacaagctgcagcctaagggtcagcactacgctaacatctggcagggtgtgttccccaccaacaacactgccgaggacggctacaagggcaccgctcctgtgactgcctttccccctaacggttacggactctacaacattgttggaaacgcttgggagtggaccgctgactggtgggctgtgcaccattctactgaggaagtccacaaccccaagggaccttcctctggcaccgatcgagtcaagaagggaggctcctacatgtgccataagtcttactgttaccgataccgatgcgctgcccgatcccagaacacccccgactcgtccgcctctaacctgggattccgatgtgctgccgacgcttcgcctgagctgccccaccaccaccatcaccatcacgacgagctgtaa

SEQ ID NO: 63 - protein, 447 aa, Artificial Sequence
  MNS1-ClFGE fusion construct
MSFNIPKTTPNFSAKARKLEDQLWQASGLEKSKDSTLPLYKDKPYGEGFVARTTSGRRRRNIIYGVVVGLLFWAIYTFSRSLDGNVSLKDGIKDYEFKGWKGRGKPKTNWVAEQNAVKQAFVDSWNGYHKYAWGKDVYKPQTKTGKNMGPKPLGWFIVDSLDSMVVIPGGVFTMGTDEPAIQQDGEWPVRKVHVNSFYMDRYEVSNEDFERFVNSTGYVTEAEKFGDSFVFEGMLSEEVKAEIHQAVAAAPWWLPVKGANWKHPEGPDSNISNRMDHPVLHVSWNDAVAFCTWAGKRLPTEAEWEYSCRGGLENRLFPWGNKLQPKGQHYANIWQGVFPTNNTAEDGYKGTAPVTAFPPNGYGLYNIVGNAWEWTADWWAVHHSTEEVHNPKGPSSGTDRVKKGGSYMCHKSYCYRYRCAARSQNTPDSSASNLGFRCAADASPELP

SEQ ID NO: 64 - DNA, 1341 bp, Artificial Sequence
  Coding sequence for the MNS1-ClFGE fusion protein
atgtcgttcaacattcccaagaccacccccaacttctcggctaaggctcgaaagctggaggatcagctctggcaggcttctggactcgagaagtccaaggactctaccctgcctctctacaaggataagccctacggagagggcttcgtggctcgaaccacttccggccgacgacgacgaaacatcatctacggcgtcgtggttggtctgctcttctgggccatctacaccttttctcgatcgctggacggtaacgtctctctcaaggacggaattaaggattacgagttcaagggctggaagggtcgaggaaagcccaagactaactgggtggccgagcagaacgctgttaagcaggcctttgtcgactcctggaacggctaccataagtacgcctggggcaaggatgtgtacaagccccagaccaagactggaaagaacatgggccccaagcctctgggatggttcatcgtggactctctggattccatggtcgttattcccggaggagtttttactatgggtactgatgagcccgctatccagcaggacggagagtggcccgtgcgaaaggttcacgttaactctttctacatggaccgatacgaggtctcgaacgaggatttcgagcgatttgttaactccaccggctacgtcactgaggctgagaagtttggtgactcgttcgtctttgagggaatgctgtccgaggaagtcaaggctgagatccaccaggctgtggccgctgccccctggtggctccctgtgaagggagctaactggaagcatcccgagggccctgactctaacatttcgaaccgaatggatcaccccgtcctgcatgtgtcctggaacgatgctgttgccttctgtacctgggctggcaagcgactgcctactgaggccgagtgggagtactcttgccgaggcggtctggagaaccgactctttccctggggcaacaagctgcagcctaagggtcagcactacgctaacatctggcagggtgtgttccccaccaacaacactgccgaggacggctacaagggcaccgctcctgtgactgcctttccccctaacggttacggactctacaacattgttggaaacgcttgggagtggaccgctgactggtgggctgtgcaccattctactgaggaagtccacaaccccaagggaccttcctctggcaccgatcgagtcaagaagggaggctcctacatgtgccataagtcttactgttaccgataccgatgcgctgcccgatcccagaacacccccgactcgtccgcctctaacctgggattccgatgtgctgccgacgcttcgcctgagctgccc

SEQ ID NO: 65 - protein, 10 aa, Artificial Sequence
  c-myc protein tag
EQKLISEEDL

SEQ ID NO: 66 - DNA, 33 bp, Artificial Sequence
  Coding sequence for the c-myc protein tag
gaacaaaaactcatctcagaagaggatctgtaa

SEQ ID NO: 67 - protein, 457 aa, Artificial Sequence
  MNS1-ClFGE-c-myc fusion construct
MSFNIPKTTPNFSAKARKLEDQLWQASGLEKSKDSTLPLYKDKPYGEGFVARTTSGRRRRNIIYGVVVGLLFWAIYTFSRSLDGNVSLKDGIKDYEFKGWKGRGKPKTNWVAEQNAVKQAFVDSWNGYHKYAWGKDVYKPQTKTGKNMGPKPLGWFIVDSLDSMVVIPGGVFTMGTDEPAIQQDGEWPVRKVHVNSFYMDRYEVSNEDFERFVNSTGYVTEAEKFGDSFVFEGMLSEEVKAEIHQAVAAAPWWLPVKGANWKHPEGPDSNISNRMDHPVLHVSWNDAVAFCTWAGKRLPTEAEWEYSCRGGLENRLFPWGNKLQPKGQHYANIWQGVFPTNNTAEDGYKGTAPVTAFPPNGYGLYNIVGNAWEWTADWWAVHHSTEEVHNPKGPSSGTDRVKKGGSYMCHKSYCYRYRCAARSQNTPDSSASNLGFRCAADASPELPEQKLISEEDL

SEQ ID NO: 68 - DNA, 1374 bp, Artificial Sequence
  Coding sequence for the MNS1-ClFGE-c-myc fusion protein
atgtcgttcaacattcccaagaccacccccaacttctcggctaaggctcgaaagctggaggatcagctctggcaggcttctggactcgagaagtccaaggactctaccctgcctctctacaaggataagccctacggagagggcttcgtggctcgaaccacttccggccgacgacgacgaaacatcatctacggcgtcgtggttggtctgctcttctgggccatctacaccttttctcgatcgctggacggtaacgtctctctcaaggacggaattaaggattacgagttcaagggctggaagggtcgaggaaagcccaagactaactgggtggccgagcagaacgctgttaagcaggcctttgtcgactcctggaacggctaccataagtacgcctggggcaaggatgtgtacaagccccagaccaagactggaaagaacatgggccccaagcctctgggatggttcatcgtggactctctggattccatggtcgttattcccggaggagtttttactatgggtactgatgagcccgctatccagcaggacggagagtggcccgtgcgaaaggttcacgttaactctttctacatggaccgatacgaggtctcgaacgaggatttcgagcgatttgttaactccaccggctacgtcactgaggctgagaagtttggtgactcgttcgtctttgagggaatgctgtccgaggaagtcaaggctgagatccaccaggctgtggccgctgccccctggtggctccctgtgaagggagctaactggaagcatcccgagggccctgactctaacatttcgaaccgaatggatcaccccgtcctgcatgtgtcctggaacgatgctgttgccttctgtacctgggctggcaagcgactgcctactgaggccgagtgggagtactcttgccgaggcggtctggagaaccgactctttccctggggcaacaagctgcagcctaagggtcagcactacgctaacatctggcagggtgtgttccccaccaacaacactgccgaggacggctacaagggcaccgctcctgtgactgcctttccccctaacggttacggactctacaacattgttggaaacgcttgggagtggaccgctgactggtgggctgtgcaccattctactgaggaagtccacaaccccaagggaccttcctctggcaccgatcgagtcaagaagggaggctcctacatgtgccataagtcttactgttaccgataccgatgcgctgcccgatcccagaacacccccgactcgtccgcctctaacctgggattccgatgtgctgccgacgcttcgcctgagctgcccgaacaaaaactcatctcagaagaggatctgtaa

(19) United States
(12) Patent Application Publication
(10) Pub. No.: US 2022/0204952 A1
(43) Pub. Date: Jun. 30, 2022

(54) PRODUCTION OF CATALYTICALLY ACTIVE TYPE I SULFATASE

(71) Applicant: Oxyrane UK Limited, Manchester (GB)

(72) Inventors: Wouter Vervecken, Landskouter (BE); Stefan Simonne Prudent Eugène Christine Ryckaert, Sint-Amandsberg (BE); Albena Vergilieva Valevska, Astene (BE)

(21) Appl. No.: 17/574,406

(22) Filed: Jan. 12, 2022

Related U.S. Application Data
(63) Continuation of application No. 14/773,234, filed on Sep. 4, 2015, now abandoned.
(60) Provisional application No. 61/790,530, filed on Mar. 15, 2013.
(60) Provisional application No. 61/773,034, filed on Mar. 5, 2013.

(51) Int. Cl. C12N 9/16; G01N 33/50; C12N 15/81

(52) U.S. Cl. CPC C12N 9/16; C12Y 301/06; A61K 38/00; C12N 15/815; C12Y 301/06013; G01N 33/5017

(57) ABSTRACT

The present disclosure provides methods for producing activated type I sulfatases, or functional fragments thereof, using Formylglycine Generating Enzymes (FGEs). Also featured by the disclosure are recombinant fungal (e.g., Yarrowia lipolytica) cells expressing the FGE and, in some embodiments, type I sulfatases, or functional fragments thereof, and/or additional accessory enzymes. The disclosure also provides activated type I sulfatases or functional fragments thereof, made by the disclosed methods and therapeutic methods using the activated type I sulfatases or functional fragments thereof.

### CROSS-REFERENCE TO RELATED APPLICATIONS

This application is a continuation and claims priority to U.S. National Stage application Ser. No. 14/773,234, filed Sep. 4, 2015, which claims priority of International Application No. PCT/M2014/059464, filed Mar. 5, 2014, which claims priority of U.S. Provisional Nos. 61/790,530, filed Mar. 15, 2013, and U.S. Provisional Application No. 61/773,034, filed Mar. 5, 2013. The contents of all of the prior applications are incorporated herein by reference in their entirety.

### SEQUENCE LISTING

This application contains a Sequence Listing that has been submitted electronically as an ASCII text file named 18990-0037002.SEQ.txt. The ASCII text file, created on Jan. 10, 2022, is 139,264 bytes in size. The material in the ASCII text file is hereby incorporated by reference in its entirety.

### TECHNICAL FIELD

This document relates to methods and materials, including genetically engineered fungal cells, useful for the production of type I sulfatase enzymes or functional fragments thereof, in their catalytically active form.

### BACKGROUND

Sulfatases catalyze the hydrolysis of sulfate esters (e.g., sulfates) of substrates including steroids, complex cell surface carbohydrates and proteins. The absence of an active individual type I sulfatase has been implicated in a number of pathophysical conditions, namely lysosomal storage disorders which includes mucopolysaccharidoses (MPS), such as MPSII, MPSIIA, MPSIVA, MPSVI, and metachromatic leukodystrophy.

Thus, a method of making type I sulfatase with a high level of activity for use in such disorders would be extremely valuable.

### SUMMARY

This document provides methods and materials based on, inter alia, the discovery by the inventors that catalytically active type I sulfatases can be produced in recombinant fungi expressing type I sulfatase-activating enzymes (FGEs) from a variety of species.

The present document provides a first method for making a type I sulfatase, or a functional fragment of a type I sulfatase, in an active form. The method includes: (a) providing a fungal cell genetically engineered such that, when transformed with a polynucleotide encoding a type I sulfatase, or a functional fragment of a type I sulfatase, the cell has the ability to produce the type I sulfatase, or the functional fragment of the type I sulfatase in an active form, or an increased level of the type I sulfatase, or the functional fragment of the type I sulfatase in an active form; and (b) introducing into the cell a nucleic acid encoding the type I sulfatase, or a functional fragment of the type I sulfatase. The encoded type I sulfatase, or functional fragment of the type I sulfatase, without an activation step, is in an inactive form. After the introduction, the cell produces, or produces at an increased level, the type I sulfatase, or a functional fragment of the type I sulfatase, in an active form.

The document also features a second method for making a type I sulfatase, or a functional fragment of a type I sulfatase, in an active form. The method includes: (a) providing a fungal cell genetically engineered to produce a produce a protein with the type I sulfatase activating activity of a Formylglycine Generating Enzyme (FGE); and (b) introducing into the cell a nucleic acid encoding a type I sulfatase, or a functional fragment of the type I sulfatase. The encoded type I sulfatase, or the encoded functional fragment of the type I sulfatase, without an activation step, is in an inactive form. After the introduction, the cell produces, or produces at an increased level, the type I sulfatase, or the functional fragment of the type I sulfatase, in an active form.

In addition, the document provides a third method for making a type I sulfatase, or a functional fragment of a type I sulfatase, in an active form. The method includes: (a) providing a fungal cell genetically engineered to produce a type I sulfatase, or a functional fragment of the type I sulfatase, the encoded type I sulfatase, or the encoded functional fragment of the type I sulfatase, without an activation step, being in an inactive form; and (b) introducing into the cell a nucleic acid encoding a produce a protein with the type I sulfatase activating activity of a Formylglycine Generating Enzyme (FGE). After the introduction, the cell produces, or produces at an increased level, the type I sulfatase, or functional fragment of the type I sulfatase, in an active form.

In the second and third methods, the protein with the type I sulfatase activating activity of a FGE can include or be any of (a)-(f) as follows: (a) a mature wild type FGE polypeptide; (b) a functional fragment of a mature wild type FGE polypeptide comprising at least 50 (e.g., at least: 60; 70; 80; 90; 100; 125; 150; 175; 200; 225; 250; 275; 300; 325; 350; 400; 450; 500; or more) consecutive amino acids of the mature wild type FGE; (c) a polypeptide with at least 80% (e.g., at least: 85%; 88%; 90%; 92%; 95%; 98%; 99%; or 99.5%) identity to (a); (d) a polypeptide with at least 90% (e.g., at least: 92%; 95%; 98%; 99%; or 99.5%) identity to (b); (e) (a) but with no more than 10 (e.g., no more than 8; 7; 6; 5; 4; 3; 2; or 1) conservative substitution(s); or (f) (b) but with no more than 5 (e.g., no more than 4; 3; 2; or 1) conservative substitutions(s). In all of the methods in which an FGE is involved, the FGE can be the following mature wild type proteins and functional fragments of the mature wild type proteins as well as variants (listed above) of either: mature wild type protein SCO7548; mature wild type protein Rv0712; mature wild type sulfatase modifying factor 1; mature wild type C-alpha-formylglycine-generating enzyme; or mature wild type sulfatase-modifying factor 1. Also useful for the production methods of the disclosure are fusion proteins containing any of the mature wild type proteins, functional fragments, and variants of both. Moreover, the FGE can be a prokaryotic FGE (e.g., a FGE from Mycobacterium tuberculosis or Streptomyces coelicolor). Alternatively, the FGE can be a eukaryotic FGE (e.g., a FGE of Homo sapiens, Bos taurus, Hemicentrotus pulcherrimus, Tupaia chinensis, Monodelphis domestica, Gallus gallus, Dendroctonus ponderosa, or Columba livia).

In addition, in any of the active type I sulfatase production methods described in the present disclosure, any of the proteins with the type I sulfatase activating activity of a FGE, fusion proteins containing such proteins, can further include a ER targeting motif such as HDEL (SEQ ID NO: 1), KDEL (SEQ ID NO: 3), DDEL (SEQ ID NO: 4), RDEL (SEQ ID NO: 33), a yeast MNS1 transmembrane anchor polypeptide (such as the Yarrowia lipolytica MNS1 transmembrane anchor polypeptide), a yeast WBP1 transmembrane anchor polypeptide (such as the Yarrowia lipolytica WBP1 transmembrane anchor polypeptide), or the transmembrane parts of Secretory-12 (SEC12), Glucosidase-1 (GLS1), or STaurosporine Temperature Sensitive-3 (STT3). The ER targeting motif can be fused to the N-terminus or the C-terminus of any of the proteins with the type I sulfatase activating activity of a FGE or fusion proteins containing such proteins.

In all of the active type I sulfatase production methods described herein, the type I sulfatase, or the functional fragment of the type I sulfatase, as well as any of the proteins with the type I sulfatase activating activity of a FGE can be fused in frame to a leader or signal sequence. The leader or signal can be an exogenous or an endogenous leader or signal sequence. The leader or signal sequence can be, for example, the yeast Lip2pre leader sequence.

All the active type I sulfatase production methods described herein can further include introducing into the cell a nucleic acid encoding a polypeptide capable of effecting mannosyl phosphorylation (e.g., MNN4, PNO1, MNN6, or a functional fragment of such a polypeptide).

All the active type I sulfatase production methods described herein can also include introducing into the cell a nucleic acid encoding a mannosidase, or a functional fragment of a mannosidase, capable of hydrolyzing a terminal mannose-1-phospho-6-mannose moiety to a terminal phospho-6-mannose; this mannosidase can be, for example, the family 92 glycoside hydrolase CcMan5 from Cellulosimicrobium cellulans. The mannosidase, or the functional fragment of the mannosidase, can also be capable of removing a mannose residue bound by an alpha 1,2 linkage to the underlying mannose in the terminal mannose-1-phospho-6-mannose moiety; such a mannosidase can be a family 38 glycoside hydrolase selected from the group consisting of a Canavalia ensiformis (Jack Bean) mannosidase and Yarrowia lipolytica AMS1 mannosidase. Alternatively, or in addition, these methods can further include introducing into the cell a nucleic acid encoding a mannosidase, or a functional fragment of the mannosidase, that is capable of removing a mannose residue bound by an alpha 1,2 linkage to the underlying mannose in the terminal mannose-1-phospho-6-mannose moiety; this mannosidase can be the family 38 glycoside hydrolase Canavalia ensiformis (Jack Bean) mannosidase, the family 38 glycoside hydrolase Yarrowia lipolytica AMS1 mannosidase, the family 47 glycoside hydrolase Aspergillus satoi (AS) mannosidase, or the family 92 glycoside hydrolase Cellulosimicrobium cellulans CcMan4 mannosidase.

All of the active type I sulfatase production methods described herein can further include introducing into the cell a nucleic acid encoding a trafficking protein, or a functional fragment of the trafficking protein, which can direct any of the proteins with the type I sulfatase activating activity of a FGE to the endoplasmic reticulum (ER) of the cell. The trafficking protein can be Protein Disulfide Isomerase (PDI), Endoplasmic Reticulum Protein 44 (Erp44), or the inactive homolog of FGE in humans named SUMF2 (sulfatase modifying factor 2). The trafficking protein, or the functional fragment of the trafficking protein, can bind to the any of the proteins with the type I sulfatase activating activity of a FGE.

In all the active type I sulfatase production methods described herein, the fungal cell can be a yeast cell, e.g., a Yarrowia lipolytica cell, an Arxula adeninivorans cell, or a cell of another related species of dimorphic yeast. Alternatively, the yeast cell can be a Saccharomyces cerevisiae cell or a cell of a methylotrophic yeast (e.g., a cell of Pichia pastoris, Pichia methanolica, Ogataea minuta, or Hansenula polymorpha). Alternatively, in all the above methods, the fungal cell can be a cell of a filamentous fungus (e.g., Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowii, Aspergillus tamari, Aspergillus terreus, Aspergillus ustus, Aspergillus versicolor, Trichoderma, or Neurospora).

In any of the active type I sulfatase production methods described herein, the cell can include a deficiency in Outer Chain elongation (OCH1) protein 1 activity.

In all of the active type I sulfatase production methods described herein, coding sequences encoding type I sulfatase, or the functional fragment of the type I sulfatase coding sequence, any of the proteins with the type I sulfatase activating activity of a FGE, as well as other proteins (such as trafficking proteins, proteins capable of producing mannosyl phosphorylation, mannosidases, or functional fragments and variants of such proteins) can be under the control of a yeast (e.g., Yarrowia lipolytica, Arxula adeninivorans, or other related dimorphic yeast species) promoter for expression in a yeast cell. Each of the coding sequences can be under the control of the same yeast promoter, or the coding sequences can be under the control of different yeast promoters. For example, the yeast promoter can be hp4d or POX2.

In any of the active type I sulfatase production methods described herein, the coding sequences of the type I sulfatase, the functional fragment of the type I sulfatase, any of the proteins with the type I sulfatase activating activity of a FGE, as well as other proteins (such as trafficking proteins, proteins capable of producing mannosyl phosphorylation, mannosidases, or functional fragments and variants of such proteins) can be present as a single copy or as multiple copies, e.g., 2 copies. Each of the copies can be under the control of the same yeast promoter, or each of the copies can be under the control of different yeast promoters. For example, the yeast promoter for the first copy can be hp4d and the yeast promoter for the second copy can be POX2.

In all of the active type I sulfatase production methods described herein, the sulfatase can a human type I sulfatase. The type I sulfatase can be, for example, iduronate sulfatase (hIDS) or sulfamidase (SGSH).

In all of the three active type I sulfatase production methods described above, after step (b), the cell, or the progeny thereof, can be cultivated at high pO₂. The cell, or the progeny of the cell, can be cultivated at a pO₂ of, for example, 5%-40% (e.g., 10%, 15%, 20%, 25%, 30%, or 35%).

All of the active type I sulfatase production methods described herein can result in the production of a type I sulfatase, or a functional fragment of the type I sulfatase, in which greater than 10%, 20%, 30%, 40%, 50%, 60%, 70%, 80%, 90%, 95%, or 100% of the molecules of the type I sulfatase or functional fragment contain a formylglycine residue in the active site. It is to be understood that an activation of 100% is detected at a detection limit of 0.5% and therefore includes values from 99.5% to 100%.

In any of the active type I sulfatase production methods described herein, the protein with the type I sulfatase activity of a FGE (i) can include or be any of (a)-(f) as follows: (a) a mature wild type FGE polypeptide; (b) a functional fragment of a mature wild type FGE polypeptide comprising at least 50 (e.g., at least: 60; 70; 80; 90; 100; 125; 150; 175; 200; 225; 250; 275; 300; 325; 350; 400; 450; 500; or more) consecutive amino acids of the mature wild type FGE; (c) a polypeptide with at least 80% (e.g., at least: 85%; 88%; 90%; 92%; 95%; 98%; 99%; or 99.5%) identity to (a); (d) a polypeptide with at least 90% (e.g., at least: 92%; 95%; 98%; 99%; or 99.5%) identity to (b); (e) (a) but with no more than 10 (e.g., no more than 8; 7; 6; 5; 4; 3; 2; or 1) conservative substitution(s); and (f) (b) but with no more than 5 (e.g., no more than 4; 3; 2; or 1) conservative substitutions(s), where the mature wild type FGE polypeptide is a mature wild type Columba livia FGE. Moreover, the protein with the type I sulfatase activity of a FGE can further include a yeast MNS1 transmembrane anchor polypeptide. The protein with the type I sulfatase activating activity of a FGE can have or contain the amino acid sequence set forth in SEQ ID NO: 63.

The present document also features an active type I sulfatase, or a functional fragment of an active type I sulfatase, produced by the any of the active type I sulfatase production methods described herein. The document also provides a method of treating a subject having, or suspected of having, a disorder treatable with a type I sulfatase, the method comprising administering to the subject the active type I sulfatase, or a functional fragment of the type I sulfatase, produced by any of the active type I sulfatase production methods described herein. The disorder can be, for example, a lysosomal storage disorder or a disease of some other subcellular compartment or organelle (e.g., the Golgi or microsomes). The disorder can be, without limitation, metachromatic leukodystrophy, Hunter disease, Sanfilippo disease A & D, Morquio disease A, Maroteaux-Lamy disease, X-linked ichthyosis, Chondrodysplasia Punctata 1, and multiple sulfatase deficiency (MSD). Moreover, in these methods, the subject can be a human.

The document also features an isolated fungal cell that contains a nucleic acid encoding a the protein with the type I sulfatase activity of a FGE. The protein with the type I sulfatase activity of a FGE (i) can include or be any of (a)-(f) as follows: (a) a mature wild type FGE polypeptide; (b) a functional fragment of a mature wild type FGE polypeptide comprising at least 50 (e.g., at least: 60; 70; 80; 90; 100; 125; 150; 175; 200; 225; 250; 275; 300; 325; 350; 400; 450; 500; or more) consecutive amino acids of the mature wild type FGE; (c) a polypeptide with at least 80% (e.g., at least: 85%; 88%; 90%; 92%; 95%; 98%; 99%; or 99.5%) identity to (a); (d) a polypeptide with at least 90% (e.g., at least: 92%; 95%; 98%; 99%; or 99.5%) identity to (b); (e) (a) but with no more than 10 (e.g., no more than 8; 7; 6; 5; 4; 3; 2; or 1) conservative substitution(s); and (f) (b) but with no more than 5 (e.g., no more than 4; 3; 2; or 1) conservative substitutions(s) This fungal cell can also contain a nucleic acid encoding a type I sulfatase, a functional fragment of a type I sulfatase or a fusion protein containing a type I sulfatase or a functional fragment thereof. The encoded type I sulfatase, or the encoded functional fragment of the type I sulfatase, without the action of an activating factor on it, is an inactive form.

In all fungal cells containing a nucleic acid encoding an FGE, the FGE can be any of the following mature wild type proteins (or functional fragments thereof) and variants (listed above) of either: mature wild type protein SCO7548; mature wild type protein Rv0712; mature wild type sulfatase modifying factor 1; mature wild type C-alpha-formylglycine-generating enzyme; or mature wild type sulfatase-modifying factor 1. Also useful are fungal cells producing fusion proteins containing any of the mature wild type proteins, functional fragments, and variants of both. Moreover, the FGE can be a prokaryotic FGE (e.g., a FGE from Mycobacterium tuberculosis or Streptomyces coelicolor). Alternatively, the FGE can be a eukaryotic FGE (e.g., a FGE of Homo sapiens, Bos taurus, Hemicentrotus pulcherrimus, Tupaia chinensis, Monodelphis domestica, Gallus gallus, Dendroctonus ponderosa, or Columba livia).

In addition, in any of the fungal cells of the disclosure, any of the proteins with the type I sulfatase activating activity of a FGE, fusions containing such proteins, can further include a ER targeting motif such as HDEL (SEQ ID NO: 1), KDEL (SEQ ID NO: 3), DDEL (SEQ ID NO: 4), RDEL (SEQ ID NO: 33), a yeast MNS1 transmembrane anchor polypeptide (such as the Yarrowia lipolytica MNS1 transmembrane anchor polypeptide), oyeast WBP1 transmembrane anchor polypeptide (such as the Yarrowia lipolytica WBP1 transmembrane anchor polypeptide), or the transmembrane parts of Secretory-12 (SEC12), Glucosidase-1 (GLS1), or STaurosporine Temperature Sensitive-3 (STT3). The ER targeting motif can be fused to the N-terminus or the C-terminus of any of the proteins with the type I sulfatase activating activity of a FGE, or fusion proteins containing such proteins

In all of the fungal cells of this disclosure, the type I sulfatase, or the functional fragment of the type I sulfatase, as well as any of the proteins with the type I sulfatase activating activity of a FGE of the can be fused in frame to a leader or signal sequence. The leader or signal can be an exogenous or an endogenous leader or signal sequence. The leader or signal sequence can be, for example, the Lip2pre leader sequence.

All the fungal cells of this disclosure can further include a nucleic acid encoding a polypeptide capable of effecting mannosyl phosphorylation (e.g., MNN4, PNO1, MNN6, or a functional fragment of such a polypeptide).

In addition, all the fungal cells of this disclosure can also contain a nucleic acid encoding a mannosidase, or a functional fragment of a mannosidase, capable of hydrolyzing a terminal mannose-1-phospho-6-mannose moiety to a terminal phospho-6-mannose; this mannosidase can be, for example, the family 92 glycoside hydrolase CcMan5 from Cellulosimicrobium cellulans. The mannosidase, or the functional fragment of the mannosidase, can also be capable of removing a mannose residue bound by an alpha 1,2 linkage to the underlying mannose in the terminal mannose-1-phospho-6-mannose moiety; such a mannosidase can be a family 38 glycoside hydrolase selected from the group consisting of a Canavalia ensiformis (Jack Bean) mannosidase and Yarrowia lipolytica AMS1 mannosidase. Alternatively, or in addition, the fungal cells can further include a nucleic acid encoding a mannosidase, or a functional fragment of the mannosidase, that is capable of removing a mannose residue bound by an alpha 1,2 linkage to the underlying mannose in the terminal mannose-1-phospho-6-mannose moiety; this mannosidase can be the family 38 glycoside hydrolase Canavalia ensiformis (Jack Bean) mannosidase, the family 38 glycoside hydrolase Yarrowia lipolytica AMS1 mannosidase, the family 47 glycoside hydrolase Aspergillus satoi (AS) mannosidase, or the family 92 glycoside hydrolase Cellulosimicrobium cellulans CcMan4 mannosidase.

Furthermore, all of the fungal cells of this disclosure can also include a nucleic acid encoding a trafficking protein, or a functional fragment of the trafficking protein, which can direct any of the proteins with the type I sulfatase activating activity of a FGE to the endoplasmic reticulum (ER) of the cell. The trafficking protein can be Protein Disulfide Isomerase (PDI), Endoplasmic Reticulum Protein 44 (Erp44), or the inactive homolog of FGE in humans named SUMF2. The trafficking protein, or the functional fragment of the trafficking protein, can bind to the any of the proteins with the type I sulfatase activating activity of a FGE.

The fungal cell of this disclosure can be a yeast cell, e.g., a Yarrowia lipolytica cell, an Arxula adeninivorans cell or a cell of another related species of dimorphic yeast. Alternatively, the yeast cell can be a Saccharomyces cerevisiae cell or a cell of a methylotrophic yeast (e.g., a cell of Pichia pastoris, Pichia methanolica, Ogataea minuta, or Hansenula polymorpha). Alternatively, in all the above methods, the fungal cell can be a cell of a filamentous fungus (e.g., Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowii, Aspergillus tamari, Aspergillus terreus, Aspergillus ustus, Aspergillus versicolor, Trichoderma, or Neurospora).

In any of the fungal cells of this disclosure, the cell can include a deficiency in Outer Chain elongation (OCH1) protein 1 activity.

In all of the fungal cells of this disclosure, coding sequences encoding type I sulfatase, or the functional fragment of the type I sulfatase coding sequence, any of the proteins with the type I sulfatase activating activity of a FGE, as well as other proteins (such as trafficking proteins, proteins capable of producing mannosyl phosphorylation, mannosidases, or functional fragments and variants of such proteins) can be under the control of a yeast (e.g., Yarrowia lipolytica, Arxula adeninivorans, or other related dimorphic yeast species) promoter for expression in a yeast cell. Each of the coding sequences can be under the control of the same yeast promoter, or the coding sequences can be under the control of different yeast promoters. For example, the yeast promoter can be hp4d or POX2. Moreover, any can be present as a single copy or as multiple copies, e.g. 2 copies. Each of the copies can be under the control of the same yeast promoter, or each of the copies can be under the control of different yeast promoters. For example, the yeast promoter for the first copy can be hp4d and the yeast promoter for the second copy can be POX2.

In all of the fungal cells of this disclosure, the sulfatase can a human type I sulfatase. The type I sulfatase can be, for example, iduronate sulfatase (hIDS) or sulfamidase (SGSH).

In any of the fungal cells of this disclosure, the protein with the type I sulfatase activity of a FGE (i) can include or be any of (a)-(f) as follows: (a) a mature wild type FGE polypeptide; (b) a functional fragment of a mature wild type FGE polypeptide comprising at least 50 (e.g., at least: 60; 70; 80; 90; 100; 125; 150; 175; 200; 225; 250; 275; 300; 325; 350; 400; 450; 500; or more) consecutive amino acids of the mature wild type FGE; (c) a polypeptide with at least 80% (e.g., at least: 85%; 88%; 90%; 92%; 95%; 98%; 99%; or 99.5%) identity to (a); (d) a polypeptide with at least 90% (e.g., at least: 92%; 95%; 98%; 99%; or 99.5%) identity to (b); (e) (a) but with no more than 10 (e.g., no more than 8; 7; 6; 5; 4; 3; 2; or 1) conservative substitution(s); and (f) (b) but with no more than 5 (e.g., no more than 4; 3; 2; or 1) conservative substitutions(s), where the mature wild type FGE polypeptide is a mature wild type Columba livia FGE. Moreover, the protein with the type I sulfatase activity of a FGE can further include a yeast MNS1 transmembrane anchor polypeptide. The protein with the type I sulfatase activating activity of a FGE can have or contain the amino acid sequence set forth in SEQ ID NO: 63.

The document also provides a substantially pure culture comprising fungal cells which are genetically engineered to comprise a protein with the type I sulfatase activating activity of a FGE. The fungal cells further comprising a nucleic acid encoding a type I sulfatase, or a functional fragment thereof, wherein the encoded type I sulfatase, or functional fragment thereof, without the action of an activating factor on it, is an inactive form. The fungal cells of the culture can have any of the attributes, characteristics, and properties of the fungal cells described above and can express any of the wild type proteins, functional fragments of such proteins, and variants described herein.

In any of the above methods or fungal cells, the mature wild type FGE can be: (i) a mature wild type FGE of Hemicentrotus pulcherrimus having the amino acid sequence set forth in SEQ ID NO: 13, a mature wild type FGE of Gallus gallus having the amino acid sequence set forth in SEQ ID NO: 47, a mature wild type FGE of Dendroctonus ponderosa having the amino acid sequence set forth in SEQ ID NO: 49, or a mature wild type FGE of Columba livia having the amino acid sequence set forth in SEQ ID NO: 51; (ii) a functional mature FGE having an amino acid sequence that is at least 80% identical to any one of the amino acid sequences of (i).

Moreover, in any of the above methods or fungal cells, the protein with the type I sulfatase activating activity of a FGE can be encoded by a nucleotide sequence having: (i) the nucleic acid sequence set out in any one of SEQ ID NOs: 14, 48, 50 or 52; or (ii) a nucleic acid sequence that is at least 80% identical to any one of the nucleic acid sequences of (i) and encodes a functional FGE; or (iii) a nucleic acid sequence that hybridizes to a complement of any one of the nucleic acid sequences of (i) under high stringency and encodes a functional FGE.

Unless otherwise defined, all technical and scientific terms used herein have the same meaning as commonly understood by one of ordinary skill in the art to which the embodiments of this document belong. Although methods and materials similar or equivalent to those described herein can be used in the practice or testing of these embodiments, the exemplary methods and materials are described below. All publications, patent applications, patents, Genbank® Accession Nos, and other references mentioned herein are incorporated by reference in their entirety. In case of conflict, the present application, including definitions, will control. The materials, methods, and examples are illustrative only and not intended to be limiting.

Other features and advantages of the materials and methods recited in this disclosure, e.g., methods of activating type I sulfatases or functional fragments thereof, will be apparent from the following detailed description, and from the claims.

### DESCRIPTION OF DRAWINGS

FIG. 1A is a schematic representation of the recombinant Formylglycine Generating Enzyme (rFGE) fusion proteins produced by genetically engineered cells described herein and how their native leader sequence (FGE-LS) is replaced with the LIP2 pre leader (signal) sequence. Each fusion protein contains, N-terminus to C-terminus, the Lip2 pre leader sequence (LIP2pre), a mature FGE (FGE; e.g., mature Bos taurus FGE), a hexahistidine tag (6HIS), and a HDEL (SEQ ID NO: 1) tetrapeptide. FIG. 1B is a depiction of the amino acid sequence (SEQ ID NO: 32) of a fusion protein as described for FIG. 1A in which the mature FGE is Bos taurus FGE (BtFGE). LIP2pre is in bold italics and underlined, the mature BtFGE is in plain bold text, the 6HIS is in plain text and underlined, and the HDEL is in plain italics text.

FIGS. 2A, 2B, and 2C are photographs of sodium dodecyl sulfate polyacrylamide gel electrophoresis (SDS-PAGE) analyses detecting recombinant human iduronate-2 sulfatase (rhIDS) as expressed in Y. lipolytica at 28° C. The gel depicted in FIG. 2A shows expression of rIDS from T146 (OXYY1828; BtFGE) clones A-F in lanes 1-6 and T147 (OXYY1831; ScFGE) clones A, B and C in lane 7-9, respectively. The gel depicted in FIG. 2B shows expression of rhIDS from T147 (OXYY1831; ScFGE) clones D-F in lanes 11-13 and from T148 (OXYY1801; HpFGE) clones A-F in lanes 15-20. The gel depicted in FIG. 2C shows expression of rhIDS from T126 (OXYY1827; hFGE) clones A-D in lanes 21-24. Molecular weight markers are shown in lanes 10, 14 and 26 of FIGS. 2A, 2B, and 2C, respectively. Lane 27 contains ELAPRASE® (idursulfase) which is a commercial human IDS preparation. The arrows in the photographs indicate detection of rhIDS protein.

FIGS. 3A, 3B, and 3C are digital images of a chemiluminiscent reaction showing the Western blot analysis of rFGE under reducing conditions. The image depicted in FIG. 3A shows expression of rFGE from T146 (OXYY1828; BtFGE) clones A and B at 28° C. (lanes 1 and 2) and at 20° C. (lanes 3 and 4) in lanes 1-4, and from T147 (OXYY1831; ScFGE) clones A and B at 28° C. (lanes 5 and 6) and 20° C. (lanes 7 and 8) in lanes 5-8. The image depicted in FIG. 3B shows expression of rFGE from T148 (OXYY1801; HpGFE) clones A and B at 28° C. (lanes 11 and 12) and at 20° C. (lanes 13 and 14) in lanes 11-14, and from T153 (OXYY1802; MtFGE) clones A and B at 28° C. (lanes 15 and 16) and 20° C. (lanes 17 and 18) in lanes 15-18. FGE expression for T126 (OXYY1827; hFGE) at 28° C. and 20° C. is shown in lane 9 of FIG. 3A and lane 19 of FIG. 3B respectively. FIG. 3C shows expression of rFGE from a clone of T148 (OXYY1801; HpGFE) grown at 28° C. in lane 21; a clone of T153 (OXYY1802; MtFGE) grown at 28° C. in lane 22; a clone of T148 (OXYY1801) grown at 20° C. in lane 23; a clone of T153 (OXYY1802; MtFGE) grown at 20° C. in lane 24; a clone of T161 (OXYY1798, BtFGE) grown at 28° C. in lane 25; a clone of T156 (OXYY1803; BtFGE and hPDI) grown at 28° C. in lane 26; and a clone of T146 (OXYY1828; BtFGE) grown at 28° C. in lane 27. Molecular weight markers are shown in lanes 10, 20, and 28 of FIGS. 3A, 3B, and 3C respectively. The arrows in the photographs indicate detection of rFGE protein.

FIG. 4 is a digital image of a chemiluminiscent reaction displaying the Western blot analysis of rFGE under reducing and non-reducing conditions. Expression of rFGE from T126 (OXYY1827; hFGE) clones A and B at 28° C. (lanes 1 and 2) and at 20° C. (lanes 3 and 4) under reducing conditions are shown in lanes 1-4 and under non-reducing conditions in lanes 6-9. Molecular weight markers are shown in lane 5.

FIGS. 5A and 5B are a photograph of an SDS-PAGE analysis (FIG. 5A) and a digital image of a chemiluminiscent reaction of a Western blot analysis (FIG. 5B) showing rhIDS expression in the presence of FGE co-expression in strains T146 (OXYY1828), T147 (OXYY1831), T148 (OXYY1801) and T153 (OXYY1802) which co-express Bos taurus FGE (BtFGE), Streptomyces coelicolor FGE (ScFGE), Hemicentrotus pulcherrimus (HpFGE), and Mycobacterium tuberculosis FGE (MtFGE), respectively. The expression of each clone was analyzed at 4 timepoints. The arrows in the images indicate detection of rIDS protein. Molecular weight markers are shown the left-most lane of the photograph and the digital image. ELAPRASE® was included in the indicated lanes.

FIG. 6 is a bar graph depicting the percentages of total rhIDS produced at 28° C. and 20° C. in heterologous Y. lipolytica cells co-expressing rIDS and rFGE of different origins that are functional.

FIG. 7A is a diagrammatic representation of the rFGE and Yarrowia lipolytica MNS1 mannosidase anchorage domain containing fusion proteins described in Example 10. Each fusion protein contains, N-terminus to C-terminus, amino acids 1-163 of MNS1 (SEQ ID NO:26), a mature FGE (e.g., BtFGE), and a hexahistidine (6HIS) tag; FIG. 7B is a diagrammatic representation of the rFGE and Yarrowia lipolytica WBP1 oligosaccharyl transferase anchorage domain containing fusion proteins described in Example 11. Each fusion protein contains, N-terminus to C-terminus, the Lip2 signal sequence, a hexahistidine (6HIS) tag, a mature FGE (e.g., BtFGE), and the C-terminal 118 amino acids (amino acids 400-505 of XP_502492.1) of Yarrowia lipolytica WBP1 (SEQ ID NO:28); FIG. 7C is a diagrammatic representation of the chimeric protein consisting of of the N-terminal end of BtFGE (amino acids 32-104 of NP_001069544, fused to the C-terminal end of HpFGE (amino acids 144-423 of BAJ83907) described in Example 12. The Lip2 leader was fused to the N-terminal end of the chimeric coding sequence and at the C-terminus a 6HIS tag was added, followed by the HDEL tetrapeptide.

FIG. 8A is a digital image of a chemiluminiscent reaction displaying the Western blot analysis (by Western blot with a rabbit anti-human IDS antiserum) for expression of rhIDS from strains co-expressing rhIDS (1 copy, POX2 driven) and rFGE (1 copy POX2 driven and 1 copy Hp4d driven) grown under fed-batch fermentation. The Y. lipolytica-produced IDS is visible at an approximate MW of 76 kDa. The supernatant was analyzed for six rIDS expressing strains at the endpoint of the fermentation. Lane 1 is the MW Marker; lane 2 is ChFGE (the chimeric protein described in Example 12) co-expressed at 20° C.; lane 3 is ChFGE co-expressed at 28° C.; lane 6 is BtFGE-WBP1 co-expression; lane 7 is BtFGE-MNS1 co-expression; and lanes 8-9 are the control strains co-expressing BtFGE-HDEL (1 copy, POX2 driven). FIG. 8B is a digital image of a chemiluminiscent reaction displaying the Western blot analysis for expression of rFGE using anti-his antibody (A00186-100, Genscript). The contents in each lane correspond to those in FIG. 8A.

### DETAILED DESCRIPTION

Type I sulfatases require a unique co- or post-translational amino acid modification in the active center of the enzyme to enable their activation, specifically, a cysteine in the active site is oxidized to the aldehyde-containing a Ca-Formylglycine residue. In humans, a single enzyme, sulfatase modifying factor-1 (SUMF1) or formylglycine generating enzyme (FGE) is responsible for activation of all type I sulfatases. Inactivity of FGE leads to the production of catalytically inactive type I sulfatases, the cause of a rare but fatal lysosomal storage disease called Multiple Sulfatase Deficiency (MSD) (Dierks et al (2003), Cell, 113, 435-444).

The formylglycine (FGly) residue of an activated type I sulfatase is located in a 13 amino acid consensus sequence called the sulfatase motif. Formylglycine can be generated from a cysteine residue within the core motif [CX(P/A)XR] or a serine residue within the core motif [S/CXPXR]. Each ‘X’ in this core motif represents any amino acid. In eukaryotic organisms, the conversion starting from cysteine is the only known route. Conversion starting from serine is predominantly found in anaerobic bacteria as the conversion of the thiol group of cysteine to an aldehyde group catalyzed by FGly-generating enzyme is oxygen-dependent. The mechanism by which FGly is formed by FGE is still unknown. It has been determined that the structure of FGE-substrate complexes includes pentamer and heptamer peptides that mimic the substrate. It was shown that the peptides isolate a cavity that can serve as a binding site for molecular oxygen (Roeser et al (2006), Proceedings of the National Academy of Sciences of the United States of America, 103, 81-86). The inactive homolog of FGE in humans, SUMF2 is also a trafficking protein.

The enzyme acts on the newly synthesized type I sulfatase when it is entering the endoplasmic reticulum (ER) and when it is still in its unfolded form. Once the nascent type I sulfatase is fully folded, the target cysteine becomes incorporated in the active site cleft where it is inaccessible for modification by FGE, resulting in the production of an inactive type I sulfatase. In humans, the FGE lacks a C-terminal ER retrieval signal and is also dependent on interaction with other proteins for its correct localization. Both Protein Disulfide Isomerase (PDI) and Endoplasmic Reticulum Protein (Erp44), two ER resident proteins, have been shown to interact with FGE and are thought to be involved in the control of FGE trafficking and functioning via non-covalent hetero-oligomeric interaction (Fraldi et al (2008), Human molecular genetics, 17, 2610-2621 and Mariappan et al (2008), The Journal of Biological Chemistry, 283, 6375-6383).

The interaction is likely to occur through the N-terminal extension of FGE that confers not only ER localization to FGE but is also indispensable for its in vivo catalytic activity.

In humans, a paralog of FGE has also been identified as the SUMF2 gene product. It is catalytically inactive and has substantial expression levels (Gande et al (2008), The FEBS Journal, 275, 1118-1130). There is evidence that FGE and its paralog act in concert by forming heterodimers. Also, in vivo the paralog seems to contact nascent type I sulfatases hereby forming ternary complexes with FGE (Zito et al (2005), EMBO Reports, 6, 655-660). The human paralog is retrieved to the ER through a C-terminal KDEL-like signal, but does not seem to act as a standalone retention factor for ER localization of FGE. Conferring ER localization of human FGE through fusion for the HDEL (SEQ ID NO: 1; corresponding nucleic acid sequence set forth in SEQ ID NO: 2) tetrapeptide has been shown to be sufficient and effective. An alternative approach to obtain correct localization of the FGE protein to the ER is to fuse a transmembrane anchor to the FGE. For example, the transmembrane anchor of a yeast α-1,2-mannosidase (MNS1) or a yeast wheat germ agglutinin-binding protein (WBP1) such as those of Saccharomyces cerevisiae or Yarrowia lipolytica can be used. Y. lipoytica MNS1 has Accession No: XP_502939.1 and Yarrowia lipolytica WBP1 has Accession No.: XP_502492.1.

Human FGE (hFGE) is encoded by the SUMF1 gene. The immature protein is a protein of 374 residues, including a signal sequence of 33 amino acids (SEQ ID NO: 23) which induces the translocation of the protein into the ER. The amino acid sequence of mature hFGE is designated SEQ ID NO:9. A single N-glycosylation site is also present at Asn141 (residue number is that of the immature hFGE protein). The folding of the protein shows remarkably little secondary structure (Roeser et al (2006), Proceedings of the National Academy of Sciences of the United States of America, 103, 81-86). Human FGE is a compact monomeric molecule that is stabilized by two intramolecular disulfide bridges and two calcium molecules. It has a binding groove for the CXPXR substrate peptide which has two cysteines, Cys₃₃₆ and Cys₃₄₁ (residue numbers are those of the immature hFGE protein), involved in the formation of FGly, as discussed above. SUMF1 homologues have been identified across a large variety of species and are highly conserved (Sardiello et al (2005), Human Molecular Genetics, 14, 3203-3217). However, thus far, no FGE homologues have been identified in Yarrowia lypolytica or other fungal species despite the presence of a type I sulfatase gene (Sardiello et al (2005), Human Molecular Genetics, 14, 3203-3217).

In eukaryotes, the minimal canonical sequence CxPxR (where each x is any amino acid) in the active site of type I sulfatases is recognized by an FGly-generating enzyme, which catalyzes the oxidation of the cysteine residue to an aldehyde-bearing Ca-formylglycine residue. This reaction is a multistep redox reaction that involves disulfide bridge formation and requires molecular oxygen and a reducing agent but does not require a cofactor or a metal ion (Roeser et al (2006), Proceedings of the National Academy of Sciences of the United States of America, 103, 81-86). This conversion from cysteine to formylglycine is an activation step that is essential for the type I sulfatase activity of the type I sulfatases.

In general, this document discloses methods and materials for the production and isolation of catalytically active type I sulfatases in recombinant fungal cells. Also provided are methods to produce active type I sulfatases in the presence of FGEs and, optionally, other polypeptides, such as trafficking molecules, mannosidases, and polypeptides that effect mannose phosphorylation. The utilization of FGEs from varying sources is included.

Also included in this document are methods and materials for hydrolyzing a terminal mannose-1-phospho-6-mannose linkage or moiety on an N-glycan on a type I sulfatase to phospho-6-mannose (also referred to as “mannose-6-phosphate” herein) (“uncapping”) and hydrolyzing a terminal alpha-1,2 mannose, alpha-1,3 mannose and/or alpha-1,6 mannose linkage or moiety of such a phosphate-containing N-glycan (“demannosylating”). Also provided are methods of facilitating uptake of a glycoprotein (e.g., an activated type I sulfatase) by a mammalian cell as both uncapping and demannosylation (either by separate enzymes or a single enzyme) are required to achieve mammalian cellular uptake of glycoproteins via mannose-6-phosphate receptors. For further details on these methods, see for example, PCT application PCT/IB2011/002770 or U.S. Application Publication No. US2013/0267473-A1, the disclosures of which are incorporated herein by reference in their entirety.

The methods and materials described herein are useful for making agents for the treatment of any condition in which it is desired to administer an activated type I sulfatase (e.g., an activated type I sulfatase, or a functional fragment thereof) to a subject (e.g., a human patient with the condition). They are particularly useful for producing agents for treating subjects with lysosomal storage disorders (LSDs) in which one or more type I sulfatases are absent, inactive, or insufficiently active. Moreover, they can be used to treat MSD in which afflicted subjects produce catalytically inactive FGE. LSDs are a diverse group of hereditary metabolic disorders characterized by the accumulation of storage products in the lysosomes due to impaired activity of catabolic enzymes involved in their degradation. The build-up of storage products leads to cell dysfunction and progressive clinical manifestations. Deficiencies in catabolic enzymes can be corrected by enzyme replacement therapy (ERT), provided that the administered enzyme can be targeted to the lysosomes of the diseased cells. Lysosomal enzymes typically are glycoproteins that are synthesized in the ER, transported via the secretory pathway to the Golgi, and then recruited to the lysosomes. Using the methods and materials described herein, a microbe-based production process can be used to obtain therapeutic type I sulfatases. In some embodiments these type I sulfatases have demannosylated phosphorylated N-glycans. Thus, the methods and materials described herein are useful for preparing type I sulfatases for the treatment of disorders such as, for example, LSDs. Relevant disorders include, without limitation, metachromatic leukodystrophy (arylsulfatase A), Hunter disease (iduronate 2-sulfatase), Sanfilippo disease A (N-sulfoglucosamine sulfohydrolase) & D (N-acetylglucosamine-6-sulfatas), Morquio disease A (Galactosamine-6-sulfatase), Maroteaux-Lamy disease (arylsulfatase B), X-linked ichthyosis (steroid sulfatase), Chondrodysplasia Punctata 1 (arylsulfatase E), and MSD. For other relevant disorders, see, for example, Diez-Roux et al. (2005), Annu Rev Genomics Hum Genet, 6, 355-379, the disclosure of which is incorporated herein by reference in its entirety.

As used herein, a type I sulfatase that is in an “active form” is one that has more than 5% (e.g., more than: 7.5%; 10%; 20%; 30%; 40%; 50%; 60%; 70%; 80%; 90%; 100%; or even more) of the type I sulfatase activity of a wild-type type I sulfatase obtained from a mammalian cell with a normal level of FGE with normal activity and with wild type expression levels of sulfatases and with the specificity of the relevant wild type type I sulfatase.

As used herein, the terms “inactive type I sulfatase”, “type I sulfatase in an inactive form”, “type I sulfatase that is not in an active form”, “type I sulfatase that is not active”, and similar terms refer to a type I sulfatase that has no more than 5% (e.g., no more than: 2.5%; 1.0%; 0.1%; 0.01%; or none) of the type I sulfatase activity of a wild-type type I sulfatase obtained from a cell with a normal level of FGE with normal activity and with wild type expression levels of sulfatases and with the specificity for the relevant wild type type I sulfatase. This document provides methods that include the use of nucleic acids encoding type I sulfatases and FGEs.

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

“Polypeptide” and “protein” are used interchangeably herein and mean any peptide-linked chain of amino acids, regardless of length or post-translational modification. Typically, a polypeptide described herein (e.g., a type I sulfatase or an FGE) is isolated when it constitutes at least 60%, by weight, of the total protein in a preparation, e.g., 60% of the total protein in a sample. In some embodiments, a polypeptide described herein consists of at least 75%, at least 90%, or at least 99%, by weight, of the total protein in a preparation.

The term “active site” is a defined region of an enzyme where a substrate binds to subsequently undergo a chemical reaction. The active site is the region in which the chemical reaction occurs. The active site of an enzyme can be found in a cleft or pocket that can be lined with amino acid residues that participates in recognition of a substrate. Residues that directly participate in a catalytic reaction mechanism of a substrate are in the active site. In certain instances, as described herein, a residue of the enzyme requires post translational modification. In some instances, the residue is in the active site of the protein (i.e., formylglycine in the active site of type I sulfatase). Substrates bind to the active site of the enzyme through chemical interactions selected from a group comprising hydrogen bonds, hydrophobic interactions, electrostatic interactions, van de Waal's forces, and temporary covalent interactions. In further embodiments, a combination of these to form the enzyme-substrate complex can be used. The active site can modify the reaction mechanism to change the activation energy of the reaction involving the substrate. The consensus active site of an enzyme or the consensus sequence within an active site is the highly homologous region of conserved residues which are shared by a family of proteins (i.e. enzymes).

The term “activation step”, as used herein with respect to the production of a type I sulfatase in an active form, or a functional fragment thereof, refers to an intracellular process that occurs before, during, or after the intracellular folding of the type I sulfatase polypeptide, or the functional fragment thereof, that results in the type I sulfatase polypeptide, or the functional fragment thereof, after it is fully folded, being in an active form. Such an activation step can be, but is not necessarily, effected by an activating factor.

As used herein, the term “activating factor” refers to an enzyme (e.g, an FGE), or a functional fragment thereof, that, before, during or after the intracellular folding of a type I sulfatase, or a functional fragment thereof, acts on the type I sulfatase, or functional fragment thereof, such that the fully folded type I sulfatase, or fully folded functional fragment thereof, is in an active form.

As used herein, the term “at an increased level”, when used with respect to the production of a type I sulfatase in an active form, or a functional fragment thereof, in a fungal cell expressing an exogenous nucleic acid encoding an activating factor (e.g., an FGE), refers to the increased level of the type I sulfatase in an active form, or the functional fragment thereof, produced in the fungal cell as compared to the level produced by a control fungal cell not expressing an exogenous nucleic acid encoding an activating factor.

An “isolated nucleic acid” refers to a nucleic acid that is separated from other nucleic acid molecules that are present in a naturally-occurring genome, including nucleic acids that normally flank one or both sides of the nucleic acid in a naturally-occurring genome (e.g. a yeast genome). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

The term “functional fragment” as used herein refers to a peptide fragment of a protein that is shorter (in terms of amino acid number) than the corresponding mature, full-length, wild-type protein and has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein (i.e., be composed of consecutive amino acids of the protein) wherein the region has functional activity. The term “functional fragment” also refers to a peptide fragment of a protein that can be made active or have the ability to be activated by means of an activation step to have the activity of the corresponding activated mature, full-length, wild-type protein. The functional fragment can contain the activation site of type I sulfatase in an active form or not in an active form; the latter type of functional fragment would have the ability to be activated by the action of an FGE. The consensus amino acid sequence of the type I sulfatase active site is described herein. Candidate functional fragments of type I sulfatases can therefore be produced by one skilled in the art using well established methods. Their activity can be confirmed by well-established methods such as those described in the working examples disclosed here. Functional fragments will generally be at least 20 (e.g., at least: 30; 40; 60; 70; 80; 90; 100; 125; 150; 175; 200; 225; 250; 275; 300; 325; 350; 400; 450; 500; or more) amino acids long.

A “functional mature FGE” as used herein with reference to a variant mature FGE polypeptides or a variant nucleic acid encoding a variant FGE polypeptide has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type polypeptide.

This document also provides (i) functional variants of the proteins used in the methods of the document and (ii) functional variants of the functional fragments described above. Functional variants of the proteins and functional fragments can contain additions, deletions, or substitutions relative to the corresponding wild-type sequences. Proteins with substitutions will generally have not more than 50 (e.g., not more than one, two, three, four, five, six, seven, eight, nine, ten, 12, 15, 20, 25, 30, 35, 40, or 50) conservative amino acid substitutions. This applies to any of the above-mentioned proteins and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids.

Substitutions and deletions in type I sulfatases will preferably not be in the active site. In particular, a cysteine residue that is converted to a formylglycine upon activation should not be substituted or deleted.

Additions (addition variants) include fusion proteins containing: (a) any of the above-described proteins or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine (SEQ ID NO: 7; corresponding nucleic acid sequence set forth in SEQ ID NO: 8)), hemagluttanin (HA), glutathione-S-transferase (GST), or maltosebinding protein (MBP)). Heterologous sequences also can be proteins useful as diagnostic or detectable markers, for example, luciferase, green fluorescent protein (GFP), or chloramphenicol acetyl transferase (CAT). In some embodiments, the fusion protein contains a signal sequence or leader sequence from another protein. In certain host cells (e.g., yeast host cells), expression and/or secretion of the target protein can be increased through use of a heterologous signal sequence. For example, the signal (leader) sequence may be the Lip2pre sequence. In some embodiments, the fusion protein can contain a carrier (e.g., keyhole limpet hemocyanin (KLH)) useful, e.g., in eliciting an immune response for antibody generation) or ER or Golgi apparatus retention signals. Soluble proteins that reside in the lumen of the ER are known to have at their C terminus, inter alia, the tetrapeptides KDEL (SEQ ID NO: 3) or HDEL (SEQ ID NO: 1; corresponding nucleic acid sequence set forth in SEQ ID NO: 2). These tetrapeptides, and others such as DDEL (SEQ ID NO:4) and RDEL (SEQ ID NO: 33), function as retrieval motifs essential for the precise sorting of these proteins along the secretory pathway. Their presence on the terminal end of a luminal protein signals trafficking to the ER. Additional retention signals that may be used in a fusion protein include transmembrane anchors such the transmembrane anchors of yeast ER/Golgi residing proteins (e.g., S. cervisiae or Y. lipolytica MNS1 or WBP1). The amino acid sequence of the transmembrane anchor polypeptide of Yarrowia lipolytica MNS1 is designated SEQ ID NO: 26 (the corresponding nucleic acid sequence is set forth in SEQ ID NO: 27), and the amino acid sequence of the transmembrane anchor polypeptide of Yarrowia lipolytica WBP1 is designated SEQ ID NO: 28 (the corresponding nucleic acid sequence set is forth in SEQ ID NO: 29). Heterologous sequences can be of varying length and in some cases can be a longer sequences than the full-length target proteins to which the heterologous sequences are attached.

As used herein, the term “wild-type” as applied to a nucleic acid or polypeptide refers to a nucleic acid or a polypeptide that occurs in, or is produced by, respectively, a biological organism as that biological organism exists in nature.

The term “exogenous” as used herein with reference to a nucleic acid (or a protein) and a host cell refers to (a) a nucleic acid that does not occur in (and cannot be obtained from) a cell of that particular type as found in nature or (b) a protein encoded by such a nucleic acid. Thus, a non-naturally-occurring nucleic acid is considered to be exogenous to a host cell once in the host cell. It is important to note that non-naturally-occurring nucleic acids can contain nucleic acid subsequences or fragments of nucleic acid sequences that are found in nature provided that the nucleic acid as a whole does not exist in nature. For example, a nucleic acid molecule containing a genomic DNA sequence within an expression vector is nonnaturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host cell. For example, an entire chromosome isolated from a cell of yeast x is an exogenous nucleic acid with respect to a cell of yeasty once that chromosome is introduced into a cell of yeasty.

In contrast, “endogenous” as used herein with reference to a nucleic acid (e.g., a gene) (or a protein) and a host cell refers to any nucleic acid (or protein) that does occur in (and can be obtained from) that particular cell as it is found in nature. Moreover, a cell “endogenously expressing” a nucleic acid (or a protein) expresses that nucleic acid (or protein) as does a host cell of the same particular type as it is found in nature. Moreover, a host “endogenously producing” or that “endogenously produces” a nucleic acid, protein, or other compound produces that nucleic acid, protein, or other compound as does a host cell of the same particular type as it is found in nature.

The term “exogenous” as used herein with respect to a promoter that drives expression of a protein coding sequence means that the promoter does not drive expression of that protein coding sequence as the protein coding sequence occurs in nature. On the other hand, the term “endogenous” as used herein with respect to a promoter that drives expression of a protein coding sequence means that the promoter does drive expression of that protein coding sequence as the protein coding sequence occurs in nature.

The term “exogenous” as used herein with respect to a leader or signal sequence that is covalently bound, directly or indirectly, to a mature protein means that the leader or signal sequence is not covalently bound, directly or indirectly, to that mature protein as the corresponding immature protein occurs in nature. On the other hand, the term “endogenous” as used herein with respect to a leader or signal sequence that is covalently bound, directly or indirectly, to a mature protein means that the leader or signal sequence is covalently bound, directly or indirectly, to that mature protein as the corresponding immature protein occurs in nature. Provided herein are uses of nucleic acids encoding type I sulfatases, including iduronate sulfatase and sulfamidase, and functional fragments of them. Also featured are type I sulfatases of different origins and functional fragments of these. The use of additional nucleic acid sequences encoding proteins including FGEs of different origins (i.e., human, Streptomyces coelicolor (bacterium), Hemicentrotus pulcherrimus (sea urchin) Bos taurus (bovine), Mycobacterium tuberculosis (bacterium), Tupaia chinensis (tree shrew), Monodelphis domestica (opposum), Gallus gallus (red junglefowl), Dendroctonus ponderosa (mountain pine beetle) or Columba livia (rock dove)), various FGEs (i.e., SCO7548, Rv0712, sulfatase modifying factor 1 and C alpha formylglycine generating enzyme), trafficking proteins (i.e. PDIs, Erp44, and SUMF2), ER targeting polypeptides (e.g., those of Y. lipolytica MNS1 or WBP1), post-translational modifying enzymes (i.e., mannosidases and polypeptides that effect mannosyl phosphorylation), and functional fragments of all of these is also included. A nucleic acid encoding a polypeptide of interest (e.g., a type I sulfatase, or a functional fragment thereof), an FGE, a trafficking polypeptide, an ER targeting polypeptide (i.e. Y. lipolytica MNS1 and Y. lipolytica WBP1), a mannosidase, a polypeptide that effects mannosyl phosphorylation or a functional fragment of any of these, can be or contain, a nucleotide sequence, having at least 70% sequence identity (e.g., at least 80%, 85%, 90%, 95%, 97%, 98%, 99%, or 100% sequence identity) to the nucleotide sequences encoding the corresponding wild-type polypeptides or functional fragments. In some embodiments, nucleic acids described herein are, or can contain, a nucleotide sequence that is at least 70% (e.g., at least 75, 80, 85, 90, 93, 95, 99, or 100 percent) identical to the naturally occurring sequences and corresponding functional fragment-encoding sequences. In addition, the nucleic acids can be, or contain, nucleotide sequences, encoding the polypeptides or functional fragments of them that have at least 70% (e.g., at least 75, 80, 85, 90, 95, 99, or 100 percent) identity to the naturally occurring polypeptide amino acid sequences (e.g., those set forth in SEQ ID NO: 9, 11, 13, 15, 17, 19, 21, 43, 45, 47, 49, 51 and whose nucleic acid sequences are set forth in SEQ ID NO: 10, 12, 14, 16, 18, 20, 22, 44, 46, 48, 50, 52) or functional fragments of the naturally occurring polypeptide amino acid sequences. For example, a nucleic acid can encode a type I sulfatase having at least 90% (e.g., at least 95 or 98%) identity to the amino acid sequence set forth in SEQ ID NO: 19 (whose nucleic acid sequence is set forth in SEQ ID NO: 20) or a portion thereof.

The percent identity between a particular amino acid sequence and the amino acid sequence set forth for a protein can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.comJblas/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq 1.txt); -j is set to a file containing the second amino acid sequence to be compared (e.g., C:\seq2.txt); -p is set to blastp; -0 is set to any desired file name (e.g., C:\output.txt); and all other options are left at their default setting.

For example, the following command can be used to generate an output file containing a comparison between two amino acid sequences: C:\Bl2seq c:\seq1.txt -j c:\seq2.txt-pblastp -0 c:\output.txt. If the two compared sequences share homology, then the designated output file will present those regions of homology as aligned sequences. If the two compared sequences do not share homology, then the designated output file will not present aligned sequences. Similar procedures can be following for nucleic acid sequences except that blastn is used. Once aligned, the number of matches is determined by counting the number of positions where an identical amino acid residue is presented in both sequences. The percent identity is determined by dividing the number of matches by the length of the full-length polypeptide amino acid sequence followed by multiplying the resulting value by 100.

It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer. It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given polypeptide can be modified such that optimal expression in a particular species (e.g., bacteria or fungus) is obtained, using appropriate codon bias tables for that species. Hybridization also can be used to assess homology between two nucleic acid sequences. A nucleic acid sequence described herein, or a fragment or variant thereof, can be used as a hybridization probe according to standard hybridization techniques. The hybridization of a probe of interest to DNA or RNA from a test source is an indication of the presence of DNA or RNA corresponding to the probe in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by a wash in 0.2×SSC, 0.1% SDS at 65° C.

In addition to nucleic acids encoding the above-described wild-type and variant polypeptides and polypeptide fragments, this document also provides all the wild-type and variant polypeptides and polypeptide fragments per se.

### Enzymes and Other Polypeptides

Type I Sulfatases

This document provides the use of isolated nucleic acids encoding type I sulfatases that can hydrolyze sulfate esters as well as the type I sulfatases themselves and functional fragments thereof. Substrates of type I sulfatases include small cytosolic steroids, such as estrogen sulfate, complex cell-surface carbohydrates, such as the glycosaminoglycans, and glycolipids. Type I sulfatases function in the degradation of sulfated glycosaminoglycans and glycolipids in the lysosome, and in remodeling sulfated glycosaminoglycans in the extracellular space. Type I sulfatases include, without limitation, cerebroside-sulfatase, steroid sulfatase, arylsulfatase A, arylsulfatase B, arylsulfatase C, arylsulfatase E, iduronate 2-sulfatase, N-acetylgalactosamine-6-sulfatase, N-sulfoglucosamine sulfohydrolase, glucosamine-6-sulfatase, N-sulfoglucosamine sulfohydrolase. Sources of type I sulfatases useful for the invention include those from prokaryotes (e.g., bacteria) and eukaryotes (e.g., fungi (including yeasts), plants, insects, molluscs, and vertebrates such as mammals, fish, birds, and reptiles. A mammal can be, for example, a human or a nonhuman primate (e.g., chimpanzee, baboon, or monkey), a mouse, a rat, a rabbit, a guinea pig, a gerbil, a hamster, a horse, a type of livestock (e.g., cow, pig, sheep, or goat), a dog, a cat, or a whale. Fungi can be any of those listed herein as sources of cells for performing the methods of the document. Exemplary sources include, for example, sea urchins and green algae.

Type I sulfatases, or functional fragments thereof, undergo co- or post-translational modification for their activity in hydrolyzing sulfate esters. An active site cysteine residue is oxidized to the aldehyde-containing Ca-formylglycine residue by FGE, or funcational ragments thereof, described below. In mediating its catalytic activity, the formylglycine (FGly) residue positioned within the active site of type I sulfatases is believed to undergo hydration to a gem-diol, after which one of the hydroxyl groups acts as a catalytic nucleophile to initiate sulfate ester cleavage. The FGly residue is located within a ˜12-residue consensus sequence termed the type I sulfatase motif that defines this family of enzymes and is highly conserved throughout all domains of nature. Sources of FGE can be those listed above for sulfatases. Iduronate sulfatase has, for example, the 12 amino acid conserved sequence CAPSRVSFLTGR (SEQ ID NO: 34) (the cysteine residue that is converted to FGly is underlined (Dierks et al (1999) The EMBO Journal, 18(8), 2084-2091, the disclosure of which is incorporated herein by reference in its entirety)).

Formylglycine-Generating Enzymes

This document provides the use of isolated nucleic acids encoding formylglycine-generating enzymes (FGEs), or functional fragments thereof, that can oxidize a cysteine residue in the active site of type I sulfatase to the aldehyde-containing Ca-formylglycine residue as well as the FGEs and fragments per se. For example, FGE may be the protein product of the human gene sulfatase modifying factors 1 (SUMF1). The functional fragment of an FGE protein generally contains the active site of the FGE enzyme. The functional fragment has the ability to activate a type I sulfatase, or a functional fragment thereof. Candidate functional fragments of type I sulfatases can therefore be produced by one skilled in the art using well established methods. Their activity can be confirmed by well-established methods such as those described in the working examples disclosed here.

Sources of FGEs can be eukaryotic (e.g., bacterial) or eukaryotic (e.g., fungal (including yeast), vertebrate (e.g., mammalian), invertebrate (e.g., insect or mollusc), or plant. Thus, they can be from humans (Homo sapiens), Streptomyces coelicolor, Mycobacterim tuberculosis, Hemicentrotus pulcherrimus, Bos taurus, Mus musculus, Danio rerio, Drosophila melanogaster, Tupaia chinensis, Monodelphis domestica, Gallus gallus, Dendroctonus ponderosa, or Columba livia and the like. FGE proteins from different species are listed at this website: http://www.ebi.ac.uk/interpro/entry/IPR005532/taxonomy;jsessionid=A50B4C8B868FB85867E 9D179F3959BED.

This list is incorporated here by reference in its entirety.

Trafficking and Chaperone Proteins

Enzymes catalyzing proper protein folding are coupled to the function of protein trafficking and translocation. Certain such chaperone enzymes also aid in the transport of the proteins to different locations within a cell. By acting as a chaperone, these enzymes aid proteins to reach a correctly folded state. This document provides the use of isolated nucleic acids encoding such trafficking proteins and functional fragments thereof, as well as the proteins and functional fragments themselves. These include, for example, PDI (protein disulfide isomerase) that can (i) catalyze the formation and breakage of disulfide bonds between cysteine residues within proteins as they fold (ii) act as a chaperone protein (aid its correct folding of proteins) (iii) act as an isomerase to catalyze a reduction of mispaired thiol residues of a particular substrate (iv) catalyze the posttranslational modification disulfide exchange, and (iv) load antigenic peptides into MEW class I molecules. Genes that code for members of the PDI family include without limitation, AGR2, AGR3, CASQ1, CASQ2, DNAJC10, ERP27, ERP29, ERP44, P4HB, PDIA2, PDIA3, PDIA4, PDIA5, PDIA6, PDIALT, TMX1, TMX2, TMX3, TMX4, TXNDC5, or TXNDC12 (http://www.ncbi.nlm.nih.gov/pubmed/20796029). Also provided herein is the use of isolated nucleic acids encoding the trafficking protein ERp44 and functional fragments thereof, as well as ERp44 per se and functional fragments of it. ERp44 forms mixed disulfides with both Ero1-Lα and -Lβ (hEROs) and cargo folding intermediates. ERp44 is believed to have a role in the control of oxidative protein folding in the ER and is required to retain certain proteins in the ER.

Mannosidases

As described herein, type I sulfatases, or functional fragments thereof, containing N-glycans can be demannosylated, and type I sulfatases containing a phosphorylated N-glycan containing a terminal mannose-1-phospho-6-mannose linkage or moiety can be uncapped and demannosylated by contacting the glycoprotein with a mannosidase capable of (i) hydrolyzing a mannose-1-phospho-6-mannose linkage or moiety to mannose-6-phosphate and (ii) hydrolyzing a terminal alpha-1,2-mannose, alpha-1,3-mannose and/or alpha-1,6-mannose linkage or moiety. Non-limiting examples of such mannosidases include a Canavalia ensiformis (Jack bean) mannosidase and a Yarrowia lipolytica mannosidase (e.g., AMS1). Both the Jack bean and AMSI mannosidase are family 38 glycoside hydrolases. This document provides nucleic acids encoding such mannosidases and functional fragments of them, as well as the mannosidases per se and functional fragments thereof.

In an N-glycan bound to a type I sulfatase, or a functional fragment thereof, containing a terminal mannose-1-phospho-6-mannose moiety, there may be an additional mannose residue bound via an alpha 1,2 linkage to the mannose that is bound via its 6-position to the phosphate of the moiety. The mannose that is bound via its 6-position to the phosphate of the moiety is sometimes referred to herein as the underlying mannose residue. Upon contacting an isolated activated type I sulfatase with the purified mannosidases and/or cell lysate, the mannose-1-phospho-6-mannose linkage or moiety can be hydrolyzed to phospho-6-mannose and the terminal alpha-1,2 mannose, alpha-1,3 mannose and/or alpha-1,6 mannose linkage or moiety of such a phosphate containing glycan can be hydrolyzed to produce an uncapped and demannosylated target molecule. In some embodiments, one mannosidase is used that catalyzes both the uncapping and demannosylating steps. In some embodiments, one mannosidase is used to catalyze the uncapping step and a different mannosidase is used to catalyze the demannosylating step. The methods described in PCT/IB2011/002770 or U.S. Application Publication No. U.S. Application Publication No. US2013/0267473-A1 can be used to determine if the type I sulfatase has been uncapped and demannosylated.

This document also provides nucleic acids encoding proteins, as well as the proteins per se, with the activities of all the polypeptides described above, as well as the use of the nucleic and the proteins in the methods described herein. These polypeptides include, without limitation, any of the described type I sulfatases, FGEs, trafficking and chaperone molecules, and mannosidases. It is understood that the proteins having these activities include the full-length wild type mature (and immature as appropriate) polypeptides and functional fragments of the full-length wild type mature polypeptides, as well all of the variants of both as described herein. Examples of variants include, without limitation, those specified in terms of percent (%) identity to a reference amino acid or nucleic acid sequence, degrees of hybridization of coding nucleic acids to target nucleic acids, substitutions (e.g., conservative amino acid substitutions), additions (amino acids or nucleotides), and deletions (amino acids or nucleotides).

### Genetically Engineered Cells and Methods of Using the Same

The genetically engineered cells of the present document can contain one or more nucleic acids encoding one or more of a FGE, a type I sulfatase a trafficking protein (i.e., PDI, Erp44), one or more mannosidases, a polypeptide that effects phosphorylation of a mannose residue and functional fragments of these proteins. The nucleic acids may encode one or more copies of either FGE, type 1 sulphatase, or both. Cells suitable for in vivo production of activated type I sulfatases or for recombinant production of any of the polypeptides described herein can be of fungal origin, including yeasts such as Yarrowia lipolytica, and Arxula adeninivorans or other related species of dimorphic yeasts, Saccharomyces cerevisiae, methylotrophic yeasts (such as methylotrophic yeasts of the genus Candida, Hansenula, Ogataea, Pichia or Torulopsis) or filamentous fungi of the genus Aspergillus, Trichoderma, Neurospora, Fusarium, or Chrysosporium. Exemplary yeast species include, without limitation, Pichia anomala, Pichia bovis, Pichia canadensis, Pichia carson ii, Pichia farinose, Pichia fermentans, Pichia fluxuum, Pichia membranaefaciens, Pichia membranaefaciens, Candida valida, Candida albicans, Candida ascalaphidarum, Candida amphixiae, Candida Antarctica, Candida atlantica, Candida atmosphaerica, Candida blattae, Candida carpophila, Candida cerambycidarum, Candida chauliodes, Candida corydalis, Candida dosseyi, Candida dubliniensis, Candida ergatensis, Candidafructus, Candida glabra ta, Candida fermentati, Candida guilliermondii, Candida haemulonii, Candida insectamens, Candida insectorum, Candida intermedia, Candida jeffresii, Candida kefYr, Candida krusei, Candida lusitaniae, Candida lyxosophila, Candida maltosa, Candida membranifaciens, Candida milleri, Candida oleophila, Candida oregonensis, Candida parapsilosis, Candida quercitrusa, Candida shehatea, Candida temnochilae, Candida tenuis, Candida tropicalis, Candida tsuchiyae, Candida sinolaborantium, Candida sojae, Candida viswanathii, Candida utilis, Ogataea minuta, Pichia membranaefaciens, Pichia silvestris, Pichia membranaefaciens, Pichia chodati, Pichia membranaefaciens, Pichia menbranaefaciens, Pichia minuscule, Pichia pastoris, Pichia pseudopolymorpha, Pichia quercuum, Pichia robertsii, Pichia saitoi, Pichia silvestrisi, Pichia strasburgensis, Pichia terricola, Pichia vanriji, Pseudozyma Antarctica, Rhodosporidium toruloides, Rhodotorula glutinis, Saccharomyces bayanus, Saccharomyces bayanus, Saccharomyces momdshuricus, Saccharomyces uvarum, Saccharomyces bayanus, Saccharomyces cerevisiae, Saccharomyces bisporus, Saccharomyces chevalieri, Saccharomycesdel brueckii, Saccharomyces exiguous, Saccharomyces fermentati, Saccharomyces fragilis, Saccharomyces marxianus, Saccharomyces mellis, Saccharomyces rosei, Saccharomyces rouxii, Saccharomyces uvarum, Saccharomyces willianus, Saccharomycodes ludwigii, Saccharomycopsis capsularis, Saccharomycopsis fibuligera, Saccharomycopsis fibuligera, Endomyces hordei, Endomycopsis fobuligera. Saturnispora saitoi, Schizosaccharomyces octosporus, Schizosaccharomyces pombe, Schwanniomyces occidentalis, Torulaspora delbrueckii, Torulaspora delbrueckii, Saccharomyces dairensis, Torulaspora delbrueckii, Torulaspora fermentati, Saccharomyces fermentati, Torulaspora delbrueckii, Torulaspora rosei, Saccharomyces rosei, Torulaspora delbrueckii, Saccharomyces rosei, Torulaspora delbrueckii, Saccharomyces delbrueckii, Torulaspora delbrueckii, Saccharomyces delbrueckii, Zygosaccharomyces mongolicus, Dorulaspora globosa, Debaryomyces globosus, Torulopsis globosa, Trichosporon cutaneum, Trigonopsis variabilis, Williopsis californica, Williopsis saturnus, Zygosaccharomyces bisporus, Zygosaccharomyces bisporus, Debaryomyces disporua. Saccharomyces bisporas, Zygosaccharomyces bisporus, Saccharomyces bisporus, Zygosaccharomyces mellis, Zygosaccharomyces priorianus, Zygosaccharomyces rouxiim, Zygosaccharomyces rouxii, Zygosaccharomyces barkeri, Saccharomyces rouxii, Zygosaccharomyces rouxii, Zygosaccharomyces major, Saccharomyces rouxii, Pichia anomala, Pichia bovis, Pichia Canadensis, Pichia carson ii, Pichiafarinose, Pichiafermentans, Pichiafluxuum, Pichia membranaefaciens, Pichia pseudopolymorpha, Pichia quercuum, Pichia robertsii, Pseudozyma Antarctica, Rhodosporidium toruloides, Rhodosporidium toruloides, Rhodotorula glutinis, Saccharomyces bayanus, Saccharomyces bayanus, Saccharomyces bisporus, Saccharomyces cerevisiae, Saccharomyces chevalieri, Saccharomyces delbrueckii, Saccharomyces fermentati, Saccharomyces fragilis, Saccharomycodes ludwigii, Schizosaccharomyces pombe, Schwanniomyces occidentalis, Torulaspora delbrueckii, Torulaspora globosa, Trigonopsis variabilis, Williopsis californica, Williopsis saturnus, Zygosaccharomyces bisporus, Zygosaccharomyces mellis, or Zygosaccharomyces rouxii. Exemplary filamentous fungi include various species of Aspergillus including, but not limited to, Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowii, Aspergillus tamari, Aspergillus terre us, Aspergillus ustus, Aspergillus versicolor, Trichoderma reesei, or Neurospora crassa. Such cells, prior to the genetic engineering as specified herein, can be obtained from a variety of commercial sources and research resource facilities, such as, for example, the American Type Culture Collection (Rockville, Md.).

Genetic engineering of a cell can include, in addition to transformation with one or more nucleic acids (e.g., expression vectors) encoding one or more of an FGE, a type I sulfatase, or multiple copies thereof, a trafficking polypeptide, and one or more mannosidases (and functional fragments of these proteins or fusion proteins thereof), further genetic modifications such as: (i) deletion of an endogenous gene encoding an Outer CHain elongation (OCH) protein 1; (ii) introduction of a recombinant nucleic acid encoding a polypeptide capable of effecting mannosyl phosphorylation (e.g, a MNN4 polypeptide from Yarrowia lipolytica, S. cerevisiae, Ogataea minuta, Pichia pastoris, or C. albicans, or PNO1 polypeptide from P. pastoris) to increase phosphorylation of mannose residues; (iii) introduction or expression of an RNA molecule that interferes with the functional expression of an OCH1 protein; (iv) introduction of a recombinant nucleic acid encoding a wild-type (e.g., endogenous or exogenous) protein having a N-glycosylation activity (i.e., expressing a protein having an N-glycosylation activity); or (v) altering the promoter or enhancer elements of one or more endogenous genes encoding proteins having N-glycosylation activity to thus alter the expression of their encoded proteins. RNA molecules include, e.g., small-interfering RNA (siRNA), short hairpin RNA (shRNA), anti-sense RNA, or micro RNA (miRNA). Further genetic engineering also includes altering an endogenous gene encoding a protein having an N-glycosylation activity to produce a protein having additions (e.g., a heterologous sequence), deletions, or substitutions (e.g., mutations such as point mutations; conservative or non-conservative mutations). Mutations can be introduced specifically (e.g., by site-directed mutagenesis or homologous recombination) or can be introduced randomly (for example, cells can be chemically mutagenized as described in, e.g., Newman and Ferro-Novick (1987) J Cell Biol. 105(4):1587. It is noted the cells can contain one or more (e.g., two of more, three or more, four or more, five or more, six or more, seven or more, eight of more, nine or more, or ten or more) of these further genetic modifications. See, e.g., U.S. Pat. No. 8,026,083, the contents of which are incorporated herein by reference in its entirety, for further details on genetic engineering strategies for use in fungi such as Yarrowia lipolytica.

Genetic modifications described herein can result in one or more of (i) an increase in one or more activities in the genetically modified cell, (ii) a decrease in one or more activities in the genetically modified cell, or (iii) a change in the localization or intracellular distribution of one or more activities in the genetically modified cell. It is understood that an increase in the amount of a particular activity (e.g., promoting mannosyl phosphorylation or activating a type I sulfatase) can be due to overexpressing one or more proteins capable of promoting an activity of interest, an increase in copy number of an endogenous gene (e.g., gene duplication), or an alteration in the promoter or enhancer of an endogenous gene that stimulates an increase in expression of the protein encoded by the gene. A decrease in one or more particular activities can be due to overexpression of a mutant form (e.g., a dominant negative form), introduction or expression of one or more interfering RNA molecules that reduce the expression of one or more proteins having a particular activity, or deletion of one or more endogenous genes that encode a protein having the particular activity.

To disrupt a gene by homologous recombination, a “gene replacement” vector can be constructed in such a way to include a selectable marker gene. The selectable marker gene can be operably linked, at both 5′ and 3′ end, to portions of the gene of sufficient length to mediate homologous recombination. The selectable marker can be one of any number of genes which either complement host cell auxotrophy or provide antibiotic resistance, including URA3, LEU2 and HIS3 genes. Other suitable selectable markers include the CAT gene, which confers chloramphenicol resistance to yeast cells, or the lacZ gene, which results in blue colonies due to the expression of β-galactosidase. Linearized DNA fragments of the gene replacement vector are then introduced into the cells using methods well known in the art (see below). Integration of the linear fragments into the genome and the disruption of the gene can be determined based on the selection marker and can be verified by, for example, Southern blot analysis. A selectable marker can be removed from the genome of the host cell by, e.g., Cre-loxP systems (see below). Alternatively, a gene replacement vector can be constructed in such a way as to include a portion of the gene to be disrupted, which portion is devoid of any endogenous gene promoter sequence and encodes none or an inactive fragment of the coding sequence of the gene. An “inactive fragment” is a fragment of the gene that encodes a protein having, e.g., less than about 5% (e.g., less than about 4%, less than about 3%, less than about 2%, less than about 1%, or 0%) of the activity of the protein produced from the full-length coding sequence of the gene. Such a portion of the gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene.

Overexpressing a protein in a cell (e.g., a fungal cell) can be acheived using an expression vector. Expression vectors can be autonomous or integrative. A recombinant nucleic acid (e.g., one encoding a type I sulfatase family member, an FGE, a trafficking polypeptide, a polypeptide that effects mannosyl phosphorylation, a mannosidase, or a functional fragment of any of these) can be in introduced into the cell in the form of an expression vector such as a plasmid, phage, transposon, cosmid or virus particle. The recombinant nucleic acid can be maintained extra chromosomally or it can be integrated into the yeast cell chromosomal DNA. Expression vectors can contain selection marker genes encoding proteins required for cell viability under selected conditions (e.g., URA3, which encodes an enzyme necessary for uracil biosynthesis or TRP 1, which encodes an enzyme required for tryptophan biosynthesis) to permit detection and/or selection of those cells transformed with the desired nucleic acids (see, e.g., U.S. Pat. No. 4,704,362, the disclosure of which is incorporated herein by reference in its entirety). Expression vectors can also include an autonomous replication sequence (ARS). For example, U.S. Pat. No. 4,837,148 (the disclosure of which is incorporated herein by reference in its entirety) describes autonomous replication sequences which provide a suitable means for maintaining plasmids in Pichia pastoris.

Integrative vectors are disclosed, e.g., in U.S. Pat. No. 4,882,279, the disclosure of which is incorporated herein by reference in its entirety. Integrative vectors generally include a serially arranged sequence of at least a first insertable DNA fragment, a selectable marker gene, and a second insertable DNA fragment. The first and second insertable DNA fragments are each about 200 (e.g., about 250, about 300, about 350, about 400, about 450, about 500, or about 1000 or more) nucleotides in length and have nucleotide sequences which are homologous to portions of the genomic DNA of the species to be transformed. A nucleotide sequence containing a coding sequence of interest (e.g., a coding sequence encoding an FGE or a functional fragment of an FGE) for expression is inserted in this vector between the first and second insertable DNA fragments whether before or after the marker gene. Integrative vectors can be linearized prior to yeast transformation to facilitate the integration of the nucleotide sequence of interest into the host cell genome. An expression vector can feature a recombinant nucleic acid under the control of a yeast (e.g., Yarrowia lipolytica, Arxula adeninivorans, P. pastoris, or other suitable fungal species) promoter, which enables them to be expressed in fungal cells. As used herein, a “promoter” refers to a DNA sequence that enables a gene to be transcribed. The promoter is recognized by RNA polymerase, which then initiates transcription. Thus, a promoter contains a DNA sequence that is either bound directly by, or is involved in the recruitment, of RNA polymerase. In addition to a promoter sequence, a nucleic acid such an expression vector can include “enhancer regions,” which are one or more regions of DNA that can be bound with proteins (namely, the trans-acting factors, much like a set of transcription factors) to enhance transcription levels of genes (hence the name) in a gene-cluster. The enhancer, while typically at the 5′ end of a coding region, can also be separate from a promoter sequence and can be, e.g., within an intronic region of a gene or 3′ to the coding region of the gene.

As used herein, “operably linked” means incorporated into a genetic construct (e.g., vector) so that expression control sequences (e.g., promoters and/or enhancers) effectively control expression of a coding sequence of interest. Expression vectors can be introduced into host cells (e.g., by transformation or transfection) for expression of the encoded polypeptide, which then can be purified.

Suitable yeast promoters include, e.g., ADC1, TPI1, ADH2, hp4d, TEF1, PDX, and GallO (see, e.g., Guarente et al. (1982) Proc. Natl. Acad. Sci. USA 79(23):7410) promoters. Additional suitable promoters are described in, e.g., Zhu and Zhang (1999) Bioinformatics 15(7-8):608-611 and U.S. Pat. No. 6,265,185, the disclosures of which are incorporated herein by reference in their entirety.

A promoter can be constitutive or inducible (conditional). A constitutive promoter is understood to be a promoter whose expression is constant under the standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues. It is understood that other genetically engineered modifications can also be conditional. For example, a gene can be conditionally deleted using, e.g., a site-specific DNA recombinase such as the Cre-loxP system (see, e.g., Gossen et al. (2002) Ann. Rev. Genetics 36: 153-173 and US. Application Publication No. US2006/0014264, the disclosures of which are incorporated herein by reference in their entirety). While use of a constitutive promoter system such as TEF and quasi-constitutive hp4d do not require extraneous induction in order to induce enzyme production, inducible promoter systems may also be used and form an embodiment of this invention. Such an inducible promoter would include POX2 promoter.

A recombinant nucleic acid can be introduced into a cell described herein using a variety of methods such as the spheroplast technique or the whole-cell lithium chloride yeast transformation method. Other methods useful for transformation of plasmids or linear nucleic acid vectors into cells are described in, for example, U.S. Pat. No. 4,929,555; Hinnen et al. (1978) Proc. Nat. Acad. Sci. USA 75:1929; Ito et al. (1983) J Bacterial. 153:163; U.S. Pat. No. 4,879,231; and Sreekrishna et al. (1987) Gene 59: 115, the disclosures of each of which are incorporated herein by reference in their entirety. Electroporation and PEG 1 000 whole cell transformation procedures may also be used, as described by Cregg and Russel, Methods in Molecular Biology: Pichia Protocols, Chapter 3, Humana Press, Totowa, N.J., pp. 27-39 (1998), the disclosures of which are incorporated herein by reference in their entirety.

Transformed fungal cells can be selected for by using appropriate techniques including, but not limited to, culturing auxotrophic cells after transformation in the absence of the biochemical product required (due to the cell's auxotrophy), selection for and detection of a new phenotype, or culturing in the presence of an antibiotic which is toxic to the yeast in the absence of a resistance gene contained in the transformants. Transformants can also be selected and/or verified by integration of the expression cassette into the genome, which can be assessed by, e.g., Southern blot or PCR analysis. Prior to introducing the vectors into a target cell of interest, the vectors can be grown (e.g., amplified) in bacterial cells such as Escherichia coli (E. coli) as described above. The vector DNA can be isolated from bacterial cells by any of the methods known in the art which result in the purification of vector DNA from the bacterial milieu. The purified vector DNA can be extracted extensively with phenol, chloroform, and ether, to ensure that no E. coli proteins are present in the plasmid DNA preparation, since these proteins can be toxic to mammalian cells.

PCR can be used to amplify specific sequences from DNA as well as RNA, including sequences from total genomic DNA or total cellular RNA. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >1 00 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids also can be obtained by mutagenesis of, e.g., a naturally occurring DNA.

Expression systems that can be used for small or large scale production of polypeptides include, without limitation, microorganisms such as bacteria (e.g., E. coli) transformed with recombinant bacteriophage DNA, plasmid DNA, or cosmid DNA expression vectors containing the nucleic acid molecules, and fungal (e.g., S. cerevisiae, Yarrowia lipolytica, Arxula adeninivorans, Pichia pastoris, Hansenula polymorpha, or Aspergillus) transformed with recombinant fungal expression vectors containing the nucleic acid molecules. Useful expression systems also include insect cell systems infected with recombinant virus expression vectors (e.g., baculovirus) containing the nucleic acid molecules, and plant cell systems infected with recombinant virus expression vectors (e.g., tobacco mosaic virus) or transformed with recombinant plasmid expression vectors (e.g., Ti plasmid) containing the nucleic acid molecules. Polypeptides also can be produced using mammalian expression systems, which include cells (e.g., immortalized cell lines such as COS cells, Chinese hamster ovary cells, HeLa cells, human embryonic kidney 293 cells, and 3T3 LI cells) harboring recombinant expression constructs containing promoters derived from the genome of mammalian cells (e.g., the metallothionein promoter) or from mammalian viruses (e.g., the adenovirus late promoter and the cytomegalovirus promoter). Typically, recombinant mannosidase polypeptides are tagged with a heterologous amino acid sequence such FLAG, polyhistidine (e.g., hexahistidine), hemagluttanin (HA), glutathione-S-transferase (GST), or maltose-binding protein (MBP) to aid in purifying the protein. Other methods for purifying proteins include chromatographic techniques such as ion exchange, hydrophobic and reverse phase, size exclusion, affinity, hydrophobic charge-induction chromatography, and the like (see, e.g., Scopes, Protein Purification: Principles and Practice, third edition, Springer-Verlag, New York (1993); Burton and Harding, J Chromatogr. A 814:71-81 (1998), the disclosure of which is incorporated herein by reference in its entirety). To isolate proteins specifically from the cell culture media, the protein can be concentrated by precipitation, ultrafiltration, batch adsorption or partition in aqueous phase system. Furthermore, the isolated protein can subsequently be enriched by chromatographic techniques as mentioned as well as partition. In addition, high resolution purification of the protein can be achieved by immune-adsorption. The protein can then be subject to pyrogen removal, sterilization and formulation.

In general, for in vivo production of the activated type I sulfatases, or the functional fragments of these proteins, by fungal (e.g., Y. lipolytica) recombinant cells, the cells can be cultured in an aqueous nutrient medium comprising sources of assimilatable nitrogen and carbon, typically under submerged aerobic conditions (shaking culture, submerged culture, etc.). The aqueous medium can be maintained at a pH of 4.0-8.0 (e.g., 4.5, 5.0, 5.5, 6.0, or 7.5), using protein components in the medium, buffers incorporated into the medium or by external addition of acid or base as required. Suitable sources of carbon in the nutrient medium can include, for example, carbohydrates, lipids and organic acids such as glucose, sucrose, fructose, glycerol, starch, vegetable oils, petrochemical derived oils, succinate, formate and the like. Suitable sources of nitrogen can include, for example, yeast extract, Corn Steep Liquor, meat extract, peptone, vegetable meals, distillers solubles, dried yeast, and the like as well as inorganic nitrogen sources such as ammonium sulphate, ammonium phosphate, nitrate salts, urea, amino acids and the like.

Carbon and nitrogen sources, advantageously used in combination, need not be used in pure form because less pure materials, which contain traces of growth factors and considerable quantities of mineral nutrients, are also suitable for use. Desired mineral salts such as sodium or potassium phosphate, sodium or potassium chloride, magnesium salts, copper salts and the like can be added to the medium. An antifoam agent such as liquid paraffin or vegetable oils may be added in trace quantities as required but is not typically required.

Cultivation of recombinant cells (e.g., Y. lipolytica cells) expressing a type I sulfatase polypeptide, or functional fragment thereof, can be performed under conditions that promote optimal biomass and/or enzyme titer yields. Such conditions include, for example, batch, fed-batch or continuous culture. Further, changes to the parameters of the conditions can also promote optimal biomass and/or enzyme titer yields of the active form of type I sulfatase, or functional fragment thereof. Such conditions include, for example, glycerol concentration in the culture media, high pO₂ (see below) and the temperature selected for cultivation. For production of high amounts of biomass, submerged aerobic culture methods can be used, while smaller quantities can be cultured in shake flasks. For production in large tanks, a number of smaller inoculum tanks can be used to build the inoculum to a level high enough to minimize the lag time in the production vessel. The medium for production of the biocatalyst is generally sterilized (e.g., by autoclaving) prior to inoculation with the cells. Aeration and agitation of the culture can be achieved by mechanical means simultaneous addition of sterile air or by addition of air alone in a bubble reactor. A higher pO₂ (dissolved oxygen) can be used during cultivation in, for example, a bioreactor to promote optimal biomass. It can also be used to promote optimal active protein expression in the biomass culture. Implementation of such fermentation parameters, including a higher partial oxygen pressure and stepwise glycerol depletion, can result in an increased FGly residue conversion, indicative of active type I sulfatase. pO₂ can be 5%-40% (e.g., 10%, 15%, 20%, 25%, 30%, or 35%).

The temperature for cultivation may be from 15° C. to 32° C. (e.g., 16° C., 17° C., 18° C., 19° C., 20° C., 21° C., 22° C., 23° C., 24° C., 25° C., 26° C., 27° C., 28° C., 29° C., 30° C. or 31° C.).

Provided herein is the use of an expression system in Y. lipolytica and a customized fermentation protocol involving higher partial oxygen pressure and stepwise glycerol depletion to produce activated type I sulfatase, or a functional fragment thereof. The presence of FGly residue conversion of a formylglycine-modified peptide, indicative of an active type I sulfatase or functional fragment thereof, was determined using protocols discussed in this document. The conversion of the FGly residue, as a measure of the activation of type I sulfatase or functional fragment thereof, was in a number of instances calculated to be 100%. It is to be understood that an activation of 100% is detected at a detection limit of 0.5% and therefore includes values from 99.5% to 100%.

Active type I sulfatase polypeptides or functional fragments of them are usually secreted by the cells into the relevant culture medium and are not generally retained within the cells of the recombinant fungal cell (e.g., Yarrowia cell) and thus do not need to be extracted from the cells. However, should they be retained in the cells, they can be extracted and, if desired, purified by methods known in the art. Where the produced polypeptides are secreted from the recombinant fungal cells, they can be isolated and, as required, purified to a desired level by methods familiar to those in the art (see above).

Where any of the genetic modifications of the genetically engineered cells are inducible or conditional in the presence of an inducing cue (e.g., a chemical or physical cue), the genetically engineered cells can, optionally, be cultured in the presence of an inducing agent before, during, or subsequent to the introduction of one or more nucleic acids. For example, following introduction of the nucleic acid encoding an FGE and a type I sulfatase, and functional fragments of these proteins, the cells can be exposed to a chemical inducing agent that is capable of promoting the expression of the FGE and/or activated type I sulfatase. In such a case, relevant gene(s) can be engineered with an inducible promoter system. This document provides examples of such an inducible promoter system, in particular, POX2. Such a promoter is induced in the presence of oleic acid that is presented to the cell culture as an oleic acid feed. Where multiple inducing cues induce conditional expression of one or more proteins, the fungal cells can be contacted with multiple inducing agents. As indicated above, the activated type I sulfatase, or functional fragment thereof, is secreted into the culture medium via a mechanism provided by a coding sequence (either native to the exogenous nucleic acid or engineered into the expression vector), which directs secretion of the molecule from the cell.

The presence of an activated type I sulfatase molecule in, for example, cells (e.g., fungal cells), cell lysates or culture medium can be verified by a variety of standard protocols for detecting the presence of the activated type I sulfatase. For example, such protocols can include, but are not limited to, immunoblotting or radioimmunoprecipitation with an antibody specific for the activated type I sulfatase or for a tag (e.g., hexa-histidine) fused to the activated type I sulfatase, binding of a ligand specific for the altered, activated type I sulfatase, and/or testing for a type I sulfatase activity. Levels of activated type I sulfatase molecules can also be quantitated using a variety of protocols including nano-ultra high pressure liquid chromatography together with high resolution tandem mass spectrometry. Provided herein is the use of such a protocol to measure the presence of formylglycine modified peptide (FGly residue conversion), which is indicative of an activated type I sulfatase.

The proportion of type I sulfatase molecules in a preparation produced by the methods of the present document in which the cysteine to FGly conversion has occurred is greater than 10% (e.g., greater than: 20%; 30%; 40%; 50%; 60%; 70%; 80%; 85%; 90%; 92%; 95%; 97%; 98%; 99;%; or is even 100%).

In some embodiments, following isolation, the activated type I sulfatase, or functional fragment thereof, can be attached to a heterologous moiety, e.g., using enzymatic or chemical means. A “heterologous moiety” refers to any constituent that is joined (e.g., covalently or non-covalently) to the activated type I sulfatase, or functional fragment thereof, which constituent is different from a constituent originally linked to the type I sulfatase molecule, or functional fragment thereof. Heterologous moieties include, e.g., polymers, carriers, adjuvants, immunotoxins, or detectable (e.g., fluorescent, luminescent, or radioactive) moieties. In some embodiments, an additional N-glycan can be added to the altered target molecule.

Methods for detecting glycosylation of a molecule include DNA sequencer assisted (DSA), fluorophore-assisted carbohydrate electrophoresis (FACE) or surface enhanced laser desorption/ionization time-of-flight mass spectrometry (SELDI-TOF MS). For example, an analysis can utilize DSA-FACE in which, for example, glycoproteins are denatured followed by immobilization on, e.g., a membrane. The glycoproteins can then be reduced with a suitable reducing agent such as dithiothreitol (DTT) or β-mercaptoethanol. The sulfhydryl groups of the proteins can be carboxylated using an acid such as iodoacetic acid. Next, the N-glycans can be released from the protein using an enzyme such as N-glycosidase F. N-glycans, optionally, can be reconstituted and derivatized by reductive amination. The derivatized N-glycans can then be concentrated. Instrumentation suitable for N-glycan analysis includes, e.g., the ABI PRISM® 377 DNA sequencer (Applied Biosystems). Data analysis can be performed using, e.g., GENESCAN® 3.1 software (Applied Biosystems). Isolated mannoproteins can be further treated with one or more enzymes such as calf intestine phosphatase to confirm their N-glycan status. Additional methods of N-glycan analysis include, e.g., mass spectrometry (e.g., MALDI-TOF-MS), high-pressure liquid chromatography (HPLC) on normal phase, reversed phase and ion exchange chromatography (e.g., with pulsed amperometric detection when glycans are not labeled and with UV absorbance or fluorescence if glycans are appropriately labeled). See also Callewaert et al. (2001) Glycobiology 11(4):275-281 and Freire et al. (2006) Bioconjug. Chem. 17(2):559-564.

### Cultures of Engineered Cells

This document also provides a substantially pure culture of any of the genetically engineered cells described herein. As used herein, a “substantially pure culture” of a genetically engineered cell is a culture of that cell in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the genetically engineered cell, e.g., bacterial, fungal (including yeast), mycoplasmal, or protozoan cells. The term “about” in this context means that the relevant percentage can be 15% percent of the specified percentage above or below the specified percentage. Thus, for example, about 20% can be 17% to 23%. Such a culture of genetically engineered cells includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

The genetically engineered cells described herein can be stored, for example, as frozen cell suspensions, e.g., in buffer containing a cryoprotectant such as glycerol or sucrose, as lyophilized cells. Alternatively, they can be stored, for example, as dried cell preparations obtained, e.g., by fluidized bed drying or spray drying, or any other suitable drying method.

Additional descriptions of glycosylation engineering, mannosidases, uncapping of mannose-1-phosphate-6-mannose linkages and demannosylation of phosphorylated N-glycans and additional methods of facilitating mammalian cellular uptake of glycoproteins can be found in multiple references. These references include PCT application PCT/I132011/002770, U.S. Pat. No. 8,026,083, U.S. Patent application 61/611,485, U.S. patent application Ser. No. 13/499,061, U.S. patent application Ser. No. 13/510,527, and PCT application PCT/IB2011/002780, the disclosures of all of which are incorporated herein by reference in their entirety.

Disorders Treatable with an Activated Type I Sulfatase and Functional Fragments Thereof

Activated type I sulfatases and functional fragments thereof, optionally with any N-glycans uncapped and demannosylated as described herein, can be used to treat a variety of metabolic disorders. A metabolic disorder is one that affects the production of energy within individual human (or animal) cells. Most metabolic disorders are genetic, though some can be “acquired” as a result of diet, toxins, infections, etc. Genetic metabolic disorders are also known as inborn errors of metabolism. In general, the genetic metabolic disorders are caused by genetic defects that result in missing or improperly constructed enzymes (e.g., type I sulfatases or FGEs, or functional fragments of these proteins,) necessary for some step in the metabolic process of the cell. The largest classes of metabolic disorders are disorders of carbohydrate metabolism, disorders of amino acid metabolism, disorders of organic acid metabolism (organic acidurias), disorders of fatty acid oxidation and mitochondrial metabolism, disorders of porphyrin metabolism, disorders of purine or pyrimidine metabolism, disorders of steroid metabolism disorders of mitochondrial function, disorders of peroxisomal function, and lysosomal storage disorders (LSDs).

Examples of disorders that can be treated through the administration of one or more activated type I sulfatases molecules, or functional fragment thereof, optionally uncapped and demannosylated as described herein, (or pharmaceutical compositions of the same) can include metachromatic leukodystrophy, Hunter disease, Sanfilippo disease A & D, Morquio disease A, Maroteaux-Lamy disease, X-linked ichthyosis, Chondroplasia Punctata 1, and MSD.

Symptoms of disorders treatable with activated type I sulfatase, or a functional fragment thereof, are numerous and diverse and can include one or more of e.g., anemia, fatigue, bruising easily, low blood platelets, liver enlargement, spleen enlargement, skeletal weakening, lung impairment, infections (e.g., chest infections or pneumonias), kidney impairment, progressive brain damage, seizures, extra thick meconium, coughing, wheezing, excess saliva or mucous production, shortness of breath, abdominal pain, occluded bowel or gut, fertility problems, polyps in the nose, clubbing of the finger/toe nails and skin, pain in the hands or feet, angiokeratoma, decreased perspiration, corneal and lenticular opacities, cataracts, mitral valve prolapse and/or regurgitation, cardiomegaly, temperature intolerance, difficulty walking, difficulty swallowing, progressive vision loss, progressive hearing loss, hypotonia, macroglossia, areflexia, lower back pain, sleep apnea, orthopnea, somnolence, lordosis, or scoliosis. It is understood that due to the diverse nature of the defective or absent proteins and the resulting disease phenotypes (e.g., symptomatic presentation of a metabolic disorder), a given disorder will generally present only symptoms characteristic to that particular disorder.

In addition to the administration of one or more of the active type I sulfatases, or functional fragments thereof, described herein, an appropriate disorder can also be treated by proper nutrition and vitamins (e.g., cofactor therapy), physical therapy, and pain medications. Depending on the specific nature of a given disorder, a patient can present these symptoms at any age. In many cases, symptoms can present in childhood or in early adulthood.

As used herein, a subject “at risk of developing a disorder treatable with an activated type I sulfatase, or a functional fragment thereof,” is a subject that has a predisposition to develop a disorder, i.e., a genetic predisposition to develop such a disorder as a result of a mutation in one or more genes encoding any of the type I sulfatases and FGEs disclosed herein.

A subject “suspected of having a disorder treatable with an activated type I sulfatase, or a functional fragment thereof,” is one having one or more symptoms of such a disorder.

Clearly, neither subjects “at risk of developing a disorder treatable with an activated type I sulfatase, or a functional fragment thereof,” nor those “suspected of having a disorder treatable with an activated type I sulfatase, or a functional fragment thereof” are all the subjects within a species of interest.

### Pharmaceutical Compositions and Methods of Treatment

One or more activated type I sulfatases, or functional fragments thereof, made by one or more of the methods disclosed herein can be incorporated into a pharmaceutical composition containing a therapeutically effective amount of the one or more activated type I sulfatases, or functional fragments thereof, and one or more adjuvants, excipients, carriers, and/or diluents and used in therapeutic regimens. Acceptable diluents, carriers and excipients typically do not adversely affect a recipient's homeostasis (e.g., electrolyte balance). Acceptable carriers include biocompatible, inert or bioabsorbable salts, buffering agents, oligo- or polysaccharides, polymers, viscosity improving agents, preservatives and the like. One exemplary carrier is physiologic saline (0.15 M NaCl, pH 7.0 to 7.4). Another exemplary carrier is 50 mM sodium phosphate, 100 mM sodium chloride. Further details on techniques for formulation and administration of pharmaceutical compositions can be found in, e.g., Remington's Pharmaceutical Sciences (Maack Publishing Co., Easton, Pa.). Supplementary active compounds can also be incorporated into the compositions.

Administration of a pharmaceutical composition as disclosed herein can be systemic or local. Pharmaceutical compositions can be formulated such that they are suitable for parenteral and/or non-parenteral administration. Specific administration modalities include subcutaneous, intravenous, intramuscular, intraperitoneal, transdermal, intrathecal, oral, rectal, buccal, topical, nasal, ophthalmic, intra-articular, intra-arterial, sub-arachnoid, bronchial, lymphatic, vaginal, and intra-uterine administration.

Administration can be by periodic injections of a bolus of the pharmaceutical composition or can be uninterrupted or continuous by intravenous or intraperitoneal administration from a reservoir which is external (e.g., an IV bag) or internal (e.g., a bio-erodable implant, a bio-artificial organ, or a colony of implanted altered N-glycosylation molecule production cells). See, e.g., U.S. Pat. Nos. 4,407,957, 5,798,113, and 5,800,828. Administration of a pharmaceutical composition can be achieved using suitable delivery means such as: a pump (see, e.g., Annals of Pharmacotherapy, 27:912 (1993); Cancer, 41: 1270 (1993); Cancer Research, 44: 1698 (1984); microencapsulation (see, e.g., U.S. Pat. Nos. 4,352,883; 4,353,888; and 5,084,350); continuous release polymer implants (see, e.g., Sabel, U.S. Pat. No. 4,883,666); macro encapsulation (see, e.g., U.S. Pat. Nos. 5,284,761, 5,158,881, 4,976,859 and 4,968,733 and published PCT patent applications WO92119195, WO 95/05452); injection, either subcutaneously, intravenously, intra-arterially, intramuscularly, or to other suitable site; or oral administration, in capsule, liquid, tablet, pill, or prolonged release formulation. Examples of parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aerosolizer, electroporation, and trans dermal patch.

Formulations suitable for parenteral administration conveniently contain a sterile aqueous preparation of the activated type I sulfatase, or the functional fragment thereof, which preferably is isotonic with the blood of the recipient (e.g., physiological saline solution). Formulations can be presented in unit-dose or multi-dose form.

Formulations suitable for oral administration can be presented as discrete units such as capsules, cachets, tablets, or lozenges, each containing a predetermined amount of the activated type I sulfatase; or a suspension in an aqueous liquor or anon-aqueous liquid, such as a syrup, an elixir, an emulsion, or a draught.

An activated type I sulfatase, or functional fragment thereof, made by a method disclosed herein and suitable for topical administration can be administered to a mammal (e.g., a human patient) as, e.g., a cream, a spray, a foam, a gel, an ointment, a salve, or a dry rub. A dry rub can be rehydrated at the site of administration. The activated type I sulfatase molecules, or functional fragments thereof, can also be infused directly into (e.g., soaked into and dried) a bandage, gauze, or patch, which can then be applied topically. The activated type I sulfatase, or functional fragment thereof, can also be maintained in a semi-liquid, gelled, or fully-liquid state in a bandage, gauze, or patch for topical administration (see, e.g., U.S. Pat. No. 4,307,717).

Therapeutically effective amounts of a pharmaceutical composition can be administered to a subject in need thereof in a dosage regimen ascertainable by one of skill in the art. For example, a composition can be administered to the subject, e.g., systemically at a dosage of activated type I sulfatase from 0.01 μg/kg to 10,000 μg/kg body weight of the subject, per dose. In another example, the dosage is from 1 μg/kg to 100 μg/kg body weight of the subject, per dose. In another example, the dosage is from 1 μg/kg to 30 μg/kg body weight of the subject, per dose, e.g., from 3 μg/kg to 10 μg/kg body weight of the subject, per dose.

In order to optimize therapeutic efficacy, an activated type I sulfatase, or functional fragment thereof, can be first administered at different dosing regimens. The unit dose and regimen depend on factors that include, e.g., the species of mammal, its immune status, the body weight of the mammal. Typically, levels of a such a molecule in a tissue can be monitored using appropriate screening assays as part of a clinical testing procedure, e.g., to determine the efficacy of a given treatment regimen.

The frequency of dosing for an activated type I sulfatase, or functional fragment thereof, is within the skills and clinical judgment of medical practitioners (e.g., doctors or nurses). Typically, the administration regime is established by clinical trials which may establish optimal administration parameters. However, the practitioner may vary such administration regimes according to the subject's age, health, weight, sex and medical status. The frequency of dosing can be varied depending on whether the treatment is prophylactic or therapeutic.

Toxicity and therapeutic efficacy of activated type I sulfatases (or functional fragments thereof) or pharmaceutical compositions thereof can be determined by known pharmaceutical procedures in, for example, cell cultures or experimental animals. These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosages of an activated type I sulfatase, or functional fragment thereof, for use in appropriate subjects (e.g., human patients). The dosage of activated type I sulfatase, or functional fragment thereof, in such pharmaceutical compositions lies generally within a range of circulating concentrations that include the ED₅₀ with little or no toxicity. The dosage may vary within this range depending upon the dosage form employed and the route of administration utilized. For a pharmaceutical composition used as described herein the therapeutically effective dose can be estimated initially from cell culture assays. A dose can be formulated in animal models to achieve a circulating plasma concentration range that includes the IC₅₀ (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

As defined herein, a “therapeutically effective amount” of an activated type I sulfatase, or functional fragment thereof, is an amount of activated type I sulfatase, or functional fragment thereof, that is capable of producing a medically desirable result (e.g., amelioration of one or more symptoms of the relevant disorder) in a treated subject. A therapeutically effective amount (i.e., an effective dosage) can includes milligram or microgram amounts of the compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

The subject can be any mammal, e.g., a human (e.g., a human patient) or a nonhuman primate (e.g., chimpanzee, baboon, or monkey), a mouse, a rat, a rabbit, a guinea pig, a gerbil, a hamster, a horse, a type of livestock (e.g., cow, pig, sheep, or goat), a dog, a cat, or a whale.

An activated type I sulfatase (or functional fragment thereof) or pharmaceutical composition thereof described herein can be administered to a subject as a combination therapy with another treatment, e.g., a treatment for a metabolic disorder (e.g., a lysosomal storage disorder). For example, the combination therapy can include administering to the subject (e.g., a human patient) one or more additional agents that provide a therapeutic benefit to the subject who has, or is at risk of developing, (or suspected of having) the relevant disorder (e.g., a disorder due to the absence of an active type I sulfatase). Thus, the activated type I sulfatase (or functional fragment thereof) or pharmaceutical composition thereof and the one or more additional agents can be administered at the same time. Alternatively, the activated type I sulfatase, or functional fragment thereof, can be administered first and the one or more additional agents administered second, or vice versa.

It will be appreciated that in instances where a previous therapy is particularly toxic (e.g., a treatment with significant side-effect profiles), administration of an activated type I sulfatase, or functional fragment thereof, described herein can be used to offset and/or lessen the amount of the previously therapy to a level sufficient to give the same or improved therapeutic benefit, but without the toxicity.

Any of the pharmaceutical compositions described herein can be included in a container, pack, or dispenser together with instructions for administration.

### EXAMPLES

The methods and materials of the disclosure are further described in the following examples, which do not limit the scope of the invention described in the claims.

### Example 1

### Expression of Human Iduronate-Sulfatase (IDS) in Yarrowia lipolytica

The 525 amino acid human IDS precursor (SEQ ID NO 19; corresponding encoding nucleic acid sequence set forth in SEQ ID NO: 20) was synthesized and codon-optimized for expression in Y. lipolytica. The synthetic open reading frame (ORF) of human IDS (hIDS) was fused in frame to the N-terminal region of the Y. lipolytica signal sequence Lip2pre of the extracellular lipase gene. This coding sequence was followed by two XXX-Ala cleavage sites and flanked by BamHI and AvrII restriction sites for cloning into the expression vector in which the coding sequence was under the control of the inducible POX2 promoter.

The recombinant Y. lipolytica strain carrying one stably integrated copy of POX2 driven hIDS was generated according to established protocols. The Y. lypolytica strain used in all the following examples contained the following modifications: Δoch1, URA3::POX2-MNN4; OCH1::Hp4d-MNN4; POX2-Lip2pre-hIDS::zeta. The labeling reference of the engineered strain nomenclature is as follows: deletion/insertion of a gene, locus in which the expression cassette is integrated::identification of the expression cassette integrated. In order to select high recombinant human IDS (rhIDS) expressing clones, several clones were selected at random and were grown in 24-well plates under oleic acid inducing conditions according to a standard protocol. In each case, the culture supernatant was collected 72 hours post-induction and subsequently screened using SDS-PAGE gel and standard Western blot.

### Example 2

### Co-Expression of Recombinant FGE (rFGE) in a Yarrowia lipolytica Stains Expressing rhIDS

To achieve high levels of cysteine conversion to FGly in type I sulfatases produced in Y. lipolytica strains co-expressing type I sulfatase with FGE proteins were derived. The FGE proteins were from different origins, including prokaryotic origin (Mycobacterium tuberculosis FGE (MtFGE) and Streptomyces coelicolor FGE (ScFGE)) and eukaryotic origin (Human FGE (hFGE), Bos taurus FGE (BtFGE), and Hemicentrotus pulcherrimus (HpFGE)). The FGEs and their GenBank accession numbers are shown in Table 1.

TABLE 1
FGEs from Different Sources

| FGE selected for co-expression | Accession |
|---|---|
| protein SCO7548 [Streptomyces coelicolor A3(2)] | NP_631591.1 |
| hypothetical protein Rv0712 | NP_215226.1 |
| [Mycobacterium tuberculosis H37Rv] | |
| sulfatase modifying factor 1 | BAJ83907 |
| [Hemicentrotus pulcherrimus] | |
| C-alpha-formyglycine-generating enzyme | AA034683 |
| [Homo sapiens] | |
| Sulfatase-modifying factor 1 precursor | NP_001069544 |
| [Bos taurus] | |

Genome mining with the human FGE sequence as a template resulted in the identification Genome mining with the human FGE sequence as a template resulted in the identification of putative FGE orthologs in M. tuberculosis and Streptomyces coelicolor (Carlson et al (2008), The Journal of Biological Chemistry, 283, 20117-20125). Co-expression with M. tuberculosis FGE was used to modify proteins at specific sites using an E. coli expression system; this resulted in a FGly formation with an efficiency of 85% (Rabuka et al (2012), Nature Protocols, 7, 1052-1067). Hemicentrotus pulcherrimus FGE (HpSumf1 gene product) has been shown to be involved in the activation of type I sulfatases responsible for the regulation of skeletogenesis during sea urchin development (Sakuma et al (2011), Development Genes and Evolution, 221, 157-166). Two cysteine residues, Cys₃₃₆ and Cys₃₄₁ (residue numbering based on sequence of mature hFGE) are localized in the substrate binding groove and are essential for catalytic activity of human Sumf1.

HpSumf1 also has a conserved potential N-glycosylation site at the corresponding position to human Sumf1 and a long N-terminal extension. Moreover, H. pulcherrimus FGE has been shown to be able to activate mammalian ArsA when overexpressed in HEK293T cells (Sakuma et al (2011), Development Genes and Evolution, 221, 157-166).

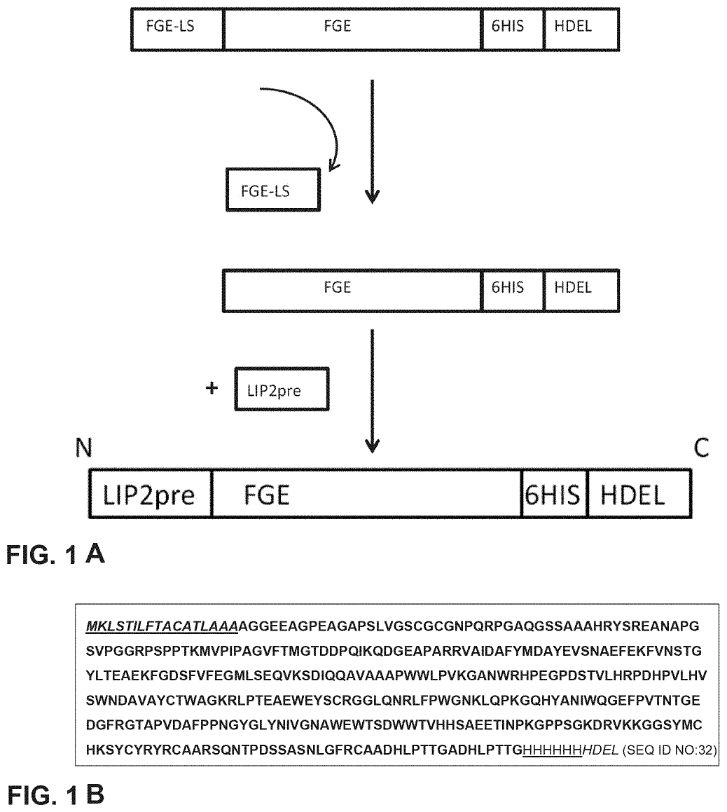

To target the different FGE enzymes to the ER, the Y. lipolytica LIP2 pre leader sequence (SEQ ID NO: 5; corresponding nucleic acid sequence set forth in SEQ ID NO: 6) was fused to the N-terminus of the mature sequences of the FGEs. The mature sequence of FGE does not contain the hFGE leader sequence (signal peptide) (SEQ ID NO: 23) which effects secretory pathway targeting. To target all the FGEs to the ER, a C-terminal HDEL tetrapeptide (SEQ ID NO: 1; corresponding nucleic acid sequences set forth in SEQ ID NOS: 2) was added as is depicted in FIG. 1 to FGEs. Upstream of the HDEL sequence a hexahistidine (6HIS) tag (SEQ ID NO: 7; corresponding nucleic acid sequence set forth in SEQ ID NOS: 8) was included to allow immunological detection. A graphical illustration of the method of construction of the FGE constructs is provided in FIG. 1A.

The amino acid sequences of the rFGE proteins that were co-expressed in a Y. lipolytica strain expressing human type I sulfatase are those of SEQ ID NOs: 9, 11, 13, 15, 17 (corresponding nucleic acid sequences set forth in SEQ ID NOS: 10, 12, 14, 16, 18, respectively).

All FGE coding sequences were synthesized and codon-optimized for expression within Y. lipolytica and were flanked by BamHI and AvrII restriction sites for cloning of the segment into an expression vector under the control of the inducible POX2 or Hp4d promoter. A summary of the co-expression strains is shown in Table 2. Each strain carried one copy of the rhIDS coding sequence co-expressed with two copies of either human (h), Bos taurus (Bt), Streptomyces coelicolor (Sc), Hemicentrotus pulcherrimus (Hp) or Mycobacterium tuberculosis (Mt) rFGE coding sequence. In each strain, one rFGE was expressed under the hp4d promoter and the other was expressed under the POX2 promoter.

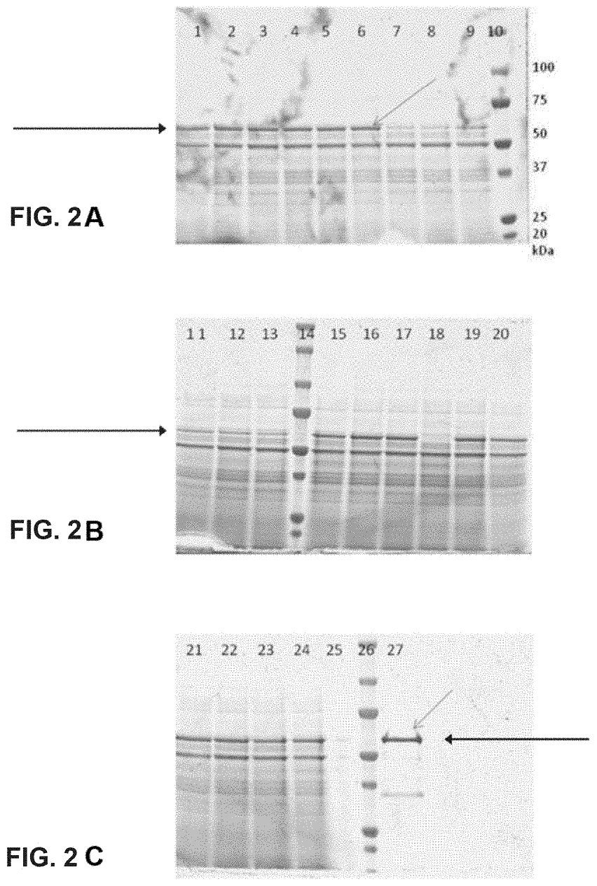

In order to select high rhIDS expressing clones, several clones were selected at random and were grown in 24-well plates under oleic acid inducing-conditions according to a standard protocol. In each case, the culture supernatant was collected 72 hours post-induction and screened by SDS-PAGE. FIG. 2 shows SDS-PAGE detection of human rIDS (SEQ ID NO 22; corresponding amino acid sequence set forth in SEQ ID NO: 21) produced in Y. lipolytica at 28° C., 24 deep well plate induction conditions. Samples were treated with Peptide-N-Glycosidase F (PNGaseF) to remove N-glycans. Lanes 1 to 6 are T146 (OXYY1828; BtFGE) clones A to F, respectively (FIG. 2A); lanes 7 to 13 are T147 (OXYY1831; ScFGE) clones A to F, respectively (FIGS. 2 A AND 2B); lanes 15 to 20 are T148 (OXYY1801; HpFGE) clones A to F, respectively (FIG. 2B); lanes 21 to 24 are T126 (OXYY1827; hFGE) clones A to D, respectively (FIG. 2C); lane 25 is empty (FIG. 2C); lane 27 contains commercial ELAPRASE® (FIG. 2C); lanes 10, 14, 26 contain protein molecular weight markers (BioRad; Hercules, Calif.) (FIG. 2A-C). The molecular weights of the markers shown in FIGS. 2B and 2C can be deduced from the labelled ones in FIG. 2A in which the same combination of molecular weight markers were used.

TABLE 2
Y. lipolytica Strains Co-Expressing Human
rIDS and rFGE from Different Sources

| rhIDS | rhFGE | |
|---|---|---|
| strains | coexpression | Strain genotype |
| OXYY1827 | HumanFGE | MATA, leu2-958, ura3-302, xpe2-322, ade2-844, |
| (T126) | | ΔSc suc2, Δoch1, URA3 ::POX2-MNN4, OCH1 |
| | | ::Hp4d-MNN4, POX2-Lip2pre-hIDS:URA3Ex::zeta, |
| | | Hp4d-Lip2pre-hFGE:Leu2Ex::zeta, POX2-Lip2pre- |
| | | hFGE:Ade2Ex::zeta |
| OXYY1828 | BtFGE | MATA, leu2-958, ura3-302, xpe2-322, ade2-844, |
| (T146) | | ΔSc suc2, Δoch1, URA3 ::POX2-MNN4, OCH1 |
| | | ::Hp4d-MNN4, POX2-Lip2pre-hIDS:URA3Ex::zeta, |
| | | Hp4d-Lip2pre-BtFGE:Leu2Ex::zeta, POX2- |
| | | Lip2pre-BtFGE:Ade2Ex::zeta |
| OXYY1831 | ScFGE | MATA, leu2-958, ura3-302, xpe2-322, ade2-844, |
| (T147) | | ΔSc suc2, Δoch1, URA3 ::POX2-MNN4, OCH1 |
| | | ::Hp4d-MNN4, POX2-Lip2pre-hIDS:URA3Ex::zeta, |
| | | Hp4d-Lip2pre-ScFGE:Leu2Ex::zeta, POX2- |
| | | Lip2pre-ScFGE:Ade2Ex::zeta |
| OXYY1801 | HpFGE | MATA, leu2-958, ura3-302, xpe2-322, ade2-844, |
| (T148) | | ΔSc suc2, Δoch1, URA3 ::POX2-MNN4, OCH1 |
| | | ::Hp4d-MNN4, POX2-Lip2pre-hIDS:URA3Ex::zeta, |
| | | Hp4d-Lip2pre-HpFGE:Leu2Ex::zeta, POX2- |
| | | Lip2pre-HpFGE:Ade2Ex::zeta |
| OXYY182 | MtFGE | MATA, leu2-958, ura3-302, xpe2-322, ade2-844, |
| (T153) | | ΔSc suc2, Δoch1, URA3 ::POX2-MNN4, OCH1 |
| | | ::Hp4d-MNN4, POX2-Lip2pre-hIDS:URA3Ex::zeta, |
| | | Hp4d-Lip2pre-MtFGE:Leu2Ex::zeta, POX2- |
| | | Lip2pre-MtFGE:Ade2Ex::zeta |

Recombinant hIDS was expressed in the presence of FGE from different sources in Y. lipolytica strains. Co-expression with FGE from Bos Taurus and Hemicentrotus pulcherrimus resulted in expression of IDS. Co-expression with FGE from Streptomyces coelicolor, however resulted in suppressed levels of IDS expression relative to that of the other strains.

### Example 3

### Detection of Intracellular FGE Expression

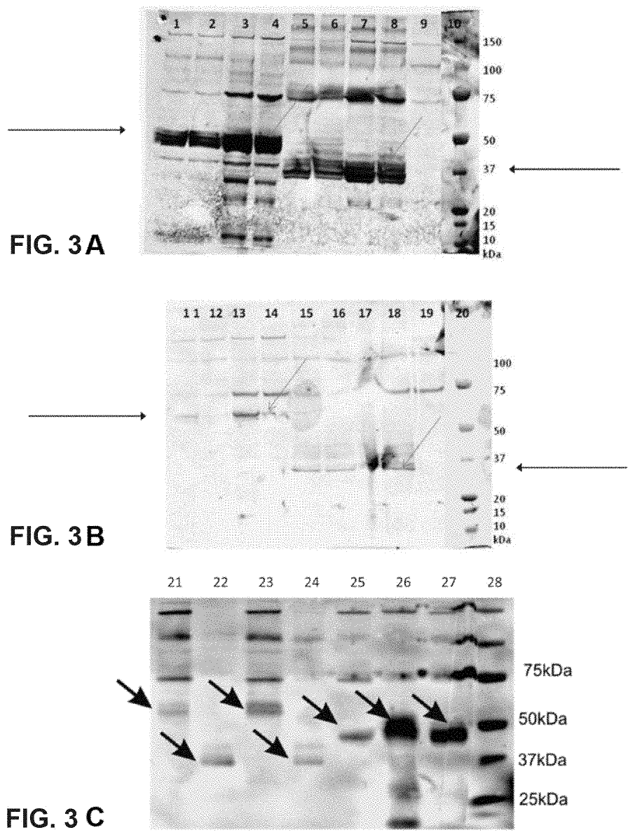

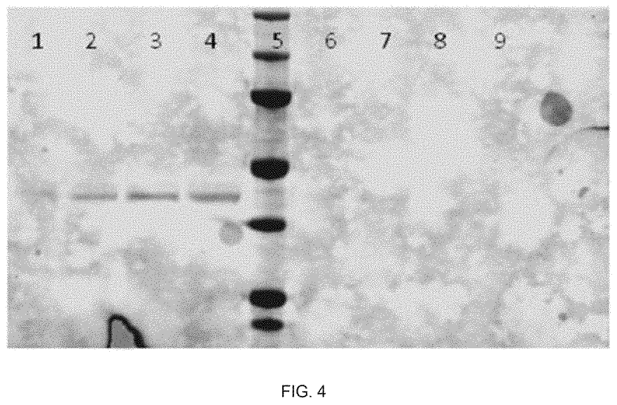

Y. lipolytica cells were harvested 96 hours following the oleic acid induction phase. Yeast cell lysates containing 6HIS-tagged rFGE were prepared according to standard procedures. The expression level of each of the different rFGE proteins was evaluated utilizing Western blot analysis with anti-HIS antibody (Geneart THEtm). The results are shown in FIG. 3 and FIG. 4. The expected molecular weights of the expressed proteins are as follows: 40.3 kDa for Bos taurus FGE, 36.7 kDa for Streptomyces coelicolor, 47.5 kDa for H. pulcherrimus; 36.1 kDa for M. tuberculosis; and 40.6 kDa for Homo sapiens.

FIG. 3 presents Western blot detection of rFGE utilizing an anti-His6 antibody (Geneart THEtm; 1:5000). Recombinant FGE is indicated by arrows. Lanes 1 to 4 are T146 (OXYY1828; BtFGE) clones A and B grown at 28° C. (lanes 1 and 2) and 20° C. (lanes 3 and 4), respectively (FIG. 3A); lanes 5-8 are T147 (OXYY1831; ScFGE) clones A and B grown at 28° C. (lanes 5 and 6) and 20° C. (lanes 7 and 8), respectively (FIG. 3B); lanes 9 and 19 are T126 (OXYY1827; hFGE) grown at 28° C. and 20° C., respectively (FIGS. 3A AND 3B); lanes 11-14 are T148 (OXYY1801; HpFGE) clones A and B grown at 28° C. (lanes 11 and 12) and 20° C. (lanes 13 and 14), respectively (FIG. 3B); lanes 15-18 are T153 (OXYY1802; MtFGE) clones A and B grown at 28° C. (lanes 15 and 16) and 20° C. (lanes 17 and 18), respectively (FIG. 3B). Lane 21 is a clone of T148 (OXYY1801; HpFGE) grown at 28° C. (FIG. 3C); lane 22 is a clone of T153 (OXYY1802; MtFGE) grown at 28° C. (FIG. 3C); lane 23 is a clone of T148 (OXYY1801; HpFGE) grown at 20° C. (FIG. 3C); lane 24 is a clone of T153 (OXYY1802; MtFGE) grown at 20° C. (FIG. 3C); lane 25 is a clone of T161 (OXYY1798; BtFGE) grown at 28° C. (FIG. 3C); Lane 26 is a clone of T156 (OXYY1803; BtFGE and hPDI) grown at 28° C. (FIG. 3C); Lane 27 is a clone of T146 (OXYY1828; BtFGE) grown at 28° C. (FIG. 3C). Lanes 10, 20, and 28 contain protein molecular weight markers (BioRad; Hercules, Calif.) (FIG. 3A-C). T126 (OXYY1827) expresses human FGE without a hexahistidine (His6) tag and is the negative control for His6 tagged detection.

Human recombinant FGE detection by Western blot utilizing commercial anti-human SUMF1 polyclonal goat antibody is shown in FIG. 4. Lanes 1-4 are T126 (OXYY1827; hFGE) clones A and B grown at 28° C. and 20° C. (reducing conditions), respectively; and lanes 6-9 are OXYY1827 clones A and B grown at 28° C. and 20° C. (non-reducing conditions), respectively. Lane 5 contains protein molecular weight markers (BioRad; Hercules, Calif.).

Different levels of expression were observed for FGE from different sources in Y. lipolytica strains. Bos taurus FGE presented the strongest expression of the differently-sourced FGEs analyzed. Hemicentrotus pulcherrimus and Mycobacterium tuberculosis FGE expressed similar levels of FGE but at levels less than those of Bos taurus and Streptomyces coelicolor derived FGE. FGE from Streptomyces coelicolor expressed at levels less than those of Bos taurus but more than those of Hemicentrotus pulcherrimus and Mycobacterium tuberculosis.

### Example 4

### Fermentation of a Yarrowia lipolytica Strain Expressing rhIDS and Co-Expressing FGE

For the production of rhIDS in Yarrowia lipolytica, a culture was established via the following two-phase method comprising:
1) Growth of the culture on glucose for biomass formation: Strains were grown under standard conditions of pH 6.8, 1 vvm air, 28° C., DO=20% with stirring cascade in 500 mL MSI+5 g/L glycerol.
2) Feed phase I for biomass generation was started following glycerol depletion (DO-spike); 60% glycerol+MSA linear feed (0.27*t+1.08)/1.12 for 24 hours.
3) Feed phase II began following feed phase II (4 hours): 60% glycerol+MSA exponential feed 0.4011*exp(0.007*t)/1.12+20% OA exponential feed 0.8022*exp (0.007*t)/0.978 until the end of the fermentation process.

TABLE 3
Overview of Fermentation Protocol of Yarrowia lipolytica

| Culture medium | Feed phase I | Feed phase II |
|---|---|---|
| 500 mL MSI + | (0.27xt(h) + | 20% Oleic Acid: |
| 5 g/L glycerol | 1.08)/1.12 | 0.72exp(0.007xt(h)/0.978 + |
| | 60% glycerol + | 60% Glycerol + MSA: |
| | MSA → 24 h | 0.39exp(0.007xt(h)/1.12 |

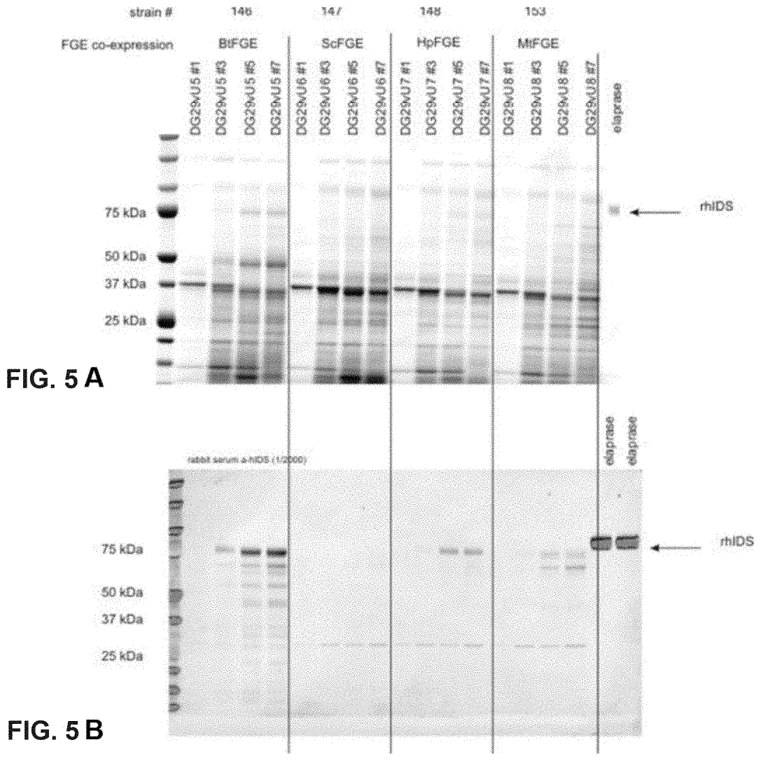

Table 3 presents the fermentation process for the production the bacteria in the bioreactor. The resulting 50 mL culture was centrifuged for 40 minutes at 7000 rpm. The supernatant was retrieved and stored at −20° C. Ten (10) μl aliquots of the supernatant were analyzed on SDS-PAGE and Western blot as shown in FIG. 5.

FIG. 5 shows expression analysis of IDS from strains co-expressing rIDS and rFGE grown under fed-batch fermentation by SDS-PAGE (FIG. 5A) and Western blot (FIG. 5B). The supernatant of each culture was analyzed for four FGE strains at four timepoints (11.1 hours, 58.8 hours, 131.6 hours, and 154.9 hours from the start of induction): T146 (OXYY1828; BtFGE co-expression), T147 (OXYY1831; ScFGE co-expression), T148 (OXYY1801; HpFGE co-expression), T153 (OXYY1802; MtFGE co-expression). rhIDS was detected with a rabbit anti-hIDS antiserum. Y. lipolytica produced rhIDS is visible at an approximate MW of 76 kDa. The four timepoints refer to the samplings in feed phase II. Each timepoint had a 24 hour space in between.

Strains of Y. lipolytica co-expressing rhIDS and recombinant FGE (rFGE) were successfully cultivated in the bioreactor. Expression levels of rhIDS however were dependent on the source of the FGE. When co-expressed with FGE derived from Bos taurus, rhIDS was expressed at the highest levels observed among the other FGE sources. Co-expression with FGE derived from Hemicentrotus pulcherrimus and Mycobacterium tuberculosis demonstrated lower expression levels of IDS. Low IDS expression was noted in cultivations co-expressing Streptomyces coelicolor derived FGE.

### Example 5

### Analysis of the Activity of Y. lipolytica-Expressed Recombinant Human (rhIDS) Derived from a Strain Co-Expressing FGE from Different Origins

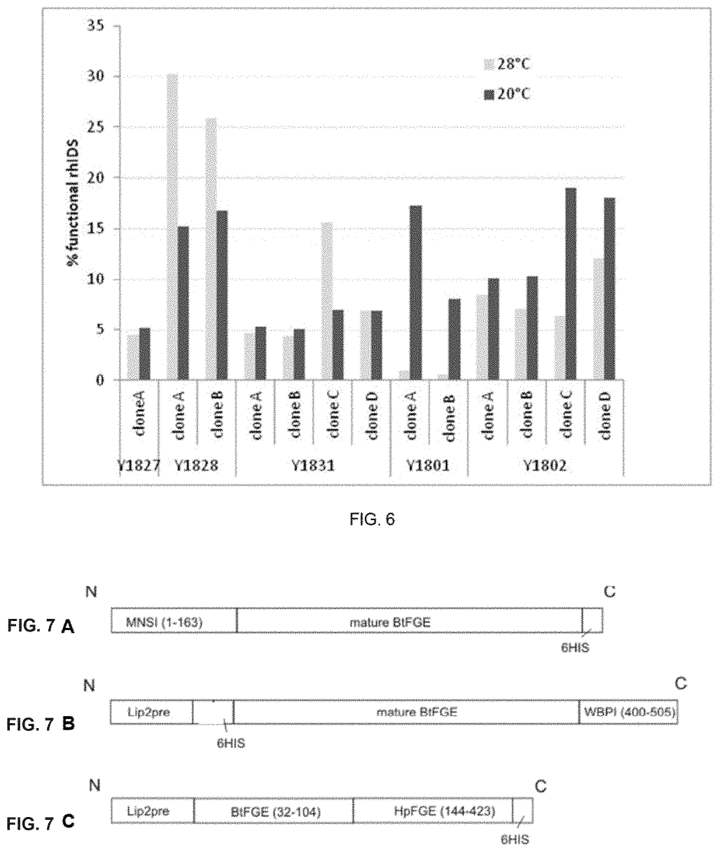

To compare and evaluate the level of production and secretion of human IDS among different recombinant Y. lipolytica strains, a fluorogenic activity assay using 4-methylumbelliferyl-alpha-L-iduronide-2-sulphate (4MU) and an ELISA quantification were employed. The activity of lysosomal iduronate 2-sulfatase was assayed using fluorogenic 4MU glycoside derivatives as a substrate, as described previously (Voznyi et al (2001), Journal of Inherited Metabolic Disease, 24, 675-680). The results are summarized in Table 4 and FIG. 6. Production of human IDS under oleic acid induction conditions at 28° C. and 20° C. was evaluated in 24 deep-well cultivation.

As shown in Table 4, several clones for each rFGE were tested. Percentage functional rhIDS was calculated as a ratio between the active rhIDS as determined in fluorogenic assay versus the total secreted human IDS as determined in sandwich ELISA. In both tests, the standard curves were generated using commercial elaprase. ELISA was performed on non-buffer exchanged samples, whereas activity was measured on buffer-exchanged samples.

TABLE 4
Expression of Active IDS in Y. lipolytica Strains Co-Expressing rhIDS and rFGE from
Different Origins

| | | 24 deep well plate | | 24 deep well plate | | | |
|---|---|---|---|---|---|---|---|
| | | cultivation at 28° C. | | cultivation at 20° C. | | % | % |
| | | μg/ml | μg/ml | μg/ml | μg/ml | functional | functional |
| | | active | rhIDS | active | rhIDS | rhIDS | rhIDS |
| | | rhIDS | (ELISA) | rhIDS | (ELISA) | (28° C.) | (20° C.) |
| OXYY1827 | clone A | 0.821 | 18.28031 | 0.633 | 12.40558 | 4.491172 | 5.102541 |
| OXYY1828 | clone A | 5.34 | 17.68521 | 1.972 | 12.98924 | 30.19473 | 15.18179 |
| | clone B | 5.168 | 19.99695 | 2.642 | 15.78164 | 25.84394 | 16.74097 |
| OXYY1831 | clone A | 1.255 | 26.92836 | 0.824 | 15.50698 | 4.660514 | 5.313736 |
| | clone B | 0.466 | 10.70039 | 0.365 | 7.221332 | 4.354982 | 5.054469 |
| | clone C | 0.362 | 2.317464 | 0.218 | 3.11284 | 15.62052 | 7.00325 |
| | clone D | 0.249 | 3.616388 | 0.288 | 4.200046 | 6.885323 | 6.857068 |
| OXYY1801 | clone A | 0.213 | 23.17082 | 0.723 | 4.200046 | 0.919259 | 17.2141 |
| | clone B | 0.123 | 24.29236 | 0.527 | 6.557565 | 0.506332 | 8.03652 |
| OXYY1802 | clone A | 0.814 | 9.605554 | 0.856 | 8.434424 | 8.474264 | 10.14888 |
| | clone B | 0.719 | 10.1091 | 0.649 | 6.305791 | 7.112402 | 10.29213 |
| | clone C | 0.842 | 13.09224 | 1.218 | 6.420233 | 6.43129 | 18.97127 |
| | clone D | 0.959 | 7.942321 | 1.262 | 6.992447 | 12.07456 | 18.04805 |

All Y. lipolytica strains co-expressing rFGE with rhIDS resulted in the expression of active rhIDS. Strains co-expressing Bos taurus (OXYY1828) demonstrated the strongest activity of rhIDS. This was particularly noted in stains cultivated at 28° C. In strains co-expressing Hemicentrotus pulcherrimus derived FGE (OXYY1801) a drastic increase in IDS-activity was seen when strains were grown at 20° C. instead of 28° C.

### Example 6

### Coexpression of hPDI in a Strain Expressing rhIDS and FGE

The present inventors considered that PDI co-expression in yeast could yield higher levels of active, secreted type I sulfatases in Y. lipolytica.

The LIP2 pre leader sequence was fused to the mature hPDI sequence (accession number NP_000909). A HDEL tetrapeptide was fused at the C-terminus to allow targeting to the ER. The complete protein sequence of the engineered protein is given below (SEQ ID NO 21; corresponding nucleic acid sequence set forth in SEQ ID NO: 22).

The PDI gene was synthesized and codon-optimized for Y. lipolytica expression and flanked by BamHI and AvrII for cloning into the expression vector under the control of the inducible POX2 promoter. The PDI-expressing plasmid was transformed into the rhIDS-FGE coexpressing strains using random integration and a dominant hygromycin marker. The strain construction overview of rhIDS expressing Y. lipolytica strains, co-expressing hPDI and FGE from different origin is shown in Table 5.

TABLE 5
Y. lipolytica Strains Co-Expressing Human
rIDS, rPDI and rFGE from Different Source

| rhIDS | rhFGE | |
|---|---|---|
| strains | coexpression | Strain genotype |
| OXYY1827 | humanFGE | MATA, leu2-958, ura3-302, xpe2-322, ade2- |
| (T126) | | 844, Sc suc2, Δoch1, URA3 ::POX2-MNN4, |
| | | OCH1 ::Hp4d-MNN4, POX2-Lip2pre- |
| | | hIDS:URA3Ex::zeta, Hp4d-Lip2pre- |
| | | hFGE:Leu2Ex::zeta, POX2-Lip2pre- |
| | | hFGE:Ade2Ex::zeta, POX2-Lip2pre- |
| | | hPDI:HygEx::zeta |
| OXYY1803 | BtFGE | MATA, leu2-958, ura3-302, xpe2-322, ade2- |
| (T156) | | 844, ΔSc suc2, Δoch1, URA3 ::POX2-MNN4, |
| | | OCH1 ::Hp4d-MNN4, POX2-Lip2pre- |
| | | hIDS:URA3Ex::zeta, Hp4d-Lip2pre- |
| | | BtFGE:Leu2Ex::zeta, POX2-Lip2pre- |
| | | BtFGE:Ade2Ex::zeta, POX2- Lip2pre- |
| | | hPDI:HygEx::zeta |
| OXYY1844 | ScFGE | MATA, leu2-958, ura3-302, xpe2-322, ade2-844, |
| (T157) | | ΔSc suc2, Δoch1, URA3 ::POX2-MNN4, OCH1 |
| | | ::Hp4d-MNN4, POX2-Lip2pre-hIDS:URA3Ex::zeta, |
| | | Hp4d -Lip2pre-ScFGE:Leu2Ex::zeta, POX2- |
| | | Lip2pre-ScFGE:Ade2Ex::zeta, POX2- Lip2pre- |
| | | hPDI:HygEx::zeta |
| OXYY1846 | HpFGE | MATA, leu2-958, ura3-302, xpe2-322, ade2- |
| (T158) | | 844, ΔSc suc2, Δoch1, URA3 ::POX2-MNN4, |
| | | OCH1 ::Hp4d-MNN4, POX2-Lip2pre- |
| | | hIDS:URA3Ex::zeta, Hp4d- Lip2pre- |
| | | HpFGE:Leu2Ex::zeta, POX2-Lip2pre- |
| | | HpFGE:Ade2Ex::zeta, POX2- Lip2pre- |
| | | hPDI:HygEx::zeta |
| OXYY1848 | MtFGE | MATA, leu2-958, ura3-302, xpe2-322, ade2- |
| (T159) | | 844, ΔSc suc2, Δoch1, URA3 ::POX2-MNN4, |
| | | OCH1 ::Hp4d-MNN4, POX2-Lip2pre- |
| | | hIDS:URA3Ex::zeta, Hp4d-Lip2pre- |
| | | MtFGE:Leu2Ex::zeta, POX2-Lip2pre- |
| | | MtFGE:Ade2Ex::zeta, POX2- Lip2pre- |
| | | hPDI:HygEx::zeta |

Each rhIDS strain had one rhIDS coding sequence copy co-expressed with 2 copies of either human, Bos taurus (Bt), Streptomyces coelicolor (Sc), Hemicentrotus pulcherrimus (Hp) or Mycobacterium tuberculosis (Mt) rFGE coding sequences. One rFGE was expressed under hp4d promoter while the other was expressed under POX2 promoter. Additionally, one POX2 driven hPDI coding sequence was expressed in each strain.

### Example 7

### Determination of FGly Conversion Using Nano-LC MS

Recombinant human IDS (rhIDS) produced in Y. lipolytica was treated with PNGaseF to remove N-glycans and separated on a SDS-PAGE gel. Proteins in excised gel slices were digested overnight with trypsin and followed by reduction with dithiothreitol and alkylation with iodoacetamide. The latter adds a carbamidomethyl group to the free cysteine residues and prevents the reformation of disulfide bridges. Trypsin cleaves the protein C-terminally of arginine and lysine residues. The resulting peptides were subsequently extracted from the gel and subject to nano-ultra-high pressure liquid chromatography (nano-UHPLC) connected to high-resolution tandem mass spectrometry (hybrid quadrupole time-of-flight—Q-TOF). A ThermoScientific/Dionex UHPLC system and an Agilent Technologies 6540 Q-TOF mass spectrometer were used. Separation was performed on a nano-column with an internal diameter of 75 μm and a length of 15 cm packed with sub 2 μm C18 particles. Injected peptides were eluted from the column at a flow rate of 300 nl/min using a 0.1% formic acid/acetonitrile gradient. Separated peptides were converted to gas-phase ions using a coated nanospray needle with an 8 μm tip maintained at 2000 V. Quadrupole time-of-flight measurement subsequently allowed the derivation of the m/z values of the intact peptides and the fragments thereof at high mass accuracy (<10 ppm). The formylglycine modified peptide derived from the peptide with the amino acid sequence SPNIDQLASHSLLFQNAFAQQAVCAPSR (cysteine residue that is subject to formylglycine conversion is underlined) could be quantified relative to the non-modified alkylated peptide by extracting, respectively, the triply charged ions at 999,1728 and 1024,1775 at an extraction window of 20 ppm and by determining the peak area following peak smoothing and integration. Identity was confirmed by obtaining the m/z values of the fragments generated by collision induced dissociation.

The results are shown in Table 6. Production of rhIDS under oleic acid inducting condition for 72 h was performed at 28° C. (except Hemicentrotus pulcherrimus-derived clone OXYY1801) in 24 deep-well cultivation unless stated differently in Table 6. Some strains were grown in duplicate (§). Strains co-expressing Hemicentrotus pulcherrimus-derived FGE (OXYY1801) demonstrated a drastic increase in IDS-activity when grown at 20° C. as compared to 28° C. Unless otherwise indicated, all strains were grown at 28° C.

TABLE 6
Conversion of the Formylglycine Residue in
IDS Expressed in Y. lipolytica Strains

| rhIDS production strain (all | % FGly |
|---|---|
| samples are fermentation samples) | conversion |
| T146 (OXYY1828; BtFGE) | 89.85 |
| T148 (OXYY1801; HpFGE) | 8.1 |
| T148 (OXYY1801; HpFGE) § | 3.72 |
| T148 (OXYY1801; HpFGE) (20° C.) | 68.14 |
| T146 (OXYY1828; BtFGE) § | 92.69 |

FGE derived from different organisms was concluded to be active when recombinantly expressed in Y. lipolytica strains. The derived FGEs analyzed were shown to convert the cysteine residues in the active site of the IDS protein to formylglycine. Recombinant FGE sourced from Bos taurus (T146; OXYY1828) was observed to be the most active of the FGEs from the other organisms that were analyzed. It was further concluded that the conversion rate to formylglycine was higher when the strains were cultivated in a fermenter. This is likely attributed to the higher partial oxygen pressure in a bioreactor as compared to alternative growth conditions which utilize a shake flask or a 24-well cultivation plate.

It was recently shown that formylglycine is easily hydrated with the formation of a geminal diol (Rabuka et al. (2012), Nat Protoc 7(6), 1052-1067) and that the aldehyde group in formylglycine can interact with the N-terminus of the peptide with the formation of a Schiff base resulting in a water loss (Grove et al. (2008) Biochemistry, 47(28), 7523-7538). Therefore, the the data shown in Table 6 were re-computed taking this geminal diol formation and water loss into account. The same bioreactor samples from OXYY1828 and OXYY1801 strains were re-analyzed.

TABLE 7
Re-analysis of samples shown in Table 6

| | | % FGly | % FGly |
|---|---|---|---|
| Bioreactor | rhIDS | conversion | conversion-re- |
| sample | production strain* | (from Table 6) | analyzed samples |
| DG29U5#7 | OXYY1828; BtFGE | 89.85 | 95.8 |
| DG29U7#7 | OXYY1801; HpFGE | 8.1 | 19.2 |
| DG33U1#6 | OXYY1801, HpFGE | 3.72 | 8.3 |
| DG33U3#6 | OXYY1801; HpFGE | 68.14 | 84.5 |
| | (20° C.) | | |
| DG33U8#6 | OXYY1828; BtFGE | 92.69 | 97.1 |
| DG33U6#6 | OXYY1803; BtFGE | 79.48 | 90.3 |
| | and hPDI | | |

Re-evaluation of the data confirmed that some of the formylglycine is indeed hydrated to the geminal diol. No Schiff base formation could be detected. The high rhIDS FGly conversion levels obtained by FGE that was sourced from Bos taurus (OXYY1828) was confirmed, as well as the low (<20%) FGly conversion levels obtained by FGE sourced from Hemicentrotus pulcherrimus (OXYY1801) when the Y. lipolytica strain was grown at 28° C. At 20° C. HpFGE enabled high FGly conversion.

The accuracy of the above-described nano-LC-MS method was further improved by incorporating a cation exchange chromatography purification step of the rhIDS. The results that were previously obtained (with rhIDS derived from gel slices) on Y. lipolytica coexpression of rhIDs and BtFGE were confirmed using this improved method. Such an experiment showed complete conversion (100%, with a detection limit of −0.5%) of Cys->FGly in rhIDS when BtFGE was coexpressed as a single POX2 driven copy. It was also confirmed that carboxymethylation of free cysteine residues occurred and that the 100% formylglycine incorporation detection was not sample preparation related.

### Example 8

### Determination of FGly Conversion in Sulfamidase Produced in Y. lipolytica Using Nano-LC MS

A Y. lipolytica strain was constructed that expressed recombinant human sulfamidase (rSGSH) (SEQ ID NO: 24; corresponding nucleic acid sequence set forth in SEQ ID NO: 25) and co-expressed BtFGE (1 copy, POX2 driven) and hPDI (1 copy, POX2 driven). A strain expressing rSGSH alone and a strain expressing rSGSH in combination with BtFGE without hPDI were also constructed.

These strains were grown in 24-well plates as described in Example 5. The supernatant was analyzed on SDS-PAGE and a gel slice containing SGSH was isolated for MS analysis. The results of the analysis are shown in Table 8.

TABLE 8
Conversion of the Formylglycine Residue in
rSGSH Expressed in Y. lipolytica strains

| Sample | SGSH production strain (all samples | % FGly |
|---|---|---|
| No. | are fermentation samples) | conversion |
| 1 | SGSH (POX) + BtFGE | 95.4 |
| | (POX) + hPDI (POX) | |
| 2 | SGSH (POX) + BtFGE | 80.4 |
| | (POX) + hPDI (POX) | |
| 3 | SGSH (POX) + BtFGE | 93.4 |
| | (POX) + hPDI (POX) § | |
| 4 | SGSH (POX) + BtFGE | 86.1 |
| | (POX) + hPDI (POX) § | |
| 5 | SGSH (POX) + BtFGE | 94.4 |
| | (POX & Hp4d) | |
| 6 | SGSH (POX) alone | 4.9 |

The first four samples shown in Table 8 are derived from the same strain run four times independantly in the bioreactor. The fifth sample was derived from a strain having two copies of BtFGE, one under the control of the POX2 inducible promoter and the other under the control of the Hp4d semi-constitutive promoter. Some strains were grown in duplicate (§). All strains were grown at 28° C. In the absence of an activating factor, 4.9% conversion to FGly was observed. This suggests the presence of a Y. lipolytica specific activation mechanism. It was concluded that FGEs from the different sources tested could convert cysteine to formylglycine in SGSH and thereby activated the enzyme. It was further concluded that the conversion rate to formylglycine was higher when the strains were cultivated in a fermentor.

### Example 9

### Use of Hemicentrotus pulcherrimus FGE (HpFGE) for rhIDS Activation in Y. lipolytica

The Cys->FGly conversion levels of a rhIDS expessing strain (OXYY1801) co-expressing HpFGE (Hemicentrotus pulcherrimus-derived FGE) at different growth temperatures was assessed. Strains co-expressing HpFGE (OXYY1801) demonstrated a drastic increase in IDS-activity when grown at 20° C. as compared to 28° C. Additionally, use of the Yarrowia MNS1 anchorage domain as a fusion with HpFGE in an attempt to improve ER retention of HpFGE was assessed. For the latter, fusion of the HpFGE to the transmembrane anchor of Yarrowia MNS1 (Accession: XP_502939.1) was performed to obtain correct localisation of HpFGE into the endoplasmic reticulum. Specifically, amino acids 1-163 of Y1MNS1 were fused N-terminally to the mature form of HpFGE. At the C-terminal end a 6HIS tag was added (SEQ ID NO: 35, corresponding coding sequence set out as SEQ ID NO: 36). The strain tested was designated FGE6.1.

The MNS1HpFGE coding sequence was synthesized and codon-optimized for expression within Y. lipolytica and was flanked by BamHI and AvrII restriction sites for cloning of the segment into an expression vector under the control of the inducible POX2 promoter or Hp4d promoter. The relevant constructs are designated OXYP3438 and OXYP3439, respectively. The plasmids were transformed into a Y. lipolytica strain expressing rhIDs (T116.22).

TABLE 9
Conversion of the Formylglycine Residue in strains of Y. lipolytica
co-expressing rIDS and rFGE cultivated in the bioreactor

| Strain ID | Strain description | *% FGly |
|---|---|---|
| OXYY1801 | 1c rhIDS, 2c HpFGE (POX/PHp4d) | 100 |
| (20° C.) | | |
| OXYY1801 | 1c rhIDS, 2c HpFGE (POX/PHp4d)−> | ND (FAILED) |
| (22° C.) | | |
| OXYY1801 | 1c rhIDS, 2c HpFGE (POX/PHp4d) | 100 |
| (24° C.) | | |
| OXYY1801 | 1c rhIDS, 2c HpFGE (POX/PHp4d) | 70.44 |
| (26° C.) | | |
| FGE6.1 | 1c rhIDS, 2c MNS1-HpFGE | 0 |

*FGly conversion of cation exchange chromatography purified samples (LC-MS)

The data obtained with these strains are shown in Table 9. As was previously observed, full conversion was detected when a Y. lipolytica strain co-expressing HpFGE was grown at 20° C. Also at 24° C., conversion was complete. When grown at higher temperature (26° C.) the conversion decreased to 70%. At 28° C. the conversion was less than 20%. It therefore seems likely the catalytic temperature optimum of HpFGE differs from that of the other tested FGEs.

For the MNS1-HpFGE strain (FGE6.1) conversion of Cys->FGly as determined by LC-MS was shown to be 0% at 28° C. This could be due to low expression or strongly reduced catalytic activity at 28° C. as was observed for the HDEL fusion protein.

### Example 10

### Localization of Mature rFGE to the Endoplasmic Reticulum (ER) by Fusion with the Anchorage Domain of Yarrowia lipolytica MNS1 Mannosidase

Fusions of rFGEs to the transmembrane anchorage domain of Yarrowia lipolytica MNS1 (Accession: XP_502939.1) were used to obtain localization of the rFGEs into the endoplasmic reticulum and reduce FGE secretion as was observed for HDEL tagged BtFGE. In order to do this, an expression vector containing a coding nucleotide sequence encoding a fusion polypeptide consisting of, N-terminus to C-terminus, amino acids 1-163 of MNS1 (SEQ ID NO: 26), a mature FGE (e.g., BtFGE), and a hexahistidine (6HIS) (FIG. 7A) was generated (SEQ ID NO: 37, corresponding coding sequence set out as SEQ ID NO: 38). It is expected that when this fusion polypeptide is expressed in Yarrowia lipolytica cells, it is localized to the ER of the cells.

The MNS1-BtFGE coding sequence, which was synthesized and codon-optimized for expression within Y. lipolytica, are flanked by BamHI and AvrII restriction sites for cloning of the segment into an expression vector under the control of the inducible POX2 promoter or Hp4d promoter. The relevant constructs were designated OXYP3418 and OXYP3424, respectively.

In addition, an expression vector containing a coding nucleotide sequence encoding a fusion polypeptide consisting of, N-terminus to C-terminus, amino acids 1-163 of MNS1 (SEQ ID NO: 26), a novel mature ClFGE from Columba livia (Rock dove), and a c-myc tag, was generated (SEQ ID NO: 67, corresponding coding sequence set out as SEQ ID NO: 68). It is expected that when this fusion polypeptide is expressed in Yarrowia lipolytica cells, it is localized to the ER of the cells.

The MNS1-ClFGE coding sequence, which was synthesized and codon-optimized for expression within Y. lipolytica, are flanked by BamHI and AvrII restriction sites for cloning of the segment into an expression vector under the control of the inducible POX2 promoter or Hp4d promoter.

### Example 11

### Localization of Mature rFGE to the Endoplasmic Reticulum (ER) by Fusion with the Anchorage Domain of Yarrowia lipolytica WBP1

Fusions of rFGEs to the transmembrane anchorage domain of Yarrowia lipolytica WBP1 (Accession: XP_502492.1) (Accession: XP_502939.1) to obtain localization of the rFGEs into the endoplasmic reticulum were generated. In order to do this, an expression vector containing a coding nucleotide sequence encoding a fusion polypeptide consisting of, N-terminus to C-terminus, the Lip2 signal sequence, a hexahistidine (6HIS) tag, a mature FGE (e.g., BtFGE), and the C-terminal 118 amino acids (amino acids 400-505 of XP_502492.1) of Yarrowia lipolytica WBP1 (SEQ ID NO: 28) (FIG. 7B) was generated. It is expected that when this fusion polypeptide is expressed in Yarrowia lipolytica cells, it is localized to the ER of the cells.

The WBP1-BtFGE coding sequence, which was synthesized and codon-optimized for expression within Y. lipolytica, are flanked by BamHI and AvrII restriction sites for cloning of the segment into an expression vector under the control of the inducible POX2 promoter or Hp4d promoter. Relevant constructs are designated OXYP3422 and OXYP3428, respectively.

### Example 12

### Production of a Construct Encoding Chimeric Protein Consisting of the N-Terminal End of BtFGE Fused to the C-Terminal End of HpFGE

A construct encoding a chimeric protein consisting of of the N-terminal end of BtFGE (amino acids 32-104 of NP_001069544, fused to the C-terminal end of HpFGE (amino acids 144-423 of BAJ83907) was generated. The Lip2 leader was fused to the N-terminal end of the chimeric coding sequence. At the C-terminus a 6HIS tag was added, followed by the HDEL tetrapeptide. A schematic representation of the protein is given in FIG. 7C.

The entire coding sequence, which was synthesized and codon-optimized for expression within Y. lipolytica, is flanked by BamHI and AvrII restriction sites for cloning of the segment into an expression vector under the control of the inducible POX2 promoter or Hp4d promoter. Relevant constructs are designated OXYP3420 and OXYP3426, respectively.

### Example 13

### Bioreactor Fermentation Expression Analysis of Fusion Proteins of Mature rFGE Designed to Localize to the Endoplasmic Reticulum (ER)

The strains of Y. lipolytica co-expressing rIDS and rFGE successfully cultivated in a bioreactor (Dasgip 37) are described in Table 10 below.

TABLE 10
Strains of Y. lipolytica co-expressing rIDS and
rFGE successfully cultivated in a bioreactor

| Unit | Strain ID | strain description |
|---|---|---|
| 1 | OXYY1818* | 1 copy rhIDS, 2 copies ChFGE |
| | | (POX/Hp4d) -20° C. |
| 2 | OXYY1818 | 1 copy rhIDS, 2 copiesChFGE |
| | | (POX/Hp4d) -28° C. |
| 3 | Y3035+* | 2 copy SGSH-5, 2 copies HpFGE |
| | | (POX/Hp4d) -20° C. |
| 4 | Y3035+ | 2 copy SGSH-5, 2 copies HpFGE |
| | | (POX/Hp4d) -28° C. |
| 5 | OXYY1822 | 1 copy rhIDS, 2 copies BtFGE- |
| | | WBPI (POX/Hp4d) |
| 6 | OXYY1826 | 1 copy rhIDS, 2 copies BtFGE- |
| | | MNS1 (POX/Hp4d) |
| 7 | OXYY1798 + | 1 copy rhIDS, 1 copy BtFGE |
| | hPDI | (POX), 1 copy hPDI (POX) |
| 8 | OXYY1798 | 1 copy rhIDS, 1 copy BtFGE (POX) |

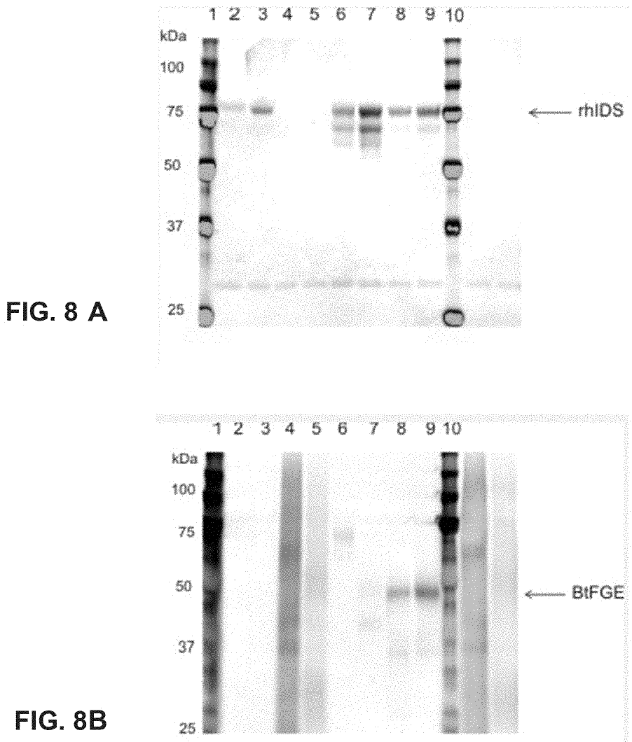

FIG. 8A shows the expression analysis (by Western blot with a rabbit anti-human IDS antiserum) of rhIDS from strains co-expressing rhIDS (1 copy, POX2 driven) and rFGE (1 copy POX2 driven and 1 copy Hp4d driven) grown under fed-batch fermentation. The Y. lipolytica-produced IDS is visible at an approximate MW of 76 kDa. The supernatant was analyzed for six rIDS expressing strains at the endpoint of the fermentation. Lane 1 is the MW Marker; lane 2 is ChFGE (the chimeric protein described in Example 12) co-expressed at 20° C.; lane 3 is ChFGE co-expressed at 28° C.; lane 6 is BtFGE-WBP1 co-expression; lane 7 is BtFGE-MNS1 co-expression; and lanes 8-9 are the control strains co-expressing BtFGE-HDEL (1 copy, POX2 driven). Varying levels of rhIDS were detected, with the highest levels obtained for the MNS1-BtFGE coexpression strain (lane 7). Degradation is present mostly in the WBPI-BtFGE and MNS1-BtFGE coexpression strains (lane 6 and lane 7 respectively).

FIG. 8B shows expression analysis of rFGE by Western blot using anti-his antibody (A00186-100, Genscript). The contents in each lane correspond to those in FIG. 8A. Small amounts of BtFGE were shown to leak into the media for Units 7 and 8 (1 copy PDX-driven expression of BtFGE) (lanes 8 and 9). However, in the case of the chimeric protein-expression constructs (lanes 2 and 3) no FGE leaked into the medium. For the WBP1 and MNS1-fusions only very low amounts of FGE leaked into the medium (lanes 6 and 7 respectively).

To compare and evaluate the level of production and secretion of human IDS among different recombinant Y. lipolytica strains, a fluorogenic activity assay using 4-methylumbelliferyl-alpha-L-iduronide-2-sulphate (4MU) and an ELISA quantification were employed. The activity of lysosomal iduronate 2-sulfatase was assayed using fluorogenic 4MU glycoside derivatives as a substrate, as described previously (Voznyi et al. (2001) J Inherit Metab Dis, 24(6), 675-680). Percentage functional rhIDS was calculated as a ratio between the active rhIDS as determined in fluorogenic assay versus the total secreted human IDS as determined in sandwich ELISA. In both tests, the standard curves were generated using commercial ELAPRASE®. Results are shown in Table 11.

TABLE 11
Conversion of the Formylglycine Residue in strains
of Y. lipolytica endoplasmic reticulum (ER)
fusion constructs cultivated in the bioreactor

| | rhIDS | | |
|---|---|---|---|
| | concentration | % | % FGly |
| Sample | (ng/ml) | active | (LC-MS) |
| 1 copy rhIDS, 2 copy ChFGE | 6065 | 0 | ND |
| (POX/Hp4d) -20° C.* | | | |
| 1 copy rhIDS, 2 copy ChFGE | 9268 | 0 | ND |
| (POX/Hp4d) -28° C. | | | |
| 1 copy rhIDS, 2 copy BtFGE- | 13078 | 124 | 89.15 |
| WBPI (POX/Hp4d) | | | |
| 1 copy rhIDS, 2 copy BtFGE- | 27542 | 98 | 99.5 |
| MNS1 (POX/Hp4d) | | | |
| 1 copy rhIDS, 1 copy BtFGE | 14620 | 121 | 100 |
| (POX), 1 copy hPDI (POX) | | | |
| 1 copy rhIDS, 1 copy BtFGE (POX) | 12534 | 129 | 100 |

In conclusion, a high level of activity and almost full Cys->FGly conversion was obtained when mature BtFGE protein was fused to MNS1 or WBP1 anchorage domains. Reduced leakage of the rFGE into the supernatant was observed when BtFGE was fused to MNS1 or WBP1 anchorage domains. Co-expression of BtFGE-MNS1 appeared to result in an increased rhIDS secetory level. Co-expression of WBP1- and MNS1-BtFGE resulted in increased proteolysis.

In a follow-up analysis carried out under the same conditions described above, two strains containing (i) two copies of rhIDS and one copy of BtFGE (POX2 driven) and (ii) one copy of rhIDS and 2 copies of BtFGE-MNS1 (one driven by POX2 and the other by Hp4d), gave 101% activity (with 100% FGly conversion at a detection limit of −0.5%) and 81.5% activity (with 100% FGly conversion), respectively.

### Example 14

### FGEs from Additional Species for Co-Expression in Y. lipolytica

A number of additional human FGE homologues were identified and and tested for their ability to activate rhIDs in Yarrowia lipolytica cells. A summary of the FGEs and their accession numbers is shown in Table 12.

TABLE 12
Overview of additional FGEs

| FGE origin | Accession No. |
|---|---|
| Gray short-tailed opossum | GI: 126336367 |
| (Monodelphis domestica) | |
| Rock Dove (Columba livia) | GI: 543740918 |
| Chinese tree shrew (Tupaia chinensis) | GI: 444707484 |
| Red junglefow (Gallus gallus) | GI: 363738801 |
| Mountain pine beetle | GI: 478257082 |
| (Dendroctonus ponderosa) | |

Mature sequences of the FGE's were fused at the N-terminus to the Lip2pre as a leader sequence (MKLSTILFTACATLAAA) (SEQ ID NO: 5). To the C-terminal end a 6His (HHHHHH) (SEQ ID NO: 7), followed by a HDEL tetrapeptide was fused (HDEL) (SEQ ID NO: 1). The amino acid sequences of the rFGE fusion proteins that were coexpressed in a Y. lipolytica strain expressing rhIDS are set out as SEQ ID NOs: 53, 55, 57, 59 and 61 (corresponding nucleic acid sequences SEQ ID NOs: 54, 56, 58, 60 and 62 respectively). The amino acid sequences of the corresponding mature FGEs are set out as SEQ ID NOs: 43, 45, 47, 49 and 51 (corresponding nucleic acid sequences SEQ ID NOs: 44, 46, 48, 50 and 52 respectively).

All FGE fusion coding sequences were synthesized and codon-optimized for expression within Y. lipolytica and were flanked by BamHI and AvrII restriction sites for cloning of the segment into an expression vector under the control of the inducible POX2 promoter or Hp4d promoter. A summary of these FGE co-expression strains is shown in Table 13. Each strain carries one copy of the rhIDS coding sequence co-expressed with two copies of either Tupaia chinensis (Tup), Monodelphis domestica (Md), Gallus gallus (Gg), Dendroctonus ponderosa (Dp) or Columba livia (Cl) rFGE coding sequence. In each strain, the two FGE copies are expressed under the POX2 promoter.

TABLE 13
Summary of the additional FGE co-expression strains

| | rFGE | |
|---|---|---|
| Strain ID | expressed | Strain genotype |
| OXYY3084 | TupFGE | MATA, leu2-958, ura3-302, xpe2-322, |
| | | ade2-844, ΔSc suc2, Δoch1, , URA3 :: |
| | | POX2-MNN4, OCH1 ::Hp4d-MNN4, |
| | | POX2-Lip2pre-hIDS:URA3Ex::zeta, |
| | | POX2-Lip2pre-TupFGE:Leu2Ex::zeta, |
| | | POX2-Lip2pre-TupFGE:Ade2Ex::zeta |
| OXYY3085 | MdFGE | MATA, leu2-958, ura3-302, xpe2-322, |
| | | ade2-844, ΔSc suc2, Δoch1, , URA3 :: |
| | | POX2-MNN4, OCH1 ::Hp4d-MNN4, |
| | | POX2-Lip2pre-hIDS:URA3Ex::zeta, |
| | | POX2-Lip2pre-MdFGE:Leu2Ex::zeta, |
| | | POX2-Lip2pre-MdFGE:Ade2Ex::zeta |
| OXYY3086 | GgFGE | MATA, leu2-958, ura3-302, xpe2-322, |
| | | ade2-844, ΔSc suc2, Δoch1, , URA3 :: |
| | | POX2-MNN4, OCH1 ::Hp4d-MNN4, |
| | | POX2-Lip2pre-hIDS:URA3Ex::zeta, |
| | | POX2-Lip2pre-GgFGE:Leu2Ex::zeta, |
| | | POX2-Lip2pre-GgFGE:Ade2Ex::zeta |
| OXYY3087 | DpFGE | MATA, leu2-958, ura3-302, xpe2-322, |
| | | ade2-844, ΔSc suc2, Δoch1, , URA3 :: |
| | | POX2-MNN4, OCH1 ::Hp4d-MNN4, |
| | | POX2-Lip2pre-hIDS:URA3Ex::zeta, |
| | | POX2-Lip2pre-DpFGE:Leu2Ex::zeta, |
| | | POX2-Lip2pre-DpFGE:Ade2Ex::zeta |
| OXYY3088 | ClFGE | MATA, leu2-958, ura3-302, xpe2-322, |
| | | ade2-844, ΔSc suc2, Δoch1, , URA3 :: |
| | | POX2-MNN4, OCH1 ::Hp4d-MNN4, |
| | | POX2-Lip2pre-Lip2pre-ClFGE: |
| | | Ade2Ex::zeta |

Clonal selection was based on 24-well cultivation. Strains of Y. lipolytica co-expressing rIDS and rFGE were successfully cultivated in a bioreactor (Dasgip 43) as set out in Table 14.

TABLE 14
Summary of the novel FGE co-expression
strains cultivated in the bioreactor

| Unit | Srain | Description |
|---|---|---|
| 1 | OXYY3086 | 1c rhIDS (POX), 2c GgFGE (POX/POX) |
| 2 | OXYY3087 | 1c rhIDS (POX), 2c DpFGE (POX/POX) |
| 3 | OXYY3088 | 1c rhIDS (POX), 2c ClFGE (POX/POX) |
| 4 | OXYY3085 | 1c rhIDS (POX), 2c MdFGE (POX/POX) |
| 5 | OXYY3084 | 1c rhIDS (POX), 2c TupFGE (POX/POX) |
| 6 | OXYY3089 | 1c rhIDS (POX), 2c MNS1-HpFGE (POX/POX) |

Fairly constant expression levels of rhIDs were observed with the different strain backgrounds. Unit 4 (MdFGE; Monodelphis domestica) showed incresed levels of rhIDS, however increased levels of rhIDs degradation were also visible. A variable degree of FGE can be observed in the supernatant with strong leakage of FGE to the medium in MdFGE strain. This can be explained by saturation of the HDEL receptor leading to significant leakage of the FGE into the supernatant.

As shown in Table 15, 100% FGly conversion for rhIDS was obtained for co-expression with MdFGE (Monodelphis domestica), ClFGE (Columba livia) and TupFGE (Tupaia chinensis). GgFGE (Gallus gallus) and DpFGE (Dendroctonus ponderosa) co-expression resulted in incomplete Cys to FGly conversion. The activity data show the same trend, with high specific activity for TupFGE, ClFGE and MdFGE, intermediate activity for GgFGE and low activity for DpFGE.

TABLE 15
Overview of % activity and FGly conversion as
determined by LC-MS for the additional strains.

| | | % FGly |
|---|---|---|
| rFGE | % activity | (LC-MS) |
| GgFGE | 33 | 78 |
| DpFGE | 5 | 25 |
| ClGFE | 61 | 100 |
| MdFGE | 58 | 100 |
| TupFGE | 51 | 99 |

In summary, co-expression of three rFGE's, MdFGE, ClFGE and TupFGE resulted in complete or essentially complete conversion of FGly in rhIDS.

### Example 15

### Analysis of the Activity of rhIDS Obtained from a Recombinant Strain of Yarrowa lipolytica not Co-Expressing an rFGE

A recombinant Yarrowia lipolytica strain (T135) was constructed containing two PDX driven copies of a rhIDS coding sequence with the following genotype: Δoch1, URA3::POX2-MNN4, OCH1::Hp4d-MNN4, POX2-Lip2pre-hIDS::zeta, POX2-Lip2pre-hIDS::zeta. This strain contained no rFGE expressing nucleotide sequence. Production of rhIDS under oleic acid inducting condition was performed in a fermentor using standard protocol.

To compare and evaluate the level of production and secretion of rhIDS, a fluorogenic activity assay using 4-methylumbelliferyl-alpha-L-iduronide-2-sulphate (4MU) was employed. The activity of rhIDS in supernatant recovered from the culture was assayed as previously described Voznyi et al (2001), Journal of Inherited Metabolic Disease, 24, 675-680). The assay does not detect sulfamidase activity. Absorbances are summarized in Table 16. A control Yarrowia lipolytica strain was constructed that did not express rhIDS but expressed human sulfamidase (hSGSH) and co-expressed BtfGE (1 copy, POX2 driven) and hPDI (1 copy, POX2 driven). Clearly, elevated sulfatase activity could be observed in the supernatant of the rhIDS expressing strain, corresponding to 30 ng/ml of active rhIDS. Results therefore show from the low IDS activity in the control strain that expression of FGE is required for IDS activity.

TABLE 16
IDS activity (in absorbance units) secreted by a recombinant
strain of Yarrowia lipolytica producing rhIDS
versus a control strain expressing hSGSH.

| Supernatant | Strain T135 | Control Strain |
|---|---|---|
| dilution factor | (expressing rhIDS) | (not expressing rhIDS) |
| 10 | 2717 | 44 |
| 50 | 606 | 21 |
| 100 | 353 | 37 |

### Example 16

### Construction of Yarrowia lipolytica Strains Co-Expressing Human Endoplasmic Reticulum Resident Protein 44 (hERP44) and rFGE

Yarrowia lipolytica strains are constructed in which rFGEs (e.g., BtFGE) without a C-terminal HDEL signal sequence are co-expressed with hERp44. In order to do this, two expression vectors are made. The first contains a coding nucleotide sequence encoding a fusion polypeptide consisting of, N-terminus to C-terminus, the Lip2 signal sequence (SEQ ID NO: 6), and the mature form of hERp44 (SEQ ID NO: 30; Accession: CAC87611.1) with the C-terminal RDEL sequence replaced by a HDEL tetrapeptide (SEQ ID NO:1). The second vector contains a coding nucleotide sequence encoding a fusion polypeptide consisting of, N-terminus to C-terminus, the Lip2 signal sequence (SEQ ID NO: 6) and the mature form of an rFGE (e.g., BtFGE). It is expected that co-expression of the two expression vectors in Yarrowia lipolytica cells results in the localization of rFGE fusion polypeptide to the ER of the cells.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

SEQUENCES REFERRED TO IN THE APPLICATION

SEQ ID 1: HDEL tag
HDEL
SEQ ID 2: HDEL tag coding sequence
CACGACGAGCTG
SEQ ID 3: KDEL tag
KDEL
SEQ ID 4: DDEL tag
DDEL
SEQ ID NO 5: LIP2 leader sequence
MKLSTILFTACATLAAA
SEQ ID NO 6: LIP2 leader sequence; coding sequence
ATGAAGCTGTCTACTATTCTCTTTACTGCCTGCGCTACTCTCGCCGCTGCT
SEQ ID NO 7: Six Histidine (HIS) tag
HHHHHH
SEQ ID NO 8: Six Histidine (HIS) tag
CACCACCACCACCACCAC
SEQ ID NO 9; Human FGE mature protein
SQEAGTGAGAGSLAGSCGCGTPQRPGAHGSSAAAHRYSREANAPGPVPGERQLAHSKM
VPIPAGVFTMGTDDPQIKQDGEAPARRVTIDAFYMDAYEVSNTEFEKFVNSTGYLTEAE
KFGDSFVFEGMLSEQVKTNIQQAVAAAPWWLPVKGANWRHPEGPDSTILHRPDHPVLH
VSWNDAVAYCTWAGKRLPTEAEWEYSCRGGLHNRLFPWGNKLQPKGQHYANIWQGE
FPVTNTGEDGFQGTAPVDAFPPNGYGLYNIVGNAWEWTSDWWTVHHSVEETLNPKGP
PSGKDRVKKGGSYMCHRSYCYRYRCAARSQNTPDSSASNLGFRCAADRLPTMD
SEQ ID NO 10; Human FGE coding sequence of the mature protein
TCCCAGGAAGCCGGCACCGGAGCTGGTGCTGGTTCTCTGGCTGGATCGTGCGGATGT
GGCACTCCTCAGCGACCTGGAGCTCATGGCTCCTCTGCCGCTGCCCACCGATACTCT
CGAGAGGCTAACGCTCCTGGTCCTGTCCCCGGAGAGCGACAGCTCGCCCATTCTAAG
ATGGTGCCTATCCCCGCTGGAGTTTTCACCATGGGCACTGACGATCCTCAGATCAAG
CAGGACGGAGAGGCTCCTGCTCGACGAGTGACCATTGACGCCTTTTACATGGATGCT
TACGAGGTTTCGAACACTGAGTTCGAGAAGTTTGTCAACTCTACCGGATACCTGACT
GAGGCCGAGAAGTTCGGTGACTCGTTCGTGTTTGAGGGAATGCTCTCCGAGCAGGTC
AAGACCAACATCCAGCAGGCTGTGGCTGCCGCTCCTTGGTGGCTGCCCGTTAAGGG
AGCTAACTGGCGACACCCTGAGGGACCTGACTCCACCATTCTGCACCGACCTGATCA
TCCCGTCCTCCACGTGTCTTGGAACGACGCCGTTGCTTACTGTACCTGGGCTGGCAA
GCGACTGCCTACTGAGGCTGAGTGGGAGTACTCCTGCCGAGGCGGTCTGCATAACC
GACTCTTCCCTTGGGGCAACAAGCTCCAGCCCAAGGGTCAGCACTACGCCAACATCT
GGCAGGGCGAGTTTCCTGTGACCAACACTGGAGAGGACGGATTCCAGGGCACCGCT
CCTGTTGATGCTTTTCCCCCTAACGGTTACGGACTGTACAACATTGTCGGTAACGCTT
GGGAGTGGACCTCTGACTGGTGGACTGTTCACCATTCGGTCGAGGAGACCCTCAACC
CCAAGGGCCCTCCCTCTGGCAAGGATCGAGTCAAGAAGGGAGGCTCCTACATGTGC
CACCGATCTTACTGTTACCGATACCGATGCGCCGCTCGATCCCAGAACACCCCCGAC
TCGTCCGCCTCTAACCTGGGCTTCCGATGTGCCGCTGACCGACTGCCTACTATGGAC
SEQ ID NO 11; Streptomyces coelicolor FGE mature protein
MAVAAPSPAAAAEPGPAARPRSTRGQVRLPGGEFAMGDAFGEGYPADGETPVHTVRLR
PFHIDETAVTNARFAAFVKATGHVTDAERFGSSAVFHLVVAAPDADVLGSAAGAPWWI
NVRGAHWRRPEGARSDITGRPNHPVVHVSWNDATAYARWAGKRLPTEAEWEYAARG
GLAGRRYAWGDELTPGGRWRCNIWQGRFPHVNTAEDGHLSTAPVKSYRPNGHGLWNT
AGNVWEWCSDWFSPTYYAESPTVDPHGPGTGAARVLRGGSYLCHDSYCNRYRVAARS
SNTPDSSSGNLGFRCANDADLTSGSAAE
SEQ ID NO 12; Streptomyces coelicolor FGE coding sequence of the mature protein
ATGGCTGTTGCTGCTCCCTCGCCTGCTGCTGCTGCCGAGCCCGGTCCTGCTGCTCGAC
CCCGATCTACCCGAGGACAGGTGCGACTGCCTGGCGGTGAGTTCGCTATGGGCGAC
GCTTTTGGAGAGGGATACCCTGCCGATGGAGAGACCCCTGTGCACACTGTTCGACTC
CGACCCTTCCATATCGACGAGACCGCTGTTACTAACGCCCGATTCGCCGCTTTTGTC
AAGGCTACCGGACACGTGACTGATGCCGAGCGATTCGGCTCCTCTGCTGTTT
TTCATCTGGTCGTGGCCGCTCCCGACGCTGATGTCCTGGGCTCCGCTGCTGGAGCTC
CTTGGTGGATCAACGTTCGAGGTGCCCACTGGCGACGACCTGAGGGAGCTCGATCTG
ACATTACCGGTCGACCCAACCACCCTGTTGTCCATGTCTCCTGGAACGATGCTACCG
CTTACGCTCGATGGGCTGGAAAGCGACTGCCTACTGAGGCTGAGTGGGAGTACGCT
GCTCGAGGCGGCCTGGCTGGTCGACGATACGCTTGGGGAGACGAGCTCACCCCCGG
TGGACGATGGCGATGCAACATTTGGCAGGGACGATTCCCTCACGTCAACACCGCCG
AGGACGGCCATCTGTCCACTGCTCCCGTGAAGTCTTACCGACCTAACGGTCACGGAC
TCTGGAACACCGCCGGTAACGTCTGGGAGTGGTGTTCTGACTGGTTTTCGCCCACCT
ACTACGCCGAGTCTCCTACTGTCGACCCCCACGGACCTGGTACTGGAGCTGCTCGAG
TTCTGCGAGGCGGTTCGTACCTCTGCCATGACTCCTACTGTAACCGATACCGAGTGG
CCGCTCGATCGTCCAACACCCCCGACTCTTCGTCCGGCAACCTCGGTTTCCGATGCG
CCAACGATGCTGACCTGACTTCTGGATCTGCCGCTGAG
SEQ ID NO 13; Hemicentrotus pulcherrimus FGE mature protein
ENEDINQNISPTQSHTTATTEEELAEARGEEIDSDPTSEGSGAGEGCGCGSSALNRNHDE
DALGLALEENLHDHVQEGAALKYSREANDPISMDHPEANVGAFPRTNQMNFIEGGTFR
MGTDKAKIYLDGESPSRLVTLDPYYFDVYEVSNSEFELFVNTTSYITEAEKFGDSFVLEA
RISEEVKKDISQVVAAAPWWLPVKGAEWRHPEGPDSSISSRMDHPVTHISWNDATAYC
QWAGKRLPTEAEWENAARGGLNNRLFPWGNKLMPKDHHRVNIWQGEFPKVNTAEDG
YEGTCPVTAFEPNGYGLYNTVGNAWEWVADWWTTVHSPESQNNPVGPDEGTDKVKK
GGSYMCHISYCYRYRCEARSQNSPDSSACNLGFRCAATNLPEDIPCSNCNDSTP
SEQ ID NO 14; Hemicentrotus pulcherrimus FGE coding sequence of the mature protein
GAGAACGAGGACATCAACCAGAACATTTCGCCTACCCAGTCTCACACCACTGCCAC
CACTGAGGAAGAGCTCGCTGAGGCCCGAGGCGAGGAGATCGACTCCGATCCCACCT
CTGAGGGCTCTGGTGCTGGAGAGGGATGCGGTTGTGGCTCCTCTGCCCTGAACCGAA
ACCACGACGAGGATGCTCTGGGTCTCGCCCTGGAGGAGAACCTCCACGACCATGTT
CAGGAAGGCGCCGCTCTGAAGTACTCGCGAGAGGCTAACGACCCCATTTCTATGGA
TCATCCTGAGGCTAACGTCGGTGCCTTCCCCCGAACCAACCAGATGAACTTCATCGA
GGGCGGTACCTTTCGAATGGGAACTGACAAGGCCAAGATCTACCTGGATGGTGAAT
CTCCTTCCCGACTGGTGACCCTGGACCCTTACTACTTTGATGTTTACGAGGTCTCTAA
CTCGGAGTTCGAGCTCTTTGTTAACACCACTTCTTACATCACCGAGGCTGAGAAGTT
CGGTGACTCCTTTGTGCTGGAGGCCCGAATCTCTGAGGAAGTCAAGAAGGATATTTC
TCAGGTGGTGGCTGCTGCTCCTTGGTGGCTCCCCGTCAAGGGTGCTGAGTGGCGACA
CCCTGAGGGTCCTGACTCGTCCATCTCTTCGCGAATGGATCACCCCGTGACCCATAT
TTCCTGGAACGACGCTACTGCCTACTGTCAGTGGGCTGGAAAGCGACTCCCTACCGA
GGCTGAGTGGGAGAACGCTGCTCGAGGCGGCCTCAACAACCGACTGTTCCCCTGGG
GCAACAAGCTGATGCCTAAGGACCACCATCGAGTTAACATTTGGCAGGGAGAGTTC
CCCAAGGTCAACACCGCTGAGGACGGATACGAGGGCACCTGCCCCGTGACTGCCTT
TGAGCCTAACGGCTACGGTCTGTACAACACTGTGGGAAACGCTTGGGAGTGGGTTG
CCGACTGGTGGACCACTGTCCACTCGCCCGAGTCCCAGAACAACCCCGTCGGTCCTG
ACGAGGGAACCGATAAGGTCAAGAAGGGCGGCTCCTACATGTGCCATATCTCTTAC
TGTTACCGATACCGATGCGAGGCTCGATCTCAGAACTCGCCCGACTCCTCTGCCTGT
AACCTCGGCTTCCGATGCGCTGCCACCAACCTGCCTGAGGACATTCCTTGTTCTAAC
TGTAACGATTCCACTCCC
SEQ ID NO 15; Bos taurus FGE coding sequence mature sequence
AGGEEAGPEAGAPSLVGSCGCGNPQRPGAQGSSAAAHRYSREANAPGSVPGGRPSPPTK
MVPIPAGVFTMGTDDPQIKQDGEAPARRVAIDAFYMDAYEVSNAEFEKFVNSTGYLTE
AEKFGDSFVFEGMLSEQVKSDIQQAVAAAPWWLPVKGANWRHPEGPDSTVLHRPDHP
VLHVSWNDAVAYCTWAGKRLPTEAEWEYSCRGGLQNRLFPWGNKLQPKGQHYANIW
QGEFPVTNTGEDGFRGTAPVDAFPPNGYGLYNIVGNAWEWTSDWWTVHHSAEETINPK
GPPSGKDRVKKGGSYMCHKSYCYRYRCAARSQNTPDSSASNLGFRCAADHLPTTGAD
HLPTTG
SEQ ID NO 16; Bos taurus FGE coding sequence of the mature protein
GCCGGCGGCGAGGAAGCCGGACCTGAGGCCGGCGCTCCCTCTCTGGTTGGATCGTG
TGGATGTGGAAACCCCCAGCGACCTGGCGCTCAGGGTTCCTCTGCCGCTGCCCACCG
ATACTCTCGAGAGGCTAACGCTCCTGGCTCTGTCCCTGGAGGCCGACCCTCGCCCCC
TACCAAGATGGTTCCCATCCCTGCCGGCGTCTTCACCATGGGTACTGACGATCCTCA
GATCAAGCAGGACGGAGAGGCTCCTGCTCGACGAGTGGCTATTGACGCTTTTTACAT
GGATGCCTACGAGGTCTCTAACGCTGAGTTCGAGAAGTTTGTGAACTCGACCGGATA
CCTGACTGAGGCCGAGAAGTTCGGAGACTCCTTCGTTTTTGAGGGCATGCTCTCCGA
GCAGGTGAAGTCTGATATTCAGCAGGCTGTTGCTGCCGCTCCTTGGTGGCTGCCTGT
CAAGGGAGCTAACTGGCGACATCCCGAGGGTCCTGACTCCACCGTGCTGCACCGAC
CCGATCATCCTGTCCTCCACGTGTCTTGGAACGACGCCGTCGCTTACTGTACCTGGG
CTGGCAAGCGACTGCCTACTGAGGCTGAGTGGGAGTACTCTTGCCGAGGTGGACTG
CAGAACCGACTCTTCCCTTGGGGTAACAAGCTCCAGCCCAAGGGACAGCACTACGC
CAACATCTGGCAGGGAGAGTTTCCTGTGACCAACACTGGTGAAGACGGCTTCCGAG
GCACCGCTCCTGTTGATGCTTTTCCCCCTAACGGTTACGGACTCTACAACATCGTTGG
CAACGCCTGGGAGTGGACCTCCGACTGGTGGACTGTCCACCATTCTGCTGAGGAGA
CTATTAACCCCAAGGGTCCCCCTTCTGGAAAGGATCGAGTGAAGAAGGGCGGTTCG
TACATGTGCCACAAGTCCTACTGTTACCGATACCGATGCGCCGCTCGATCGCAGAAC
ACCCCCGACTCGTCCGCCTCCAACCTGGGATTCCGATGTGCCGCTGACCACCTGCCT
ACTACTGGA
SEQ ID NO 17; Mycobacterium tuberculosis FGE mature sequence
MLTELVDLPGGSFRMGSTRFYPEEAPIHTVTVRAFAVERHPVTNAQFAEFVSATGYVTV
AEQPLDPGLYPGVDAADLCPGAMVFCPTAGPVDLRDWRQWWDWVPGACWRHPFGR
DSDIADRAGHPVVQVAYPDAVAYARWAGRRLPTEAEWEYAARGGTTATYAWGDQEK
PGGMLMANTWQGRFPYRNDGALGWVGTSPVGRFPANGFGLLDMIGNVWEWTTTEFY
PHHRIDPPSTACCAPVKLATAADPTISQTLKGGSHLCAPEYCHRYRPAARSPQSQDTATT
HIGFRCVADPVSG
SEQ ID NO 18; Mycobacterium tuberculosis FGE coding sequence of the mature protein
ATGCTGACTGAGCTGGTTGACCTCCCTGGTGGTTCCTTCCGAATGGGATCTACCCGA
TTTTACCCCGAGGAGGCCCCTATCCACACTGTTACCGTCCGAGCCTTCGCTGTCGAG
CGACATCCCGTGACCAACGCTCAGTTCGCCGAGTTTGTTTCGGCTACTGGCTACGTG
ACCGTTGCTGAGCAGCCTCTGGACCCTGGACTCTACCCTGGAGTCGACGCTGCTGAT
CTGTGCCCTGGCGCTATGGTCTTCTGTCCTACCGCTGGTCCTGTGGACCTCCGAGATT
GGCGACAGTGGTGGGACTGGGTCCCTGGTGCTTGCTGGCGACACCCTTTTGGACGAG
ACTCCGATATTGCTGACCGAGCTGGACATCCTGTCGTGCAGGTGGCTTACCCTGATG
CCGTTGCTTACGCTCGATGGGCTGGTCGACGACTGCCTACTGAGGCTGAGTGGGAGT
ACGCTGCTCGAGGAGGTACCACTGCTACCTACGCTTGGGGTGACCAGGAGAAGCCT
GGAGGCATGCTGATGGCTAACACCTGGCAGGGACGATTCCCTTACCGAAACGATGG
AGCCCTCGGCTGGGTTGGTACCTCCCCTGTCGGACGATTCCCTGCTAACGGCTTTGG
TCTGCTCGACATGATCGGCAACGTGTGGGAGTGGACCACTACCGAGTTTTACCCCCA
CCATCGAATTGACCCCCCTTCTACTGCTTGCTGTGCTCCTGTTAAGCTCGCTACCGCT
GCTGATCCTACTATCTCGCAGACCCTGAAGGGTGGCTCCCACCTCTGCGCTCCCGAG
TACTGTCATCGATACCGACCCGCCGCTCGATCCCCTCAGTCTCAGGACACCGCCACT
ACCCACATTGGTTTTCGATGTGTTGCTGACCCTGTTTCGGGC
SEQ ID NO 19; human iduronate sulfatase mature sequence
SETQANSTTDALNVLLIIVDDLRPSLGCYGDKLVRSPNIDQLASHSLLFQNAFAQQAVCA
PSRVSFLTGRRPDTTRLYDFNSYWRVHAGNFSTIPQYFKENGYVTMSVGKVFHPGISSN
HTDDSPYSWSFPPYHPSSEKYENTKTCRGPDGELHANLLCPVDVLDVPEGTLPDKQSTE
QAIQLLEKMKTSASPFFLAVGYHKPHIPFRYPKEFQKLYPLENITLAPDPEVPDGLPPVAY
NPWMDIRQREDVQALNISVPYGPIPVDFQRKIRQSYFASVSYLDTQVGRLLSALDDLQL
ANSTIIAFTSDHGWALGEHGEWAKYSNFDVATHVPLIFYVPGRTASLPEAGEKLFPYLDP
FDSASQLMEPGRQSMDLVELVSLFPTLAGLAGLQVPPRCPVPSFHVELCREGKNLLKHF
RFRDLEEDPYLPGNPRELIAYSQYPRPSDIPQWNSDKPSLKDIKIMGYSIRTIDYRYTVWV
GFNPDEFLANFSDIHAGELYFVDSDPLQDHNMYNDSQGGDLFQLLMP
SEQ ID NO 20; human iduronate sulfatase coding sequence of the mature protein
TCTGAGACCCAGGCTAACTCGACTACTGACGCTCTGAACGTGCTCCTGATTATTGTT
GACGACCTGCGACCCTCCCTCGGTTGCTACGGTGACAAGCTGGTGCGATCTCCCAAC
ATCGACCAGCTCGCTTCTCACTCGCTGCTCTTCCAGAACGCCTTTGCTCAGCAGGCC
GTCTGCGCTCCTTCGCGAGTGTCCTTCCTGACCGGACGACGACCCGACACCACTCGA
CTCTACGATTTTAACTCCTACTGGCGAGTCCACGCCGGTAACTTCTCTACCATCCCTC
AGTACTTTAAGGAGAACGGATACGTGACTATGTCCGTGGGCAAGGTTTTCCACCCCG
GTATTTCCTCTAACCATACCGACGATTCTCCTTACTCCTGGTCTTTTCCCCCTTACCA
CCCCTCGTCCGAGAAGTACGAGAACACCAAGACTTGCCGAGGCCCTGACGGAGAGC
TGCATGCTAACCTGCTCTGTCCCGTCGACGTGCTGGATGTTCCTGAGGGAACCCTCC
CCGATAAGCAGTCCACTGAGCAGGCCATTCAGCTGCTCGAGAAGATGAAGACCTCG
GCCTCCCCCTTCTTTCTGGCTGTCGGCTACCACAAGCCCCATATCCCTTTCCGATACC
CTAAGGAGTTTCAGAAGCTGTACCCCCTCGAGAACATTACCCTGGCTCCCGACCCTG
AGGTTCCTGATGGTCTGCCTCCCGTGGCTTACAACCCTTGGATGGACATCCGACAGC
GAGAGGATGTGCAGGCCCTGAACATCTCCGTTCCCTACGGTCCCATTCCTGTCGACT
TCCAGCGAAAGATTCGACAGTCTTACTTTGCTTCTGTGTCGTACCTGGACACCCAGG
TTGGTCGACTGCTCTCCGCCCTCGACGATCTGCAGCTCGCCAACTCGACCATCATTG
CTTTCACTTCCGACCACGGATGGGCCCTGGGAGAGCATGGCGAGTGGGCTAAGTACT
CTAACTTCGACGTTGCCACCCACGTCCCTCTGATCTTTTACGTTCCTGGACGAACTGC
CTCCCTCCCTGAGGCTGGTGAAAAGCTGTTCCCTTACCTCGACCCCTTTGATTCCGCT
TCTCAGCTGATGGAGCCTGGCCGACAGTCTATGGACCTGGTCGAGCTCGTGTCGCTG
TTCCCCACCCTGGCTGGTCTGGCTGGCCTGCAGGTCCCTCCCCGATGCCCCGTGCCTT
CTTTCCACGTTGAGCTCTGTCGAGAGGGAAAGAACCTGCTCAAGCATTTCCGATTTC
GAGACCTGGAGGAAGACCCCTACCTCCCTGGCAACCCCCGAGAGCTGATCGCCTAC
TCCCAGTACCCCCGACCTTCTGACATTCCTCAGTGGAACTCTGACAAGCCCTCGCTC
AAGGATATCAAGATTATGGGCTACTCCATCCGAACCATTGACTACCGATACACTGTT
TGGGTCGGTTTCAACCCCGACGAGTTCCTGGCCAACTTTTCGGATATTCACGCTGGA
GAGCTGTACTTCGTCGACTCTGATCCCCTCCAGGACCATAACATGTACAACGACTCG
CAGGGCGGTGACCTCTTCCAGCTCCTGATGCCT
SEQ ID NO 21; human PDI mature sequence
DAPEEEDHVLVLRKSNFAEALAAHKYLLVEFYAPWCGHCKALAPEYAKAAGKLKAEG
SEIRLAKVDATEESDLAQQYGVRGYPTIKFFRNGDTASPKEYTAGREADDIVNWLKKRT
GPAATTLPDGAAAESLVESSEVAVIGFFKDVESDSAKQFLQAAEAIDDIPFGITSNSDVFS
KYQLDKDGVVLFKKFDEGRNNFEGEVTKENLLDFIKHNQLPLVIEFTEQTAPKIFGGEIK
THILLFLPKSVSDYDGKLSNFKTAAESFKGKILFIFIDSDHTDNQRILEFFGLKKEECPAVR
LITLEEEMTKYKPESEELTAERITEFCHRFLEGKIKPHLMSQELPEDWDKQPVKVLVGKN
FEDVAFDEKKNVFVEFYAPWCGHCKQLAPIWDKLGETYKDHENIVIAKMDSTANEVEA
VKVHSFPTLKFFPASADRTVIDYNGERTLDGFKKFLESGGQDGAGDDDDLEDLEEAEEP
DMEEDDDQKAV
SEQ ID NO 22; human PDI coding sequence of the mature protein
GACGCCCCCGAGGAAGAGGACCACGTCCTGGTCCTGCGAAAGTCTAACTTCGCCGA
GGCCCTGGCCGCCCACAAGTACCTGCTGGTCGAATTCTACGCCCCCTGGTGCGGCCA
CTGCAAGGCCCTCGCTCCCGAGTACGCCAAGGCCGCTGGCAAGCTGAAGGCCGAGG
GCTCTGAGATCCGACTGGCCAAGGTGGACGCCACCGAGGAATCTGACCTGGCCCAG
CAGTACGGCGTGCGAGGCTACCCCACCATCAAGTTCTTCCGAAACGGCGACACCG
CCTCTCCCAAGGAGTACACCGCCGGACGAGAGGCCGACGACATCGTGAACTGGCTG
AAGAAGCGAACCGGACCCGCCGCTACTACTCTGCCCGACGGCGCTGCCGCCGAGTC
TCTGGTCGAGTCCTCTGAGGTGGCCGTGATCGGCTTCTTCAAGGACGTCGAGTCTGA
CTCTGCCAAGCAGTTCCTGCAGGCCGCCGAGGCCATCGACGACATTCCCTTCGGCAT
CACCTCTAACTCTGACGTGTTCTCTAAGTACCAGCTGGACAAGGACGGCGTGGT
GCTGTTCAAGAAGTTCGACGAGGGCCGAAACAACTTCGAGGGCGAGGTGACCAAGG
AAAACCTGCTGGACTTCATCAAGCACAACCAGCTGCCCCTGGTGATCGAGTTCACCG
AGCAGACCGCCCCCAAGATTTTCGGCGGCGAGATCAAGACCCACATCCTGCTGTTTC
TGCCCAAGTCTGTGTCTGACTACGACGGCAAGCTGTCTAACTTCAAGACCGCCGCTG
AGTCTTTCAAGGGCAAGATCCTGTTCATCTTCATCGACTCTGACCACACCGACAACC
AGCGAATCCTCGAGTTCTTCGGCCTGAAGAAAGAAGAATGTCCCGCCGTCCGACTG
ATCACCCTCGAGGAAGAGATGACCAAGTACAAGCCCGAGTCTGAGGAACTGACCGC
CGAGCGAATCACCGAGTTCTGCCACCGATTCCTCGAGGGCAAGATCAAGCCCCACC
TGATGTCTCAGGAACTGCCCGAGGACTGGGATAAGCAGCCCGTGAAGGTGCTGGTG
GGCAAGAACTTCGAGGACGTGGCCTTCGACGAGAAGAAGAACGTTTTCGTCGAGTT
TTACGCTCCTTGGTGTGGACACTGTAAGCAGCTGGCCCCCATCTGGGACAAGCTGGG
CGAGACTTACAAGGACCACGAGAACATCGTGATCGCCAAGATGGACTCTACCGCCA
ACGAGGTCGAGGCCGTGAAGGTCCACTCGTTCCCCACCCTGAAGTTCTTTCCCGCCT
CTGCCGACCGAACCGTGATCGACTACAACGGCGAGCGAACCCTGGACGGCTTCAAG
AAGTTTCTCGAGTCTGGCGGCCAGGACGGCGCTGGCGACGACGACGACCTCGAGGA
TCTCGAAGAAGCCGAGGAACCCGACATGGAAGAAGACGACGACCAGAAGGCCGTC
SEQ ID NO 23: hFGE leader sequence
MAAPALGLVCGRCPELGLVLLLLLLSLLCGAAG
SEQ ID NO 24; human sulfamidase mature sequence
RPRNALLLLADDGGFESGAYNNSAIATPHLDALARRSLLFRNAFTSVSSCSPSRASLLTG
LPQHQNGMYGLHQDVHEIFNSFDKVRSLPLLLSQAGVRTGIIGKKHVGPETVYPFDFAYT
EENGSVLQVGRNITRIKLLVRKFLQTQDDRPFFLYVAFHDPHRCGHSQPQYGTFCEKFG
NGESGMGRIPDWTPQAYDPLDVLVPYFVPNTPAARADLAAQYTTVGRMDQGVGLVLQ
ELRDAGVLNDTLVIFTSDNGIPFPSGRTNLYWPGTAEPLLVSSPEHPKRWGQVSEAYVSL
LDLTPTILDWFSIPYPSYAIFGSKTIHLTGRSLLPALEAEPLWATVFGSQSHHEVTMSYPM
RSVQHRHFRLVHNLNFKMPFPIDQDFYVSPTFQDLLNRTTAGQPTGWYKDLRHYYYRA
RWELYDRSRDPHETQNLATDPRFAQLLEMLRDQLAKWQWETHDPWVCAPDGVLEEKL
SPQCQPLHN
SEQ ID NO 25: coding sequence of mature sulfamidase (SGSH)
>SGSH-1 Genscript (62 bp-1501 bp, direct) 1440 bp
CGACCCCGAAACGCCCTCCTCCTCCTCGCTGATGATGGCGGTTTCGAGTCGGGTGCC
TACAACAACTCCGCTATCGCTACCCCTCACCTCGACGCTCTGGCTCGACGATCTCTG
CTCTTCCGAAACGCCTTTACCTCCGTGTCCTCTTGCTCTCCCTCGCGAGCTTCTCTGC
TCACTGGACTCCCTCAGCACCAGAACGGAATGTACGGCCTGCATCAGGACGTTCACC
ATTTCAACTCTTTTGATAAGGTCCGATCGCTCCCTCTGCTCCTGTCCCAGGCTGGTGT
TCGAACCGGTATCATTGGAAAGAAGCACGTCGGACCCGAGACCGTGTACCCTTTCG
ACTTTGCTTACACTGAGGAGAACGGCTCCGTTCTGCAGGTCGGCCGAAACATCACCC
GAATTAAGCTCCTGGTCCGAAAGTTCCTCCAGACTCAGGACGATCGACCCTTCTTTC
TGTACGTGGCCTTTCACGACCCTCACCGATGCGGACACTCTCAGCCTCAGTACGGTA
CCTTCTGTGAGAAGTTTGGAAACGGCGAGTCCGGTATGGGACGAATCCCCGACTGG
ACCCCTCAGGCTTACGACCCCCTCGATGTCCTGGTGCCTTACTTCGTTCCCAACACCC
CTGCTGCTCGAGCTGACCTCGCTGCTCAGTACACCACTGTCGGCCGAATGGATCAGG
GCGTGGGTCTCGTTCTGCAGGAGCTGCGAGACGCTGGTGTGCTCAACGATACCCTGG
TTATCTTCACTTCTGACAACGGTATTCCCTTTCCTTCGGGACGAACCAACCTGTACTG
GCCCGGAACTGCTGAGCCTCTCCTGGTCTCGTCCCCTGAGCACCCTAAGCGATGGGG
ACAGGTTTCGGAGGCTTACGTCTCCCTCCTGGACCTCACCCCCACTATCCTGGATTG
GTTCTCTATTCCCTACCCTTCGTACGCCATCTTTGGATCTAAGACCATTCATCTGACT
GGACGATCCCTCCTGCCTGCTCTCGAGGCTGAGCCTCTGTGGGCTACCGTGTTCGGC
TCCCAGTCTCACCATGAGGTTACTATGTCCTACCCCATGCGATCTGTCCAGCACCGA
CATTTCCGACTCGTGCACAACCTGAACTTCAAGATGCCCTTTCCTATCGACCAGGAT
TTCTACGTCTCTCCCACCTTTCAGGACCTCCTGAACCGAACCACTGCCGGCCAGCCT
ACCGGTTGGTACAAGGATCTCCGACACTACTACTACCGAGCTCGATGGGAGCTGTAC
GACCGATCCCGAGATCCCCATGAGACCCAGAACCTGGCCACTGACCCTCGATTCGCT
CAGCTCCTGGAGATGCTCCGAGACCAGCTGGCCAAGTGGCAGTGGGAGACCCACGA
TCCCTGGGTGTGTGCCCCCGACGGTGTGCTCGAGGAGAAGCTGTCCCCCCAGTGTCA
GCCCCTGCATAAC
SEQ ID NO 26: MNS1 anchorage domain (AA 1-163 of XP_502939.1)
MSFNIPKTTPNFSAKARKLEDQLWQASGLEKSKDSTLPLYKDKPYGEGFVARTTSGRRR
RNIIYGVVVGLLFWAIYTFSRSLDGNVSLKDGIKDYEFKGWKGRGKPKTNWVAEQNAV
KQAFVDSWNGYHKYAWGKDVYKPQTKTGKNIVIGPKPLGWFIVDSLDS
SEQ ID NO 27: Coding sequence for the MNS1 anchorage domain (AA 1-163 of
XP_502939.1)
ATGTCGTTCAACATTCCCAAGACCACCCCCAACTTCTCGGCTAAGGCTCGAAAGCTG
GAGGATCAGCTCTGGCAGGCTTCTGGACTCGAGAAGTCCAAGGACTCTACCCTGCCT
CTCTACAAGGATAAGCCCTACGGAGAGGGCTTCGTGGCTCGAACCACTTCCGGCCG
ACGACGACGAAACATCATCTACGGCGTCGTGGTTGGTCTGCTCTTCTGGGCCATCTA
CACCTTTTCTCGATCGCTGGACGGTAACGTCTCTCTCAAGGACGGAATTAAGGATTA
CGAGTTCAAGGGCTGGAAGGGTCGAGGAAAGCCCAAGACTAACTGGGTGGCCGAGC
AGAACGCTGTTAAGCAGGCCTTTGTCGACTCCTGGAACGGCTACCATAAGTACGCCT
GGGGCAAGGATGTGTACAAGCCCCAGACCAAGACTGGAAAGAACATGGGCCCCAA
GCCTCTGGGATGGTTCATCGTGGACTCTCTGGATTCC
SEQ ID NO 28: WBP1 anchorage domain (AA 400-505 of XP_502492.1)
DHLPTTGFTMLNPYYRLTLEQTGTTNFSAIYSTTFKIPDQHGVFTFNLDYKRPGYTFIEEK
TRATIRHTANDEWPRSWEITNSWVYLTSAVMVVIAWFLFVVFYLFVGKADKEAVHKQ
SEQ ID NO 29: Coding sequence for the WBP1 anchorage domain (AA 400-505 of
XP_502492.1)
GATCACCTCCCCACCACTGGCTTCACCATGCTGAACCCCTACTACCGACTGACCCTC
GAGCAGACTGGCACCACTAACTTCTCCGCCATCTACTCTACCACTTTTAAGATTCCT
GACCAGCATGGCGTGTTCACCTTTAACCTCGATTACAAGCGACCCGGTTACACCTTC
ATCGAGGAGAAGACCCGAGCCACTATTCGACACACCGCTAACGACGAGTGGCCCCG
ATCCTGGGAGATCACCAACTCTTGGGTCTACCTGACTTCGGCCGTGATGGTCGTGAT
TGCTTGGTTCCTCTTCGTGGTGTTCTACCTGTTTGTGGGAAAGGCTGATAAGGAAGCT
GTTCATAAGCAG
SEQ ID NO 30: ERp44 mature protein
EITSLDTENIDEILNNADVALVNFYADWCRFSQMLHPIFEEASDVIKEEFPNENQVVFAR
VDCDQHSDIAQRYRISKYPTLKLFRNGMMMKREYRGQRSVKALADYIRQQKSDPIQEIR
DLAEITTLDRSKRNIIGYFEQKDSDNYRVFERVANILHDDCAFLSAFGDVSKPERYSGDN
ITYKPPGHSAPDMVYLGAMTNFDVTYNWIQDKCVPLVREITFENGEELTEEGLPFLILFH
MKEDTESLEIFQNEVARQLISEKGTINFLHADCDKFRHPLLHIQKTPADCPVIAIDSFRHM
YVFGDFKDVLIPGKLKQFVFDLHSGKLHREFHHGPDPTDTAPGEQAQDVASSPPESSFQ
KLAPSEYRYTLLRD
SEQ ID NO 31: Coding sequence for the ERp44 mature protein
GAGATTACTTCCCTGGATACTGAGAACATCGACGAGATTCTGAACAACGCCGACGT
GGCCCTGGTCAACTTCTACGCCGACTGGTGCCGATTTTCCCAGATGCTCCACCCCAT
CTTCGAGGAGGCTTCTGATGTGATTAAGGAGGAGTTCCCTAACGAGAACCAGGTCGT
GTTTGCCCGAGTTGACTGTGATCAGCATTCTGACATCGCTCAGCGATACCGAATTTC
GAAGTACCCCACCCTGAAGCTCTTCCGAAACGGAATGATGATGAAGCGAGAGTACC
GAGGCCAGCGATCGGTTAAGGCCCTGGCTGACTACATCCGACAGCAGAAGTCCGAC
CCCATCCAGGAGATTCGAGATCTGGCCGAGATTACCACTCTCGACCGATCTAAGCGA
AACATCATTGGTTACTTCGAGCAGAAGGACTCGGATAACTACCGAGTGTTTGAGCGA
GTTGCTAACATCCTGCACGACGATTGCGCCTTCCTCTCTGCTTTTGGAGACGTCTCGA
AGCCCGAGCGATACTCCGGCGACAACATCATCTACAAGCCCCCTGGACATTCTGCCC
CTGACATGGTTTACCTGGGCGCTATGACCAACTTCGACGTCACTTACAACTGGATTC
AGGATAAGTGTGTTCCCCTCGTCCGAGAGATTACCTTTGAGAACGGCGAGGAGCTG
ACTGAGGAGGGTCTCCCTTTCCTGATCCTCTTTCACATGAAGGAGGATACCGAGTCC
CTGGAGATTTTCCAGAACGAGGTGGCCCGACAGCTGATCTCCGAGAAGGGAACTAT
TAACTTCCTCCACGCTGACTGCGATAAGTTTCGACACCCCCTGCTCCATATCCAGAA
GACCCCCGCCGACTGTCCTGTCATCGCTATTGATTCTTTCCGACACATGTACGTCTTC
GGCGACTTTAAGGATGTGCTGATTCCCGGCAAGCTGAAGCAGTTCGTGTTTGACCTG
CACTCCGGAAAGCTCCATCGAGAGTTCCACCATGGCCCCGACCCTACCGATACTGCC
CCTGGAGAGCAGGCCCAGGACGTTGCTTCCTCTCCCCCTGAGTCGTCCTTCCAGAAG
CTGGCCCCCTCCGAGTACCGATACACCCTCCTGCGAGAC
SEQ ID NO 32: Fusion construct: LIP2-BtFGE-6xHis-HDEL
MKLSTILFTACATLAAAAGGEEAGPEAGAPSLVGSCGCGNPQRPGAQGSSAAAHRYSR
EANAPGSVPGGRPSPPTKMVPIPAGVFTMGTDDPQIKQDGEAPARRVAIDAFYMDAYEV
SNAEFEKFVNSTGYLTEAEKFGDSFVFEGMLSEQVKSDIQQAVAAAPWWLPVKGANW
RHPEGPDSTVLHRPDHPVLHVSWNDAVAYCTWAGKRLPTEAEWEYSCRGGLQNRLFP
WGNKLQPKGQHYANIWQGEFPVTNTGEDGFRGTAPVDAFPPNGYGLYNIVGNAWEWT
SDWWTVHHSAEETINPKGPPSGKDRVKKGGSYMCHKSYCYRYRCAARSQNTPDSSAS
NLGFRCAADHLPTTGADHLPTTGHHHHHHHDEL
SEQ ID NO 33: RDEL
RDEL
SEQ ID NO 34: Conserved sequence of Iduronate Sulfatase
CAPSRVSFLTGR
SEQ ID NO 35: MNS1-HpFGE-6xHis fusion construct
MSFNIPKTTPNFSAKARKLEDQLWQASGLEKSKDSTLPLYKDKPYGEGFVARTTSGRRR
RNIIYGVVVGLLFWAIYTFSRSLDGNVSLKDGIKDYEFKGWKGRGKPKTNWVAEQNAV
KQAFVDSWNGYHKYAWGKDVYKPQTKTGKNMGPKPLGWFIVDSLDSMGTDKAKIYL
DGESPSRLVTLDPYYFDVYEVSNSEFELFVNTTSYITEAEKFGDSFVLEARISEEVKKDIS
QVVAAAPWWLPVKGAEWRHPEGPDSSISSRMDHPVTHISWNDATAYCQWAGKRLPTE
AEWENAARGGLNNRLFPWGNKLMPKDHHRVNIWQGEFPKVNTAEDGYEGTCPVTAFE
PNGYGLYNTVGNAWEWVADWWTTVHSPESQNNPVGPDEGTDKVKKGGSYMCHISYC
YRYRCEARSQNSPDSSACNLGFRCAATNLPEDIPCSNCNDSTPHHHHHH
SEQ ID NO 36: Coding sequence for MNS1-HpFGE-6xHis fusion construct
ATGTCGTTCAACATTCCCAAGACTACCCCTAACTTCTCGGCTAAGGCTCGAAAGCTG
GAGGATCAGCTCTGGCAGGCTTCTGGACTGGAGAAGTCCAAGGACTCTACCCTGCC
CCTCTACAAGGATAAGCCTTACGGAGAGGGATTCGTGGCTCGAACCACCTCCGGCC
GACGACGACGAAACATCATCTACGGCGTCGTGGTTGGTCTGCTCTTCTGGGCTATCT
ACACCTTTTCCCGATCTCTGGACGGCAACGTCTCCCTCAAGGACGGTATTAAGGATT
ACGAGTTCAAGGGATGGAAGGGCCGAGGCAAGCCCAAGACCAACTGGGTGGCTGA
GCAGAACGCCGTGAAGCAGGCTTTTGTTGACTCTTGGAACGGATACCACAAGTACG
CCTGGGGCAAGGATGTCTACAAGCCCCAGACCAAGACTGGAAAGAACATGGGCCCC
AAGCCTCTGGGCTGGTTCATCGTGGACTCGCTCGATTCCATGGGCACCGACAAGGCC
AAGATCTACCTGGATGGTGAGTCGCCCTCCCGACTGGTTACTCTCGACCCTTACTAC
TTTGATGTTTACGAGGTCTCTAACTCGGAGTTCGAGCTGTTTGTCAACACCACTTCTT
ACATCACCGAGGCCGAGAAGTTCGGTGACTCCTTTGTCCTCGAGGCTCGAATCTCTG
AGGAAGTCAAGAAGGATATTTCTCAGGTGGTGGCCGCTGCCCCCTGGTGGCTCCCTG
TTAAGGGTGCTGAGTGGCGACACCCTGAGGGACCTGACTCCTCTATCTCGTCCCGAA
TGGATCACCCCGTTACCCATATTTCCTGGAACGACGCTACTGCCTACTGTCAGTGGG
CTGGCAAGCGACTGCCTACCGAGGCTGAGTGGGAGAACGCTGCTCGAGGCGGTCTG
AACAACCGACTCTTCCCCTGGGGAAACAAGCTCATGCCTAAGGACCACCATCGAGT
GAACATCTGGCAGGGCGAGTTCCCCAAGGTTAACACCGCCGAGGACGGTTACGAGG
GAACCTGCCCCGTGACTGCTTTTGAGCCTAACGGATACGGCCTGTACAACACTGTCG
GAAACGCCTGGGAGTGGGTGGCTGACTGGTGGACCACTGTTCACTCTCCCGAGTCGC
AGAACAACCCCGTTGGTCCTGACGAGGGAACCGATAAGGTCAAGAAGGGAGGCTCG
TACATGTGCCATATTTCTTACTGTTACCGATACCGATGCGAGGCCCGATCCCAGAAC
TCTCCCGACTCTTCGGCTTGTAACCTGGGTTTCCGATGCGCTGCCACCAACCTCCCTG
AGGACATTCCCTGCTCTAACTGTAACGACTCCACTCCCCACCACCATCACCATCACT
AA
SEQ ID NO 37: MNS1-BtFGE-6xHis fusion construct
MSFNIPKTTPNFSAKARKLEDQLWQASGLEKSKDSTLPLYKDKPYGEGFVARTTSGRRR
RNIIYGVVVGLLFWAIYTFSRSLDGNVSLKDGIKDYEFKGWKGRGKPKTNWVAEQNAV
KQAFVDSWNGYHKYAWGKDVYKPQTKTGKNIVIGPKPLGWFIVDSLDSGGEEAGPEAG
APSLVGSCGCGNPQRPGAQGSSAAAHRYSREANAPGSVPGGRPSPPTKMVPIPAGVFTM
GTDDPQIKQDGEAPARRVAIDAFYIVIDAYEVSNAEFEKFVNSTGYLTEAEKFGDSFVFEG
MLSEQVKSDIQQAVAAAPWWLPVKGANWRHPEGPDSTVLHRPDHPVLHVSWNDAVA
YCTWAGKRLPTEAEWEYSCRGGLQNRLFPWGNKLQPKGQHYANIWQGEFPVTNTGED
GFRGTAPVDAFPPNGYGLYNIVGNAWEWTSDWWTVHHSAEETINPKGPPSGKDRVKK
GGSYMCHKSYCYRYRCAARSQNTPDSSASNLGFRCAADHLPTTGADHLPTTGHHHHH
H
SEQ ID NO 38: Coding sequence for the MNS1-BtFGE-6xHis fusion construct
ATGTCGTTCAACATTCCCAAGACCACCCCCAACTTCTCGGCTAAGGCTCGAAAGCTG
GAGGATCAGCTCTGGCAGGCTTCTGGACTCGAGAAGTCCAAGGACTCTACCCTGCCT
CTCTACAAGGATAAGCCCTACGGAGAGGGCTTCGTGGCTCGAACCACTTCCGGCCG
ACGACGACGAAACATCATCTACGGCGTCGTGGTTGGTCTGCTCTTCTGGGCCATCTA
CACCTTTTCTCGATCGCTGGACGGTAACGTCTCTCTCAAGGACGGAATTAAGGATTA
CGAGTTCAAGGGCTGGAAGGGTCGAGGAAAGCCCAAGACTAACTGGGTGGCCGAGC
AGAACGCTGTTAAGCAGGCCTTTGTCGACTCCTGGAACGGCTACCATAAGTACGCCT
GGGGCAAGGATGTGTACAAGCCCCAGACCAAGACTGGAAAGAACATGGGCCCCAA
GCCTCTGGGATGGTTCATCGTGGACTCTCTGGATTCCGGCGGCGAGGAAGCCGGTCC
TGAGGCTGGAGCTCCTTCTCTGGTTGGCTCGTGCGGCTGTGGAAACCCCCAGCGACC
TGGTGCTCAGGGCTCCTCTGCCGCTGCCCACCGATACTCTCGAGAGGCCAACGCTCC
CGGTTCTGTGCCTGGAGGCCGACCTTCGCCCCCTACCAAGATGGTGCCCATTCCTGC
TGGAGTTTTCACCATGGGCACTGACGATCCTCAGATCAAGCAGGACGGAGAGGCTC
CTGCTCGACGAGTTGCCATTGACGCTTTTTACATGGATGCTTACGAGGTTTCTAACGC
CGAGTTCGAGAAGTTTGTCAACTCGACCGGATACCTGACTGAGGCCGAGAAGTTCG
GAGACTCCTTCGTCTTTGAGGGCATGCTCTCCGAGCAGGTCAAGTCTGACATCCAGC
AGGCTGTGGCTGCCGCTCCTTGGTGGCTGCCCGTTAAGGGTGCTAACTGGCGACATC
CTGAGGGTCCTGACTCCACCGTCCTGCACCGACCCGATCATCCTGTCCTCCACGTGT
CTTGGAACGACGCCGTGGCTTACTGTACCTGGGCTGGCAAGCGACTGCCTACTGAGG
CTGAGTGGGAGTACTCTTGCCGAGGTGGACTGCAGAACCGACTCTTCCCTTGGGGTA
ACAAGCTCCAGCCCAAGGGACAGCACTACGCCAACATTTGGCAGGGCGAGTTTCCT
GTCACCAACACTGGCGAGGACGGTTTCCGAGGAACCGCTCCCGTGGATGCCTTTCCC
CCTAACGGATACGGCCTGTACAACATCGTGGGTAACGCTTGGGAGTGGACCTCCGA
CTGGTGGACTGTTCACCATTCTGCCGAGGAGACCATTAACCCTAAGGGCCCTCCCTC
TGGCAAGGACCGAGTCAAGAAGGGCGGTTCGTACATGTGCCACAAGTCCTACTGTT
ACCGATACCGATGCGCCGCTCGATCGCAGAACACCCCTGACTCTTCTGCTTCCAACC
TCGGCTTCCGATGTGCCGCTGATCACCTCCCCACCACTGGCGCTGACCACCTGCCCA
CTACTGGACACCACCACCACCACCATTAA
SEQ ID NO 39: Lip2pre-6xHis-BtFGE-WBP1fusion construct
MKLSTILFTACATLAAAHHHHHHAGGEEAGPEAGAPSLVGSCGCGNPQRPGAQGSSAA
AHRYSREANAPGSVPGGRPSPPTKMVPIPAGVFTMGTDDPQIKQDGEAPARRVAIDAFY
MDAYEVSNAEFEKFVNSTGYLTEAEKFGDSFVFEGMLSEQVKSDIQQAVAAAPWWLPV
KGANWRHPEGPDSTVLHRPDHPVLHVSWNDAVAYCTWAGKRLPTEAEWEYSCRGGL
QNRLFPWGNKLQPKGQHYANIWQGEFPVTNTGEDGFRGTAPVDAFPPNGYGLYNIVGN
AWEWTSDWWTVHHSAEETINPKGPPSGKDRVKKGGSYMCHKSYCYRYRCAARSQNT
PDSSASNLGFRCAADHLPTTGADHLPTTGFTMLNPYYRLTLEQTGTTNFSAIYSTTFKIPD
QHGVFTFNLDYKRPGYTFIEEKTRATIRHTANDEWPRSWEITNSWVYLTSAVMVVIAWF
LFVVFYLFVGKADKEAVHKQ
SEQ ID NO 40: Coding sequence for the Lip2pre-6xHis-BtFGE-WBP1fusion construct
ATGAAGCTGTCTACCATTCTGTTTACTGCTTGTGCTACCCTGGCTGCTGCCCACCACC
ATCACCATCACGCTGGCGGAGAAGAGGCTGGACCCGAGGCTGGAGCTCCTTCCCTG
GTGGGATCGTGTGGATGTGGAAACCCTCAGCGACCTGGAGCTCAGGGTTCTTCTGCC
GCTGCCCATCGATACTCCCGAGAGGCTAACGCTCCTGGTTCTGTGCCTGGCGGACGA
CCTTCTCCTCCCACCAAGATGGTCCCCATCCCTGCCGGAGTTTTCACCATGGGTACTG
ACGATCCTCAGATCAAGCAGGACGGAGAGGCTCCTGCTCGACGAGTTGCCATTGAC
GCTTTTTACATGGATGCCTACGAGGTCTCTAACGCTGAGTTCGAGAAGTTTGTTAAC
TCCACCGGATACCTCACTGAGGCCGAGAAGTTCGGCGACTCCTTCGTCTTTGAGGGA
ATGCTGTCGGAGCAGGTTAAGTCTGATATTCAGCAGGCTGTGGCTGCCGCTCCTTGG
TGGCTGCCCGTCAAGGGAGCTAACTGGCGACATCCCGAGGGTCCTGACTCGACCGTT
CTGCACCGACCCGATCATCCTGTTCTCCACGTGTCTTGGAACGACGCTGTGGCTTAC
TGCACCTGGGCTGGAAAGCGACTCCCCACTGAGGCTGAGTGGGAGTACTCTTGTCGA
GGTGGCCTGCAGAACCGACTCTTCCCTTGGGGTAACAAGCTGCAGCCCAAGGGCCA
GCACTACGCCAACATCTGGCAGGGAGAGTTTCCTGTTACCAACACTGGAGAGGACG
GATTCCGAGGTACCGCTCCTGTGGATGCTTTTCCCCCTAACGGTTACGGCCTCTACA
ACATCGTGGGCAACGCCTGGGAGTGGACCTCGGACTGGTGGACTGTCCACCATTCTG
CTGAGGAGACCATTAACCCCAAGGGTCCCCCTTCTGGCAAGGATCGAGTGAAGAAG
GGAGGTTCCTACATGTGTCACAAGTCGTACTGCTACCGATACCGATGTGCCGCTCGA
TCCCAGAACACCCCTGACTCGTCTGCCTCGAACCTGGGATTCCGATGCGCCGCTGAC
CATCTGCCTACCACTGGCGCTGATCACCTCCCCACCACTGGCTTCACCATGCTGAAC
CCCTACTACCGACTGACCCTCGAGCAGACTGGCACCACTAACTTCTCTGCCATCTAC
TCCACCACTTTTAAGATTCCTGACCAGCATGGTGTCTTCACCTTTAACCTCGATTACA
AGCGACCCGGCTACACTTTCATCGAGGAGAAGACCCGAGCCACTATTCGACACACC
GCTAACGACGAGTGGCCCCGATCTTGGGAGATCACCAACTCCTGGGTGTACCTGACT
TCGGCCGTCATGGTGGTCATTGCTTGGTTCCTGTTCGTCGTGTTTTACCTGTTCGTTG
GCAAGGCTGACAAGGAAGCTGTTCATAAGCAGTAA
SEQ ID NO 41: Chimeric Lip2pre-BtFGE-HpFGE-6xHis-HDEL fusion construct
MKLSTILFTACATLAAAAGGEEAGPEAGAPSLVGSCGCGNPQRPGAQGSSAAAHRYSR
EANAPGSVPGGRPSPPTKMVPIPAGVFTMGTDKAKIYLDGESPSRLVTLDPYYFDVYEV
SNSEFELFVNTTSYITEAEKFGDSFVLEARISEEVKKDISQVVAAAPWWLPVKGAEWRH
PEGPDSSISSRMDHPVTHISWNDATAYCQWAGKRLPTEAEWENAARGGLNNRLFPWGN
KLMPKDHHRVNIWQGEFPKVNTAEDGYEGTCPVTAFEPNGYGLYNTVGNAWEWVAD
WWTTVHSPESQNNPVGPDEGTDKVKKGGSYMCHISYCYRYRCEARSQNSPDSSACNLG
FRCAATNLPEDIPCSNCNDSTPHHHHHHHDEL
SEQ ID NO 42: Coding sequence for the Chimeric Lip2pre-BtFGE-HpFGE-6xHis-HDEL fusion
construct
ATGAAGCTGTCTACTATTCTGTTTACTGCTTGCGCTACTCTGGCTGCCGCTGCCGGAG
GCGAGGAAGCTGGTCCCGAGGCTGGTGCTCCCTCTCTGGTGGGTTCGTGCGGCTGTG
GAAACCCCCAGCGACCTGGTGCTCAGGGCTCCTCTGCCGCTGCCCACCGATACTCTC
GAGAGGCTAACGCTCCTGGATCGGTCCCTGGCGGTCGACCCTCTCCCCCTACCAAGA
TGGTGCCCATCCCTGCCGGTGTTTTCACCATGGGAACTGACAAGGCTAAGATCTACC
TGGATGGCGAGTCGCCTTCCCGACTGGTCACCCTCGACCCCTACTACTTTGATGTTTA
CGAGGTCTCTAACTCGGAGTTCGAGCTGTTTGTGAACACCACTTCTTACATCACTGA
GGCCGAGAAGTTCGGTGACTCCTTTGTCCTCGAGGCTCGAATCTCTGAGGAAGTCAA
GAAGGATATTTCTCAGGTGGTGGCTGCCGCTCCTTGGTGGCTCCCCGTTAAGGGTGC
TGAGTGGCGACACCCTGAGGGTCCTGACTCGTCCATCTCTTCGCGAATGGATCACCC
TGTCACCCATATTTCCTGGAACGACGCCACTGCTTACTGTCAGTGGGCTGGCAAGCG
ACTGCCCACCGAGGCTGAGTGGGAGAACGCTGCTCGAGGCGGCCTGAACAACCGAC
TCTTCCCTTGGGGAAACAAGCTCATGCCCAAGGACCACCATCGAGTGAACATTTGGC
AGGGCGAGTTCCCCAAGGTTAACACCGCTGAGGACGGATACGAGGGTACCTGCCCT
GTGACTGCTTTTGAGCCCAACGGATACGGCCTCTACAACACTGTCGGAAACGCCTGG
GAGTGGGTGGCTGACTGGTGGACCACTGTTCACTCCCCCGAGTCTCAGAACAACCCC
GTTGGACCTGACGAGGGCACCGATAAGGTCAAGAAGGGCGGCTCCTACATGTGCCA
TATCTCTTACTGTTACCGATACCGATGCGAGGCCCGATCGCAGAACTCCCCTGACTC
CTCTGCTTGTAACCTGGGTTTCCGATGCGCCGCTACCAACCTCCCCGAGGATATTCC
CTGTTCCAACTGTAACGATTCCACCCCTCACCACCATCACCATCATCACGACGAGCT
GTAA
SEQ ID NO 43: Tupaia chinensis FGE
EEARTGAGATSAQGPCGCGTPQRPGSHGSSAAAHRYSREANVPGPVPGERQPEATKMV
PIPAGVFTMGTDDPQIKQDGEAPARRVAIDAFYMDAYEVSNAEFEKFVNSTGYLTEAEK
FGDSFVFEGMLSEQVKTGIQQAVAAAPWWLPVKGANWRHPEGPDSTILHRADHPVLH
VSWNDAVAYCTWAGKRLPTEAEWEYSCRGGLQNRLFPWGNKLQPRGQHYANIWQGE
FPVTNTAEDGFQGTAPVDAFPPNGYGLYNIVGNAWEWTSDWWTVYHSVEETLNPKGP
PSGKDRVKKGGSYMCHKSYCYRYRCAARSQNTPDSSASNLGFRCAADRLPT
SEQ ID NO 44: Coding sequence for the Tupaia chinensis FGE
GAGGAAGCCCGAACTGGTGCTGGTGCTACTTCTGCTCAGGGACCCTGCGGTTGCGGT
ACTCCTCAGCGACCCGGTTCTCACGGCTCGTCTGCCGCTGCCCACCGATACTCTCGA
GAGGCTAACGTTCCTGGACCTGTCCCCGGAGAGCGACAGCCTGAGGCCACCAAGAT
GGTCCCTATCCCCGCTGGCGTGTTCACCATGGGTACTGACGATCCTCAGATCAAGCA
GGACGGTGAAGCTCCTGCTCGACGAGTTGCCATTGACGCTTTTTACATGGATGCCTA
CGAGGTGTCCAACGCTGAGTTCGAGAAGTTTGTTAACTCTACCGGATACCTGACTGA
GGCCGAGAAGTTCGGAGACTCCTTCGTCTTTGAGGGCATGCTCTCTGAGCAGGTTAA
GACCGGCATCCAGCAGGCTGTGGCTGCCGCTCCTTGGTGGCTGCCTGTGAAGGGAG
CTAACTGGCGACATCCTGAGGGTCCCGACTCCACTATTCTGCACCGAGCTGATCATC
CTGTCCTCCACGTGTCTTGGAACGACGCCGTCGCTTACTGTACCTGGGCTGGCAAGC
GACTGCCTACTGAGGCTGAGTGGGAGTACTCCTGCCGAGGCGGTCTGCAGAACCGA
CTCTTCCCTTGGGGTAACAAGCTCCAGCCCCGAGGACAGCACTACGCCAACATCTGG
CAGGGAGAGTTTCCTGTCACCAACACTGCTGAGGACGGATTCCAGGGCACCGCTCCT
GTGGATGCTTTTCCCCCTAACGGTTACGGACTGTACAACATTGTTGGAAACGCCTGG
GAGTGGACCTCGGACTGGTGGACTGTGTACCATTCCGTTGAGGAGACCCTCAACCCC
AAGGGTCCCCCTTCTGGAAAGGATCGAGTGAAGAAGGGAGGCTCGTACATGTGCCA
CAAGTCCTACTGTTACCGATACCGATGCGCCGCTCGATCTCAGAACACCCCCGACTC
CTCTGCCTCGAACCTCGGATTCCGATGTGCTGCTGACCGACTGCCCACT
SEQ ID NO 45: Monodelphis domestica FGE
AARGLGSEAGSAAADAAHPAGTCGCGSPQRPGTAAHRYSREANVAEPASAERPVLTSQ
MAHIPAGVFTMGTDEPQIKQDGEGPARRVRINSFYMDLYEVSNAEFERFVNSTGYVTEA
EKFGDSFVFDSMLSDQVKSDIHQAVAAAPWWLPVKGANWRHPEGPDSSILHRRDHPVL
HVSWNDAVAYCTWAGKRLPTEAEWEYSCRGGLENRLFPWGNKLQPKGQHYANIWQG
EFPVSNTGEDGYQGTAPVTAFPPNGYGLYNIVGNAWEWTSDWWTVHHSADETLDPKG
PPSGSDRVKKGGSYMCHKSYCYRYRCAARSQNTPDSSASNLGFRCAADRLPDT
SEQ ID NO 46: Coding sequence for the Monodelphis domestica FGE
GCCGCCCGAGGTCTGGGTTCCGAGGCCGGTTCCGCCGCCGCCGACGCCGCTCACCCT
GCTGGCACTTGTGGTTGTGGTTCCCCTCAGCGACCCGGCACCGCCGCTCACCGATAC
TCTCGAGAGGCTAACGTGGCTGAGCCTGCTTCTGCCGAGCGACCTGTGCTGACTTCG
CAGATGGCTCACATCCCCGCCGGTGTCTTCACCATGGGAACTGACGAGCCCCAGATC
AAGCAGGATGGAGAGGGACCTGCCCGACGAGTTCGAATTAACTCGTTTTACATGGA
CCTCTACGAGGTCTCCAACGCTGAGTTCGAGCGATTTGTTAACTCCACCGGTTACGT
CACTGAGGCCGAGAAGTTCGGAGACTCTTTCGTTTTTGATTCCATGCTGTCTGACCA
GGTGAAGTCCGATATCCATCAGGCTGTGGCCGCTGCCCCCTGGTGGCTCCCTGTCAA
GGGAGCTAACTGGCGACACCCTGAGGGACCTGACTCCTCTATTCTGCACCGACGAG
ATCATCCCGTCCTCCACGTGTCTTGGAACGACGCTGTGGCCTACTGTACCTGGGCTG
GAAAGCGACTGCCTACTGAGGCTGAGTGGGAGTACTCCTGCCGAGGCGGTCTGGAG
AACCGACTCTTTCCCTGGGGCAACAAGCTCCAGCCTAAGGGTCAGCACTACGCTAAC
ATCTGGCAGGGCGAGTTCCCCGTCTCCAACACCGGAGAGGACGGCTACCAGGGCAC
CGCTCCTGTGACTGCCTTTCCCCCTAACGGCTACGGTCTGTACAACATTGTGGGTAA
CGCTTGGGAGTGGACCTCCGACTGGTGGACTGTTCACCATTCTGCCGACGAGACCCT
CGATCCCAAGGGACCCCCTTCTGGCTCGGATCGAGTTAAGAAGGGAGGCTCGTACA
TGTGCCACAAGTCCTACTGTTACCGATACCGATGCGCTGCCCGATCTCAGAACACCC
CTGACTCTTCCGCCTCTAACCTGGGCTTCCGATGTGCTGCTGACCGACTGCCTGACA
CT
SEQ ID NO 47: Gallus gallus FGE
GKETAPGGNCGCSASRSRGGEREAVATVRRYSAAANDGRSSGRGPMVAIPGGVFTMGT
DEPEIQQDGEWPARRVHVNSFYMDQYEVSNQEFERFVNSTGYLTEAEKFGDSFVFEGM
LSEEVKAEIHQAVAAAPWWLPVKGANWRQPEGPGSSILSRMDHPVLHVSWNDAVAFC
TWAGKRLPTEAEWEYGCRGGLEKRLFPWGNKLQPKGQHYANIWQGVFPTNNTAEDGY
KGTAPVTAFPPNGYGLYNIVGNAWEWTSDWWAVHHSADEAHNPKGPSSGTDRVKKG
GSYMCHKSYCYRYRCAARSQNTPDSSASNLGFRCAADALPDPQ
SEQ ID NO 48: Coding sequence for the Gallus gallus FGE
GGCAAGGAGACTGCCCCTGGCGGTAACTGCGGTTGTTCTGCTTCCCGATCCCGAGGT
GGAGAGCGAGAGGCCGTTGCTACTGTCCGACGATACTCCGCCGCTGCCAACGACGG
CCGATCCTCTGGCCGAGGTCCCATGGTGGCTATCCCTGGCGGTGTTTTCACCATGGG
AACTGACGAGCCCGAGATTCAGCAGGATGGCGAGTGGCCTGCTCGACGAGTCCACG
TGAACTCGTTTTACATGGACCAGTACGAGGTTTCTAACCAGGAGTTCGAGCGATTTG
TCAACTCTACCGGATACCTGACTGAGGCCGAGAAGTTCGGCGACTCTTTCGTTTTTG
AGGGAATGCTCTCGGAGGAAGTCAAGGCCGAGATCCATCAGGCTGTTGCTGCCGCT
CCTTGGTGGCTGCCTGTGAAGGGTGCTAACTGGCGACAGCCTGAGGGACCTGGCTCG
TCCATTCTGTCCCGAATGGACCACCCCGTTCTCCATGTCTCTTGGAACGATGCCGTCG
CTTTCTGTACCTGGGCTGGCAAGCGACTGCCTACTGAGGCTGAGTGGGAGTACGGAT
GCCGAGGCGGCCTGGAGAAGCGACTCTTTCCCTGGGGCAACAAGCTCCAGCCTAAG
GGTCAGCACTACGCCAACATCTGGCAGGGCGTCTTCCCCACCAACAACACTGCTGA
GGACGGCTACAAGGGCACCGCCCCTGTGACTGCTTTTCCCCCTAACGGTTACGGACT
GTACAACATTGTGGGTAACGCCTGGGAGTGGACCTCTGACTGGTGGGCTGTTCACCA
TTCTGCCGATGAGGCTCACAACCCCAAGGGACCTTCTTCGGGCACCGACCGAGTGA
AGAAGGGTGGATCGTACATGTGCCATAAGTCCTACTGTTACCGATACCGATGCGCCG
CTCGATCCCAGAACACCCCCGATTCCTCTGCCTCTAACCTCGGTTTCCGATGTGCCGC
CGACGCCCTCCCCGACCCTCAG
SEQ ID NO 49: Dendroctonus ponderosa FGE
ICDCGCSLNRDGQCNSEDNEINPSQKYKRDLNENPADNFDKSQMALIGKGIFEMGTNKP
VFPSDFEGPARNVTIENSFYLDLYEVSNQQFYDFVRTTNYKTEAEQFGDSFVFEMSLPEN
QRNEHQDIRAAQAPWWIKLPDAYWKHPEGPKSTIEDRIVINHPVAHVSWNDAVAYCEYV
GKRLPTEAEWEMACRGGLRQKMYPWGNKLQPKGQHWANIWQGEFPKENTAEDGYIF
TCPVDKFPPNQFGLYNMAGNVWEWVQDDWQTDPQNSRVKKGGSFLCHQSYCWRYRC
AARSFNTKDSSAANLGFRCAADAR
SEQ ID NO 50: Coding sequence for the Dendroctonus ponderosa FGE
ATTTGCGACTGCGGCTGCTCCCTGAACCGAGACGGCCAGTGTAACTCCGAGGACAA
CGAGATTAACCCCTCCCAGAAGTACAAGCGAGACCTGAACGAGAACCCCGCCGACA
ACTTCGATAAGTCTCAGATGGCTCTCATCGGCAAGGGAATTTTTGAGATGGGCACCA
ACAAGCCCGTTTTCCCTTCGGACTTTGAGGGTCCTGCCCGAAACGTCACTATCGAGA
ACTCCTTCTACCTGGACCTCTACGAGGTCTCTAACCAGCAGTTCTACGATTTTGTGCG
AACCACTAACTACAAGACCGAGGCTGAGCAGTTCGGTGACTCGTTCGTCTTTGAGAT
GTCCCTGCCCGAGAACCAGCGAAACGAGCACCAGGACATCCGAGCTGCTCAGGCTC
CTTGGTGGATTAAGCTCCCTGATGCTTACTGGAAGCATCCCGAGGGACCTAAGTCGA
CCATTGAGGACCGAATGAACCACCCCGTCGCCCATGTGTCCTGGAACGATGCCGTG
GCTTACTGTGAGTACGTTGGCAAGCGACTGCCTACTGAGGCTGAGTGGGAGATGGCT
TGCCGAGGCGGTCTGCGACAGAAGATGTACCCCTGGGGAAACAAGCTCCAGCCTAA
GGGCCAGCACTGGGCCAACATCTGGCAGGGAGAGTTCCCCAAGGAGAACACCGCTG
AGGACGGATACATTTTTACTTGTCCTGTGGATAAGTTCCCTCCCAACCAGTTTGGCCT
CTACAACATGGCCGGTAACGTTTGGGAGTGGGTCCAGGACGATTGGCAGACCGACC
CCCAGAACTCCCGAGTTAAGAAGGGAGGCTCTTTCCTGTGCCATCAGTCGTACTGTT
GGCGATACCGATGCGCCGCTCGATCTTTCAACACCAAGGACTCCTCTGCCGCTAACC
TCGGATTCCGATGTGCTGCTGACGCCCGA
SEQ ID NO 51: Columba livia FGE
MVVIPGGVFTMGTDEPAIQQDGEWPVRKVHVNSFYMDRYEVSNEDFERFVNSTGYVTE
AEKFGDSFVFEGMLSEEVKAEIHQAVAAAPWWLPVKGANWKHPEGPDSNISNRMDHP
VLHVSWNDAVAFCTWAGKRLPTEAEWEYSCRGGLENRLFPWGNKLQPKGQHYANIW
QGVFPTNNTAEDGYKGTAPVTAFPPNGYGLYNIVGNAWEWTADWWAVHHSTEEVHN
PKGPSSGTDRVKKGGSYMCHKSYCYRYRCAARSQNTPDSSASNLGFRCAADASPELP
SEQ ID NO 52: Coding sequence for the Columba livia FGE
ATGGTCGTTATTCCCGGAGGAGTTTTTACTATGGGTACTGATGAGCCCGCTATCCAG
CAGGACGGAGAGTGGCCCGTGCGAAAGGTTCACGTTAACTCTTTCTACATGGACCG
ATACGAGGTCTCGAACGAGGATTTCGAGCGATTTGTTAACTCCACCGGCTACGTCAC
TGAGGCTGAGAAGTTTGGTGACTCGTTCGTCTTTGAGGGAATGCTGTCCGAGGAAGT
CAAGGCTGAGATCCACCAGGCTGTGGCCGCTGCCCCCTGGTGGCTCCCTGTGAAGG
GAGCTAACTGGAAGCATCCCGAGGGCCCTGACTCTAACATTTCGAACCGAATGGAT
CACCCCGTCCTGCATGTGTCCTGGAACGATGCTGTTGCCTTCTGTACCTGGGCTGGC
AAGCGACTGCCTACTGAGGCCGAGTGGGAGTACTCTTGCCGAGGCGGTCTGGAGAA
CCGACTCTTTCCCTGGGGCAACAAGCTGCAGCCTAAGGGTCAGCACTACGCTAACAT
CTGGCAGGGTGTGTTCCCCACCAACAACACTGCCGAGGACGGCTACAAGGGCACCG
CTCCTGTGACTGCCTTTCCCCCTAACGGTTACGGACTCTACAACATTGTTGGAAACG
CTTGGGAGTGGACCGCTGACTGGTGGGCTGTGCACCATTCTACTGAGGAAGTCCACA
ACCCCAAGGGACCTTCCTCTGGCACCGATCGAGTCAAGAAGGGAGGCTCCTACATG
TGCCATAAGTCTTACTGTTACCGATACCGATGCGCTGCCCGATCCCAGAACACCCCC
GACTCGTCCGCCTCTAACCTGGGATTCCGATGTGCTGCCGACGCTTCGCCTGAGCTG
CCC
SEQ ID NO 53: Tupaia chinensis Lip2-TupFGE-His6-HDEL fusion construct
MKLSTILFTACATLAAAEEARTGAGATSAQGPCGCGTPQRPGSHGSSAAAHRYSREAN
VPGPVPGERQPEATKMVPIPAGVFTMGTDDPQIKQDGEAPARRVAIDAFYMDAYEVSN
AEFEKFVNSTGYLTEAEKFGDSFVFEGMLSEQVKTGIQQAVAAAPWWLPVKGANWRH
PEGPDSTILHRADHPVLHVSWNDAVAYCTWAGKRLPTEAEWEYSCRGGLQNRLFPWG
NKLQPRGQHYANIWQGEFPVTNTAEDGFQGTAPVDAFPPNGYGLYNIVGNAWEWTSD
WWTVYHSVEETLNPKGPPSGKDRVKKGGSYMCHKSYCYRYRCAARSQNTPDSSASNL
GFRCAADRLPTHHHHHHHDEL
SEQ ID NO 54: Coding sequence for the Lip2-TupFGE-His6-HDEL fusion protein
ATGAAGCTTTCCACCATCCTCTTCACAGCCTGCGCTACCCTGGCTGCCGCCGAGGAA
GCCCGAACTGGTGCTGGTGCTACTTCTGCTCAGGGACCCTGCGGTTGCGGTACTCCT
CAGCGACCCGGTTCTCACGGCTCGTCTGCCGCTGCCCACCGATACTCTCGAGAGGCT
AACGTTCCTGGACCTGTCCCCGGAGAGCGACAGCCTGAGGCCACCAAGATGGTCCC
TATCCCCGCTGGCGTGTTCACCATGGGTACTGACGATCCTCAGATCAAGCAGGACGG
TGAAGCTCCTGCTCGACGAGTTGCCATTGACGCTTTTTACATGGATGCCTACGAGGT
GTCCAACGCTGAGTTCGAGAAGTTTGTTAACTCTACCGGATACCTGACTGAGGCCGA
GAAGTTCGGAGACTCCTTCGTCTTTGAGGGCATGCTCTCTGAGCAGGTTAAGACCGG
CATCCAGCAGGCTGTGGCTGCCGCTCCTTGGTGGCTGCCTGTGAAGGGAGCTAACTG
GCGACATCCTGAGGGTCCCGACTCCACTATTCTGCACCGAGCTGATCATCCTGTCCT
CCACGTGTCTTGGAACGACGCCGTCGCTTACTGTACCTGGGCTGGCAAGCGACTGCC
TACTGAGGCTGAGTGGGAGTACTCCTGCCGAGGCGGTCTGCAGAACCGACTCTTCCC
TTGGGGTAACAAGCTCCAGCCCCGAGGACAGCACTACGCCAACATCTGGCAGGGAG
AGTTTCCTGTCACCAACACTGCTGAGGACGGATTCCAGGGCACCGCTCCTGTGGATG
CTTTTCCCCCTAACGGTTACGGACTGTACAACATTGTTGGAAACGCCTGGGAGTGGA
CCTCGGACTGGTGGACTGTGTACCATTCCGTTGAGGAGACCCTCAACCCCAAGGGTC
CCCCTTCTGGAAAGGATCGAGTGAAGAAGGGAGGCTCGTACATGTGCCACAAGTCC
TACTGTTACCGATACCGATGCGCCGCTCGATCTCAGAACACCCCCGACTCCTCTGCC
TCGAACCTCGGATTCCGATGTGCTGCTGACCGACTGCCCACTCACCACCACCACCAC
CACCACGACGAGCTGTAA
SEQ ID NO 55: Monodelphis domestica Lip2-MdFGE-His6-HDEL fusion construct
MKLSTILFTACATLAAAAARGLGSEAGSAAADAAHPAGTCGCGSPQRPGTAAHRYSRE
ANVAEPASAERPVLTSQMAHIPAGVFTMGTDEPQIKQDGEGPARRVRINSFYMDLYEVS
NAEFERFVNSTGYVTEAEKFGDSFVFDSMLSDQVKSDIHQAVAAAPWWLPVKGANWR
HPEGPDSSILHRRDHPVLHVSWNDAVAYCTWAGKRLPTEAEWEYSCRGGLENRLFPW
GNKLQPKGQHYANIWQGEFPVSNTGEDGYQGTAPVTAFPPNGYGLYNIVGNAWEWTS
DWWTVHHSADETLDPKGPPSGSDRVKKGGSYMCHKSYCYRYRCAARSQNTPDSSASN
LGFRCAADRLPDTHHHHHHHDEL
SEQ ID NO 56: Coding sequence for the Lip2-MdFGE-His6-HDEL fusion protein
ATGAAGCTTTCCACCATCCTCTTCACAGCCTGCGCTACCCTGGCTGCCGCCGCCGCC
CGAGGTCTGGGTTCCGAGGCCGGTTCCGCCGCCGCCGACGCCGCTCACCCTGCTGGC
ACTTGTGGTTGTGGTTCCCCTCAGCGACCCGGCACCGCCGCTCACCGATACTCTCGA
GAGGCTAACGTGGCTGAGCCTGCTTCTGCCGAGCGACCTGTGCTGACTTCGCAGATG
GCTCACATCCCCGCCGGTGTCTTCACCATGGGAACTGACGAGCCCCAGATCAAGCA
GGATGGAGAGGGACCTGCCCGACGAGTTCGAATTAACTCGTTTTACATGGACCTCTA
CGAGGTCTCCAACGCTGAGTTCGAGCGATTTGTTAACTCCACCGGTTACGTCACTGA
GGCCGAGAAGTTCGGAGACTCTTTCGTTTTTGATTCCATGCTGTCTGACCAGGTGAA
GTCCGATATCCATCAGGCTGTGGCCGCTGCCCCCTGGTGGCTCCCTGTCAAGGGAGC
TAACTGGCGACACCCTGAGGGACCTGACTCCTCTATTCTGCACCGACGAGATCATCC
CGTCCTCCACGTGTCTTGGAACGACGCTGTGGCCTACTGTACCTGGGCTGGAAAGCG
ACTGCCTACTGAGGCTGAGTGGGAGTACTCCTGCCGAGGCGGTCTGGAGAACCGAC
TCTTTCCCTGGGGCAACAAGCTCCAGCCTAAGGGTCAGCACTACGCTAACATCTGGC
AGGGCGAGTTCCCCGTCTCCAACACCGGAGAGGACGGCTACCAGGGCACCGCTCCT
GTGACTGCCTTTCCCCCTAACGGCTACGGTCTGTACAACATTGTGGGTAACGCTTGG
GAGTGGACCTCCGACTGGTGGACTGTTCACCATTCTGCCGACGAGACCCTCGATCCC
AAGGGACCCCCTTCTGGCTCGGATCGAGTTAAGAAGGGAGGCTCGTACATGTGCCA
CAAGTCCTACTGTTACCGATACCGATGCGCTGCCCGATCTCAGAACACCCCTGACTC
TTCCGCCTCTAACCTGGGCTTCCGATGTGCTGCTGACCGACTGCCTGACACTCATCA
CCATCATCACCACCACGACGAGCTGTAA
SEQ ID NO 57: Gallus gallus Lip2-GgFGE-His6-HDEL fusion construct
MKLSTILFTACATLAAAGKETAPGGNCGCSASRSRGGEREAVATVRRYSAAANDGRSS
GRGPMVAIPGGVFTMGTDEPEIQQDGEWPARRVHVNSFYMDQYEVSNQEFERFVNSTG
YLTEAEKFGDSFVFEGMLSEEVKAEIHQAVAAAPWWLPVKGANWRQPEGPGSSILSRM
DHPVLHVSWNDAVAFCTWAGKRLPTEAEWEYGCRGGLEKRLFPWGNKLQPKGQHYA
NIWQGVFPTNNTAEDGYKGTAPVTAFPPNGYGLYNIVGNAWEWTSDWWAVHHSADE
AHNPKGPSSGTDRVKKGGSYMCHKSYCYRYRCAARSQNTPDSSASNLGFRCAADALPD
PQHHHHHHHDEL
SEQ ID NO 58: Coding sequence for the Lip2-GgFGE-His6-HDEL fusion protein
ATGAAGCTTTCCACCATCCTCTTCACAGCCTGCGCTACCCTGGCTGCCGCCGGCAAG
GAGACTGCCCCTGGCGGTAACTGCGGTTGTTCTGCTTCCCGATCCCGAGGTGGAGAG
CGAGAGGCCGTTGCTACTGTCCGACGATACTCCGCCGCTGCCAACGACGGCCGATCC
TCTGGCCGAGGTCCCATGGTGGCTATCCCTGGCGGTGTTTTCACCATGGGAACTGAC
GAGCCCGAGATTCAGCAGGATGGCGAGTGGCCTGCTCGACGAGTCCACGTGAACTC
GTTTTACATGGACCAGTACGAGGTTTCTAACCAGGAGTTCGAGCGATTTGTCAACTC
TACCGGATACCTGACTGAGGCCGAGAAGTTCGGCGACTCTTTCGTTTTTGAGGGAAT
GCTCTCGGAGGAAGTCAAGGCCGAGATCCATCAGGCTGTTGCTGCCGCTCCTTGGTG
GCTGCCTGTGAAGGGTGCTAACTGGCGACAGCCTGAGGGACCTGGCTCGTCCATTCT
GTCCCGAATGGACCACCCCGTTCTCCATGTCTCTTGGAACGATGCCGTCGCTTTCTGT
ACCTGGGCTGGCAAGCGACTGCCTACTGAGGCTGAGTGGGAGTACGGATGCCGAGG
CGGCCTGGAGAAGCGACTCTTTCCCTGGGGCAACAAGCTCCAGCCTAAGGGTCAGC
ACTACGCCAACATCTGGCAGGGCGTCTTCCCCACCAACAACACTGCTGAGGACGGC
TACAAGGGCACCGCCCCTGTGACTGCTTTTCCCCCTAACGGTTACGGACTGTACAAC
ATTGTGGGTAACGCCTGGGAGTGGACCTCTGACTGGTGGGCTGTTCACCATTCTGCC
GATGAGGCTCACAACCCCAAGGGACCTTCTTCGGGCACCGACCGAGTGAAGAAGGG
TGGATCGTACATGTGCCATAAGTCCTACTGTTACCGATACCGATGCGCCGCTCGATC
CCAGAACACCCCCGATTCCTCTGCCTCTAACCTCGGTTTCCGATGTGCCGCCGACGC
CCTCCCCGACCCTCAGCATCACCATCACCATCATCACGACGAGCTGTAG
SEQ ID NO 59: Dendroctonus ponderosa Lip2-DpFGE-His6-HDEL fusion construct
MKLSTILFTACATLAAAICDCGCSLNRDGQCNSEDNEINPSQKYKRDLNENPADNFDKS
QMALIGKGIFEMGTNKPVFPSDFEGPARNVTIENSFYLDLYEVSNQQFYDFVRTTNYKTE
AEQFGDSFVFEMSLPENQRNEHQDIRAAQAPWWIKLPDAYWKHPEGPKSTIEDRIVINHP
VAHVSWNDAVAYCEYVGKRLPTEAEWEMACRGGLRQKMYPWGNKLQPKGQHWANI
WQGEFPKENTAEDGYIFTCPVDKFPPNQFGLYNMAGNVWEWVQDDWQTDPQNSRVK
KGGSFLCHQSYCWRYRCAARSFNTKDSSAANLGFRCAADARBEHHHHHDEL
SEQ ID NO 60: Coding sequence for the Lip2-DpFGE-His6-HDEL fusion protein
ATGAAGCTTTCCACCATCCTCTTCACAGCCTGCGCTACCCTGGCTGCCGCCATTTGCG
ACTGCGGCTGCTCCCTGAACCGAGACGGCCAGTGTAACTCCGAGGACAACGAGATT
AACCCCTCCCAGAAGTACAAGCGAGACCTGAACGAGAACCCCGCCGACAACTTCGA
TAAGTCTCAGATGGCTCTCATCGGCAAGGGAATTTTTGAGATGGGCACCAACAAGCC
CGTTTTCCCTTCGGACTTTGAGGGTCCTGCCCGAAACGTCACTATCGAGAACTCCTTC
TACCTGGACCTCTACGAGGTCTCTAACCAGCAGTTCTACGATTTTGTGCGAACCACT
AACTACAAGACCGAGGCTGAGCAGTTCGGTGACTCGTTCGTCTTTGAGATGTCCCTG
CCCGAGAACCAGCGAAACGAGCACCAGGACATCCGAGCTGCTCAGGCTCCTTGGTG
GATTAAGCTCCCTGATGCTTACTGGAAGCATCCCGAGGGACCTAAGTCGACCATTGA
GGACCGAATGAACCACCCCGTCGCCCATGTGTCCTGGAACGATGCCGTGGCTTACTG
TGAGTACGTTGGCAAGCGACTGCCTACTGAGGCTGAGTGGGAGATGGCTTGCCGAG
GCGGTCTGCGACAGAAGATGTACCCCTGGGGAAACAAGCTCCAGCCTAAGGGCCAG
CACTGGGCCAACATCTGGCAGGGAGAGTTCCCCAAGGAGAACACCGCTGAGGACGG
ATACATTTTTACTTGTCCTGTGGATAAGTTCCCTCCCAACCAGTTTGGCCTCTACAAC
ATGGCCGGTAACGTTTGGGAGTGGGTCCAGGACGATTGGCAGACCGACCCCCAGAA
CTCCCGAGTTAAGAAGGGAGGCTCTTTCCTGTGCCATCAGTCGTACTGTTGGCGATA
CCGATGCGCCGCTCGATCTTTCAACACCAAGGACTCCTCTGCCGCTAACCTCGGATT
CCGATGTGCTGCTGACGCCCGACACCACCACCACCACCACCACGACGAGCTGTAG
SEQ ID NO 61: Columba livia Lip2-ClFGE-His6-HDEL fusion construct
MKLSTILFTACATLAAAMVVIPGGVFTMGTDEPAIQQDGEWPVRKVHVNSFYMDRYEV
SNEDFERFVNSTGYVTEAEKFGDSFVFEGMLSEEVKAEIHQAVAAAPWWLPVKGANW
KHPEGPDSNISNRMDHPVLHVSWNDAVAFCTWAGKRLPTEAEWEYSCRGGLENRLFP
WGNKLQPKGQHYANIWQGVFPTNNTAEDGYKGTAPVTAFPPNGYGLYNIVGNAWEW
TADWWAVHHSTEEVHNPKGPSSGTDRVKKGGSYMCHKSYCYRYRCAARSQNTPDSSA
SNLGFRCAADASPELPHHHHHHHDEL
SEQ ID NO 62: Coding sequence for the Lip2-ClFGE-His6-HDEL fusion protein
ATGAAGCTTTCCACCATCCTCTTCACAGCCTGCGCTACCCTGGCTGCCGCCATGGTC
GTTATTCCCGGAGGAGTTTTTACTATGGGTACTGATGAGCCCGCTATCCAGCAGGAC
GGAGAGTGGCCCGTGCGAAAGGTTCACGTTAACTCTTTCTACATGGACCGATACGAG
GTCTCGAACGAGGATTTCGAGCGATTTGTTAACTCCACCGGCTACGTCACTGAGGCT
GAGAAGTTTGGTGACTCGTTCGTCTTTGAGGGAATGCTGTCCGAGGAAGTCAAGGCT
GAGATCCACCAGGCTGTGGCCGCTGCCCCCTGGTGGCTCCCTGTGAAGGGAGCTAA
CTGGAAGCATCCCGAGGGCCCTGACTCTAACATTTCGAACCGAATGGATCACCCCGT
CCTGCATGTGTCCTGGAACGATGCTGTTGCCTTCTGTACCTGGGCTGGCAAGCGACT
GCCTACTGAGGCCGAGTGGGAGTACTCTTGCCGAGGCGGTCTGGAGAACCGACTCTT
TCCCTGGGGCAACAAGCTGCAGCCTAAGGGTCAGCACTACGCTAACATCTGGCAGG
GTGTGTTCCCCACCAACAACACTGCCGAGGACGGCTACAAGGGCACCGCTCCTGTG
ACTGCCTTTCCCCCTAACGGTTACGGACTCTACAACATTGTTGGAAACGCTTGGGAG
TGGACCGCTGACTGGTGGGCTGTGCACCATTCTACTGAGGAAGTCCACAACCCCAA
GGGACCTTCCTCTGGCACCGATCGAGTCAAGAAGGGAGGCTCCTACATGTGCCATA
AGTCTTACTGTTACCGATACCGATGCGCTGCCCGATCCCAGAACACCCCCGACTCGT
CCGCCTCTAACCTGGGATTCCGATGTGCTGCCGACGCTTCGCCTGAGCTGCCCCACC
ACCACCATCACCATCACGACGAGCTGTAA
SEQ ID NO 63: MNS1-ClFGE fusion construct
MSFNIPKTTPNFSAKARKLEDQLWQASGLEKSKDSTLPLYKDKPYGEGFVARTTSGRRR
RNIIYGVVVGLLFWAIYTFSRSLDGNVSLKDGIKDYEFKGWKGRGKPKTNWVAEQNAV
KQAFVDSWNGYHKYAWGKDVYKPQTKTGKNIVIGPKPLGWFIVDSLDSMVVIPGGVFT
MGTDEPAIQQDGEWPVRKVHVNSFYMDRYEVSNEDFERFVNSTGYVTEAEKFGDSFVF
EGMLSEEVKAEIHQAVAAAPWWLPVKGANWKHPEGPDSNISNRMDHPVLHVSWNDA
VAFCTWAGKRLPTEAEWEYSCRGGLENRLFPWGNKLQPKGQHYANIWQGVFPTNNTA
EDGYKGTAPVTAFPPNGYGLYNIVGNAWEWTADWWAVHHSTEEVHNPKGPSSGTDRV
KKGGSYMCHKSYCYRYRCAARSQNTPDSSASNLGFRCAADASPELP
SEQ ID NO 64: Coding sequence for the MNS1-ClFGE fusion protein
ATGTCGTTCAACATTCCCAAGACCACCCCCAACTTCTCGGCTAAGGCTCGAAAGCTG
GAGGATCAGCTCTGGCAGGCTTCTGGACTCGAGAAGTCCAAGGACTCTACCCTGCCT
CTCTACAAGGATAAGCCCTACGGAGAGGGCTTCGTGGCTCGAACCACTTCCGGCCG
ACGACGACGAAACATCATCTACGGCGTCGTGGTTGGTCTGCTCTTCTGGGCCATCTA
CACCTTTTCTCGATCGCTGGACGGTAACGTCTCTCTCAAGGACGGAATTAAGGATTA
CGAGTTCAAGGGCTGGAAGGGTCGAGGAAAGCCCAAGACTAACTGGGTGGCCGAGC
AGAACGCTGTTAAGCAGGCCTTTGTCGACTCCTGGAACGGCTACCATAAGTACGCCT
GGGGCAAGGATGTGTACAAGCCCCAGACCAAGACTGGAAAGAACATGGGCCCCAA
GCCTCTGGGATGGTTCATCGTGGACTCTCTGGATTCCATGGTCGTTATTCCCGGAGG
AGTTTTTACTATGGGTACTGATGAGCCCGCTATCCAGCAGGACGGAGAGTGGCCCGT
GCGAAAGGTTCACGTTAACTCTTTCTACATGGACCGATACGAGGTCTCGAACGAGGA
TTTCGAGCGATTTGTTAACTCCACCGGCTACGTCACTGAGGCTGAGAAGTTTGGTGA
CTCGTTCGTCTTTGAGGGAATGCTGTCCGAGGAAGTCAAGGCTGAGATCCACCAGGC
TGTGGCCGCTGCCCCCTGGTGGCTCCCTGTGAAGGGAGCTAACTGGAAGCATCCCGA
GGGCCCTGACTCTAACATTTCGAACCGAATGGATCACCCCGTCCTGCATGTGTCCTG
GAACGATGCTGTTGCCTTCTGTACCTGGGCTGGCAAGCGACTGCCTACTGAGGCCGA
GTGGGAGTACTCTTGCCGAGGCGGTCTGGAGAACCGACTCTTTCCCTGGGGCAACAA
GCTGCAGCCTAAGGGTCAGCACTACGCTAACATCTGGCAGGGTGTGTTCCCCACCAA
CAACACTGCCGAGGACGGCTACAAGGGCACCGCTCCTGTGACTGCCTTTCCCCCTAA
CGGTTACGGACTCTACAACATTGTTGGAAACGCTTGGGAGTGGACCGCTGACTGGTG
GGCTGTGCACCATTCTACTGAGGAAGTCCACAACCCCAAGGGACCTTCCTCTGGCAC
CGATCGAGTCAAGAAGGGAGGCTCCTACATGTGCCATAAGTCTTACTGTTACCGATA
CCGATGCGCTGCCCGATCCCAGAACACCCCCGACTCGTCCGCCTCTAACCTGGGATT
CCGATGTGCTGCCGACGCTTCGCCTGAGCTGCCC
SEQ ID NO 65: c-myc protein tag
EQKLISEEDL
SEQ ID NO 66: Coding sequence for the c-myc protein tag
GAACAAAAACTCATCTCAGAAGAGGATCTGTAA
SEQ ID NO 67: MNS1-ClFGE-c-myc fusion construct
MSFNIPKTTPNFSAKARKLEDQLWQASGLEKSKDSTLPLYKDKPYGEGFVARTTSGRRR
RNIIYGVVVGLLFWAIYTFSRSLDGNVSLKDGIKDYEFKGWKGRGKPKTNWVAEQNAV
KQAFVDSWNGYHKYAWGKDVYKPQTKTGKNMGPKPLGWFIVDSLDSMVVIPGGVFT
MGTDEPAIQQDGEWPVRKVHVNSFYMDRYEVSNEDFERFVNSTGYVTEAEKFGDSFVF
EGMLSEEVKAEIHQAVAAAPWWLPVKGANWKHPEGPDSNISNRMDHPVLHVSWNDA
VAFCTWAGKRLPTEAEWEYSCRGGLENRLFPWGNKLQPKGQHYANIWQGVFPTNNTA
EDGYKGTAPVTAFPPNGYGLYNIVGNAWEWTADWWAVHHSTEEVHNPKGPSSGTDRV
KKGGSYMCHKSYCYRYRCAARSQNTPDSSASNLGFRCAADASPELPEQKLISEEDL
SEQ ID NO 68: Coding sequence for the MNS1-ClFGE-c-myc fusion protein
ATGTCGTTCAACATTCCCAAGACCACCCCCAACTTCTCGGCTAAGGCTCGAAAGCTG
GAGGATCAGCTCTGGCAGGCTTCTGGACTCGAGAAGTCCAAGGACTCTACCCTGCCT
CTCTACAAGGATAAGCCCTACGGAGAGGGCTTCGTGGCTCGAACCACTTCCGGCCG
ACGACGACGAAACATCATCTACGGCGTCGTGGTTGGTCTGCTCTTCTGGGCCATCTA
CACCTTTTCTCGATCGCTGGACGGTAACGTCTCTCTCAAGGACGGAATTAAGGATTA
CGAGTTCAAGGGCTGGAAGGGTCGAGGAAAGCCCAAGACTAACTGGGTGGCCGAGC
AGAACGCTGTTAAGCAGGCCTTTGTCGACTCCTGGAACGGCTACCATAAGTACGCCT
GGGGCAAGGATGTGTACAAGCCCCAGACCAAGACTGGAAAGAACATGGGCCCCAA
GCCTCTGGGATGGTTCATCGTGGACTCTCTGGATTCCATGGTCGTTATTCCCGGAGG
AGTTTTTACTATGGGTACTGATGAGCCCGCTATCCAGCAGGACGGAGAGTGGCCCGT
GCGAAAGGTTCACGTTAACTCTTTCTACATGGACCGATACGAGGTCTCGAACGAGGA
TTTCGAGCGATTTGTTAACTCCACCGGCTACGTCACTGAGGCTGAGAAGTTTGGTGA
CTCGTTCGTCTTTGAGGGAATGCTGTCCGAGGAAGTCAAGGCTGAGATCCACCAGGC
TGTGGCCGCTGCCCCCTGGTGGCTCCCTGTGAAGGGAGCTAACTGGAAGCATCCCGA
GGGCCCTGACTCTAACATTTCGAACCGAATGGATCACCCCGTCCTGCATGTGTCCTG
GAACGATGCTGTTGCCTTCTGTACCTGGGCTGGCAAGCGACTGCCTACTGAGGCCGA
GTGGGAGTACTCTTGCCGAGGCGGTCTGGAGAACCGACTCTTTCCCTGGGGCAACAA
GCTGCAGCCTAAGGGTCAGCACTACGCTAACATCTGGCAGGGTGTGTTCCCCACCAA
CAACACTGCCGAGGACGGCTACAAGGGCACCGCTCCTGTGACTGCCTTTCCCCCTAA
CGGTTACGGACTCTACAACATTGTTGGAAACGCTTGGGAGTGGACCGCTGACTGGTG
GGCTGTGCACCATTCTACTGAGGAAGTCCACAACCCCAAGGGACCTTCCTCTGGCAC
CGATCGAGTCAAGAAGGGAGGCTCCTACATGTGCCATAAGTCTTACTGTTACCGATA
CCGATGCGCTGCCCGATCCCAGAACACCCCCGACTCGTCCGCCTCTAACCTGGGATT
CCGATGTGCTGCCGACGCTTCGCCTGAGCTGCCCGAACAAAAACTCATCTCAGAAG
AGGATCTGTAA

1. A method for making a type I sulfatase, or a functional fragment thereof, in an active form, the method comprising:
a) providing a fungal cell genetically engineered such that, when transformed with a polynucleotide encoding a type I sulfatase, or a functional fragment thereof, the cell has the ability to produce the type I sulfatase, or a functional fragment thereof, in an active form, or an increased level of the type I sulfatase, or a functional fragment thereof, in an active form; and
b) introducing into the cell a nucleic acid encoding the type I sulfatase, or a functional fragment thereof, wherein the encoded type I sulfatase, or the functional fragment thereof, without an activation step, is an inactive form,
wherein, after the introduction, the cell produces, or produces at an increased level, the type I sulfatase, or functional fragment thereof, in an active form.

2. A method for making a type I sulfatase, or a functional fragment thereof, in an active form, the method comprising:
a) providing a fungal cell genetically engineered to produce a protein with the type I sulfatase activating activity of a Formylglycine Generating Enzyme (FGE); and
b) introducing into the cell a nucleic acid encoding a type I sulfatase, or a functional fragment thereof, wherein the encoded type I sulfatase, or the functional fragment thereof, without an activation step, is in an inactive form, or
c) providing a fungal cell genetically engineered to produce a type I sulfatase, or a functional fragment thereof, wherein the type I sulfatase or functional fragment thereof, without an activation step, is in an inactive form; and
d) introducing into the cell a nucleic acid encoding a protein with the type I sulfatase activating activity of a Formylglycine Generating Enzyme (FGE),
wherein, after the introduction, the cell produces, or produces at an increased level, the type I sulfatase, or the functional fragment thereof, in an active form.

3. (canceled)

4. The method of claim 2, wherein the protein with the type I sulfatase activating activity of a FGE comprises:
(a) a mature wild type FGE polypeptide;
(b) a functional fragment of a mature wild type FGE polypeptide comprising at least 50 consecutive amino acids of the mature wild type FGE;
(c) a polypeptide with at least 80% identity to the mature wild type FGE polypeptide of (a);
(d) a polypeptide with at least 90% identity to the functional fragment of (b);
(e) the mature wild type FGE polypeptide of (a) but with no more than 10 conservative substitutions; or
(f) the functional fragment of (b) but with no more than 5 conservative substitutions.

5. The method of claim 4, wherein the mature wild type FGE polypeptide is:
(i) mature wild type protein SCO7548;
(ii) mature wild type protein Rv0712;
(iii) mature wild type sulfatase modifying factor 1;
(iv) mature wild type C-alpha-formylglycine-generating enzyme; or
(v) mature wild type sulfatase-modifying factor 1.

6.-9. (canceled)

10. The method of claim 2, wherein the protein with the type I sulfatase activating activity of a FGE is:
(i) a prokaryotic protein with the type I sulfatase activating activity of a FGE;
(ii) a prokaryotic protein with the type I sulfatase activating activity of a FGE, the prokaryote being Mycobacterium tuberculosis or Streptomyces coelicolor;
(iii) a protein with the type I sulfatase activating activity of a eukaryotic FGE; or
(iv) a protein with the type I sulfatase activating activity of a eukaryotic FGE, the eukaryote being Homo sapiens, Bos taurus, Hemicentrotus pulcherrimus, Tupaia chinensis, Monodelphis domestica, Gallus gallus, Dendroctonus ponderosa, or Columba livia.

11.-13. (canceled)

14. The method of claim 2, wherein the protein with the type I sulfatase activating activity of a FGE further comprises an ER targeting motif.

15. The method of claim 14, wherein the ER targeting motif:
(i) is fused to the C-terminus of the protein with the type I sulfatase activating activity of a FGE polypeptide;
(ii) is fused to the N-terminus of the protein with the type I sulfatase activating activity of a FGE polypeptide;
(iii) comprises HDEL (SEQ ID NO: 1);
(iv) comprises KDEL (SEQ ID NO: 3);
(v) comprises DDEL (SEQ ID NO: 4) or RDEL (SEQ ID NO: 33);
(vi) comprises a yeast MNS1 transmembrane anchor polypeptide;
(vii) comprises a yeast MNS1 transmembrane anchor polypeptide comprising the Yarrowia lipolytica MNS1 transmembrane anchor polypeptide;
(viii) comprises a yeast WBP1 transmembrane anchor polypeptide; or
(ix) comprises a yeast WBP1 transmembrane anchor polypeptide comprising the Yarrowia lipolytica WBP1 transmembrane anchor polypeptide.

16.-27. (canceled)

28. The method of claim 1,
(i) the method further comprising introducing into the cell a nucleic acid encoding a polypeptide capable of effecting mannosyl phosphorylation, or a functional fragment thereof;
(ii) the method further comprising introducing into the cell a nucleic acid encoding a polypeptide capable of effecting mannosyl phosphorylation, or a functional fragment thereof, wherein the polypeptide capable of effecting mannosyl phosphorylation is selected from the group consisting of MNN4, PNO1, and MNN6;
(iii) the method further comprising introducing into the cell a nucleic acid encoding a mannosidase, or a functional fragment thereof, capable of hydrolyzing a terminal mannose-1-phospho-6-mannose moiety to a terminal phospho-6-mannose;
(iv) the method further comprising introducing into the cell a nucleic acid encoding a mannosidase, or a functional fragment thereof, capable of hydrolyzing a terminal mannose-1-phospho-6-mannose moiety to a terminal phospho-6-mannose, wherein the mannosidase is the family 92 glycoside hydrolase CcMan5 from Cellulosimicrobium cellulans;
(v) the method further comprising introducing into the cell a nucleic acid encoding a mannosidase, or a functional fragment thereof, capable of hydrolyzing a terminal mannose-1-phospho-6-mannose moiety to a terminal phospho-6-mannose, wherein the mannosidase is also capable of removing a mannose residue bound by an alpha 1,2 linkage to the underlying mannose in the terminal mannose-1-phospho-6-mannose moiety;
(vi) the method further comprising introducing into the cell a nucleic acid encoding a mannosidase, or a functional fragment thereof, capable of hydrolyzing a terminal mannose-1-phospho-6-mannose moiety to a terminal phospho-6-mannose, wherein the mannosidase is also capable of removing a mannose residue bound by an alpha 1,2 linkage to the underlying mannose in the terminal mannose-1-phospho-6-mannose moiety, wherein the mannosidase is a family 38 glycoside hydrolase selected from the group consisting of a Canavalia ensiformis (Jack Bean) mannosidase and Yarrowia lipolytica AMS1 mannosidase;
(vii) the method further comprising introducing into the cell a nucleic acid encoding a mannosidase, or a functional fragment thereof, capable of hydrolyzing a terminal mannose-1-phospho-6-mannose moiety to a terminal phospho-6-mannose, and further comprising introducing into the cell a nucleic acid encoding a second mannosidase, or a functional fragment thereof, that is capable of removing a mannose residue bound by an alpha 1,2 linkage to the underlying mannose in the terminal mannose-1-phospho-6-mannose moiety;
(viii) the method further comprising introducing into the cell a nucleic acid encoding a mannosidase, or a functional fragment thereof, capable of hydrolyzing a terminal mannose-1-phospho-6-mannose moiety to a terminal phospho-6-mannose, and further comprising introducing into the cell a nucleic acid encoding a second mannosidase, or a functional fragment thereof, that is capable of removing a mannose residue bound by an alpha 1,2 linkage to the underlying mannose in the terminal mannose-1-phospho-6-mannose moiety, wherein the second mannosidase is selected from the group consisting of the family 38 glycoside hydrolase Canavalia ensiformis (Jack Bean) mannosidase, the family 38 glycoside hydrolase Yarrowia lipolytica AMS1 mannosidase, the family 47 glycoside hydrolase Aspergillus satoi As mannosidase, and the family 92 glycoside hydrolase Cellulosimicrobium cellulans CcMan4 mannosidase; or
(ix) wherein the cell comprises a deficiency in OCH1 activity.

29.-39. (canceled)

40. The method of claim 1, wherein the fungal cell is:
(i) a yeast cell;
(ii) a yeast cell that is a Yarrowia lipolytica cell;
(iii) a yeast cell of a methylotrophic yeast;
(iv) a yeast cell of a methylotrophic yeast selected from the group comprising Pichia pastoris, Pichia methanolica, Ogataea minuta, and Hansenula polymorpha;
(v) a cell of a filamentous fungus; or
(vi) a cell of a filamentous fungus selected from a group consisting of: Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowii, Aspergillus tamari, Aspergillus terreus, Aspergillus ustus, Aspergillus versicolor, Trichoderma, and Neurospora.

41.-48. (canceled)

49. The method of claim 1, wherein,
(i) after step (b), the cell, or the progeny thereof, is cultivated at a high pO₂; or
(ii) after step (b), the cell, or the progeny thereof, is cultivated at a high pO₂ that is 5%-40%.

50.-54. (canceled)

55. The method of claim 4, wherein the protein with the type I sulfatase activity of a FGE:
(i) comprises any one of (a)-(f) and the mature wild type FGE polypeptide is a mature wild type Columba livia FGE polypeptide; and
(ii) further comprises a yeast MNS1 transmembrane anchor polypeptide.

56. The method of claim 55, wherein the protein with the type I sulfatase activating activity of a FGE comprises the amino acid sequence set forth in SEQ ID NO: 63.

57. An active type I sulfatase, or a functional fragment thereof, produced by the method of claim 1.

58. A method of treating a subject having, or suspected of having, a disorder treatable with a type I sulfatase, the method comprising administering to the subject the active type I sulfatase, or functional fragment thereof, of claim 57 to the subject.

59. The method of claim 58, wherein
(i) the disorder is a lysosomal storage disorder;
(ii) the disorder is selected from the group consisting of metachromatic leukodystrophy, Hunter disease, Sanfilippo disease A & D, Morquio disease A, Maroteaux-Lamy disease, X-linked ichthyosis, Chondrodysplasia Punctata 1, or Multiple Sulfatase Deficiency; or
(iii) the subject is a human.

60.-61. (canceled)

62. An isolated fungal cell comprising a nucleic acid encoding a protein with the type I sulfatase activating activity of a FGE.

63. The fungal cell of claim 62, wherein the protein with the type I sulfatase activating activity of a FGE comprises:
(a) a mature wild type FGE polypeptide;
(b) a functional fragment of a mature wild type FGE polypeptide comprising at least 50 consecutive amino acids of the mature wild type FGE;
(c) a polypeptide with at least 70% identity to (a);
(d) a polypeptide with at least 85% identity to (b);
(e) (a) but with no more than 10 conservative substitutions; or
(f) (b) but with no more than 5 conservative substitutions.

64. The fungal cell of claim 62, the fungal cell further comprising a nucleic acid encoding a type I sulfatase, or a functional fragment thereof, wherein the encoded type I sulfatase, or functional fragment thereof, without the action of an activating factor on it, is in an inactive form.

65. The fungal cell of claim 63, wherein the mature wild type FGE polypeptide is:
(i) mature wild type protein SCO7548;
(ii) mature wild type protein Rv0712;
(iii) mature wild type sulfatase modifying factor 1;
(iv) mature wild type C-alpha-formylglycine-generating enzyme; or
(v) mature wild type sulfatase-modifying factor 1.

66.-69. (canceled)

70. The fungal cell of claim 62, wherein the protein with the type I sulfatase activating activity of a FGE is:
(i) a prokaryotic protein with the type I sulfatase activating activity of a FGE;
(ii) a prokaryotic protein with the type I sulfatase activating activity of a FGE, the prokaryote being Mycobacterium tuberculosis or Streptomyces coelicolor;
(iii) a protein with the type I sulfatase activating activity of a eukaryotic FGE; or
(iv) a protein with the type I sulfatase activating activity of a eukaryotic FGE, the eukaryote being Homo sapiens, Bos taurus, Hemicentrotus pulcherrimus, Tupaia chinensis, Monodelphis domestica, Gallus gallus, Dendroctonus ponderosa, or Columba livia.

71.-73. (canceled)

74. The fungal cell of claim 62, wherein the protein with the type I sulfatase activating activity of a FGE further comprises an ER targeting motif.

75. The fungal cell of claim 74, wherein the ER targeting motif:
(i) is fused to the C-terminus of the protein with the type I sulfatase activating activity of a FGE polypeptide;
(ii) is fused to the N-terminus of the protein with the type I sulfatase activating activity of a FGE polypeptide;
(iii) comprises HDEL (SEQ ID NO: 1);
(iv) comprises KDEL (SEQ ID NO: 3);
(v) comprises DDEL (SEQ ID NO: 4) or RDEL (SEQ ID NO: 33);
(vi) comprises a yeast MNS1 transmembrane anchor polypeptide;
(vii) comprises a yeast MNS1 transmembrane anchor polypeptide comprising the Yarrowia lipolytica MNS1 transmembrane anchor polypeptide;
(viii) comprises a yeast WBP1 transmembrane anchor polypeptide; or
(ix) comprises a yeast WBP1 transmembrane anchor polypeptide comprising the Yarrowia lipolytica WBP1 transmembrane anchor polypeptide.

76.-87. (canceled)

88. The fungal cell of claim 62,
(i) the fungal cell further comprising a nucleic acid encoding a polypeptide capable of effecting mannosyl phosphorylation, or a functional fragment thereof;
(ii) the fungal cell further comprising a nucleic acid encoding a polypeptide capable of effecting mannosyl phosphorylation, or a functional fragment thereof, wherein the polypeptide capable of effecting mannosyl phosphorylation is selected from the group consisting of MNN4, PNO1, and MNN6;
(iii) the fungal cell further comprising a nucleic acid encoding a mannosidase, or a functional fragment thereof, capable of hydrolyzing a terminal mannose-1-phospho-6-mannose moiety to a terminal phospho-6-mannose;
(iv) the fungal cell further comprising a nucleic acid encoding a mannosidase, or a functional fragment thereof, capable of hydrolyzing a terminal mannose-1-phospho-6-mannose moiety to a terminal phospho-6-mannose, wherein the mannosidase is the family 92 glycoside hydrolase CcMan5 from Cellulosimicrobium cellulans;
(v) the fungal cell further comprising a nucleic acid encoding a mannosidase, or a functional fragment thereof, capable of hydrolyzing a terminal mannose-1-phospho-6-mannose moiety to a terminal phospho-6-mannose, wherein the mannosidase is also capable of removing a mannose residue bound by an alpha 1,2 linkage to the underlying mannose in the terminal mannose-1-phospho-6-mannose moiety;
(vi) the fungal cell further comprising a nucleic acid encoding a mannosidase, or a functional fragment thereof, capable of hydrolyzing a terminal mannose-1-phospho-6-mannose moiety to a terminal phospho-6-mannose, wherein the mannosidase is also capable of removing a mannose residue bound by an alpha 1,2 linkage to the underlying mannose in the terminal mannose-1-phospho-6-mannose moiety, wherein the mannosidase is a family 38 glycoside hydrolase selected from the group consisting of a Canavalia ensiformis (Jack Bean) mannosidase and Yarrowia lipolytica AMS1 mannosidase;
(vii) the fungal cell further comprising a nucleic acid encoding a mannosidase, or a functional fragment thereof, capable of hydrolyzing a terminal mannose-1-phospho-6-mannose moiety to a terminal phospho-6-mannose, and further comprising a nucleic acid encoding a second mannosidase, or a functional fragment thereof, that is capable of removing a mannose residue bound by an alpha 1,2 linkage to the underlying mannose in the terminal mannose-1-phospho-6-mannose moiety;
(viii) the fungal cell further comprising a nucleic acid encoding a mannosidase, or a functional fragment thereof, capable of hydrolyzing a terminal mannose-1-phospho-6-mannose moiety to a terminal phospho-6-mannose, and further comprising a nucleic acid encoding a second mannosidase, or a functional fragment thereof, that is capable of removing a mannose residue bound by an alpha 1,2 linkage to the underlying mannose in the terminal mannose-1-phospho-6-mannose moiety, wherein the second mannosidase is selected from the group consisting of the family 38 glycoside hydrolase Canavalia ensiformis (Jack Bean) mannosidase, the family 38 glycoside hydrolase Yarrowia lipolytica AMS1 mannosidase, the family 47 glycoside hydrolase Aspergillus satoi As mannosidase, and the family 92 glycoside hydrolase Cellulosimicrobium cellulans CcMan4 mannosidase; or
(ix) wherein the cell comprises a deficiency in OCH1 activity.

89.-95. (canceled)

96. The fungal cell of claim 62, further comprising a nucleic acid encoding a trafficking protein, or a functional fragment thereof, wherein the trafficking protein or functional fragment thereof, directs the protein with the type I sulfatase activating activity of a FGE to the endoplasmic reticulum (ER) of the cell.

97. The fungal cell of claim 96, wherein:
(i) the trafficking protein is Protein Disulfide Isomerase (PDI);
(ii) the trafficking protein is Endoplasmic Reticulum Protein 44 (Erp44) or human SUMF2; or
(iii) the trafficking protein, or functional fragment thereof, binds to the protein with the type I sulfatase activating activity of a FGE

98.-99. (canceled)

100. The fungal cell of claim 62, wherein the fungal cell is:
(i) a yeast cell;
(ii) a yeast cell that is a Yarrowia lipolytica cell;
(iii) a yeast cell of a methylotrophic yeast;
(iv) a yeast cell of a methylotrophic yeast selected from the group comprising Pichia pastoris, Pichia methanolica, Ogataea minuta, and Hansenula polymorpha;
(v) a cell of a filamentous fungus; or
(vi) a cell of a filamentous fungus selected from a group consisting of: Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowii, Aspergillus tamari, Aspergillus terreus, Aspergillus ustus, Aspergillus versicolor, Trichoderma, and Neurospora.

101.-109. (canceled)

110. The fungal cell of claim 63, wherein the protein with the type I sulfatase activity of a FGE
(i) comprises any one of (a)-(f) and the mature wild type FGE polypeptide is a mature wild type Columba livia FGE polypeptide;
(ii) and further comprises a yeast MNS1 transmembrane anchor polypeptide.

111. The fungal cell of claim 110, wherein the protein with the type I sulfatase activating activity of a FGE comprises the amino acid sequence set forth in SEQ ID NO: 63.

112.-113. (canceled)

114. The method of claim 4, wherein the mature wild type FGE is
(i) a mature wild type FGE of Hemicentrotus pulcherrimus having the amino acid sequence set forth in SEQ ID NO: 13, a mature wild type FGE of Gallus gallus having the amino acid sequence set forth in SEQ ID NO: 47, a mature wild type FGE of Dendroctonus ponderosa having the amino acid sequence set forth in SEQ ID NO: 49, mature wild type FGE of Bos taurus having the amino acid sequence set forth in SEQ ID NO: 15 or a mature wild type FGE of Columba livia having the amino acid sequence set forth in SEQ ID NO: 51; or
(ii) a functional mature FGE having an amino acid sequence that is at least 80% identical to any one of the amino acid sequences of (i).

115. The method of claim 2, wherein the protein with the type I sulfatase activating activity of a FGE is encoded by a nucleotide sequence comprising
(i) the nucleic acid sequence set out in any one of SEQ ID NOs: 14, 48, 50 or 52; or
(ii) a nucleic acid sequence that is at least 80% identical to any one of the nucleic acid sequences of (i) and encodes a mature functional FGE; or
(iii) a nucleic acid sequence that hybridizes to a complement of any one of the nucleic acid sequences of (i) under high stringency and encodes a mature functional FGE.

116. The isolated fungal cell of claim 63, wherein the mature wild type FGE is
(i) a mature wild type FGE of Hemicentrotus pulcherrimus having the amino acid sequence set forth in SEQ ID NO: 13, a mature wild type FGE of Gallus gallus having the amino acid sequence set forth in SEQ ID NO: 47, a mature wild type FGE of Dendroctonus ponderosa having the amino acid sequence set forth in SEQ ID NO: 49, or a mature wild type FGE of Columba livia having the amino acid sequence set forth in SEQ ID NO: 51; or
(ii) a functional mature FGE having an amino acid sequence that is at least 80% identical to any one of the amino acid sequences of (i).

117. The isolated fungal cell of claim 62, wherein the protein with the type I sulfatase activating activity of a FGE is encoded by a nucleotide sequence comprising
(i) the nucleic acid sequence set out in any one of SEQ ID NOs: 14, 48, 50 or 52; or
(ii) a nucleic acid sequence that is at least 80% identical to any one of the nucleic acid sequences of (i) and encodes a mature functional FGE; or
(iii) a nucleic acid sequence that hybridizes to a complement of any one of the nucleic acid sequences of (i) under high stringency and encodes a mature functional FGE.
